SEQUENCE LISTING (71 sequences)

SEQ ID NO: 1 - protein, 107 aa, Artificial sequence
  variable domain of the light chain of the monoclonal antibody 4F11E12
DIQVTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 2 - protein, 118 aa, Artificial sequence [sequence omitted]

SEQ ID NO: 3 - protein, 253 aa, Toxoplasma gondii [sequence omitted]

SEQ ID NO: 4 - protein, 12 aa, Artificial sequence
  CDR antibody anti-SAG1
CRASQDISNYLN

SEQ ID NO: 5 - protein, 7 aa, Artificial sequence
  CDR antibody anti-SAG1
YTSRLHS

SEQ ID NO: 6 - protein, 9 aa, Artificial sequence
  CDR antibody anti-SAG1
QQGNTLPYT

SEQ ID NO: 7 - protein, 5 aa, Artificial sequence
  CDR antibody anti-SAG1
DYGMH

SEQ ID NO: 8 - protein, 16 aa, Artificial sequence
  CDR antibody anti-SAG1
IISTYSGDASYNQKFK

SEQ ID NO: 9 - protein, 9 aa, Artificial sequence
  CDR antibody anti-SAG1
SSTWYYFDY

SEQ ID NO: 10 - protein, 251 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSGGGGSGGGGSDIQVTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKGGGASHHHHHH

SEQ ID NO: 11 - protein, 241 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSDIQVTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKGGGASHHHHHH

SEQ ID NO: 12 - protein, 251 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSGGGGSGGGGSDIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKGGGASHHHHHH

SEQ ID NO: 13 - protein, 241 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSDIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKGGGASHHHHHH

SEQ ID NO: 14 - protein, 383 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSGGGGSGGGGSDIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKEPRGPTIKPCPPCKCGGGSSGGGSGGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGASHHHHHH

SEQ ID NO: 15 - protein, 373 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSDIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKEPRGPTIKPCPPCKCGGGSSGGGSGGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGASHHHHHH

SEQ ID NO: 16 - protein, 484 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSGGGGSGGGGSDIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGASHHHHHH

SEQ ID NO: 17 - protein, 251 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSGGGGSGGGGSDIQVTQTPSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKGGGASHHHHHH

SEQ ID NO: 18 - protein, 249 aa, Artificial sequence
  chimeric protein
QVQLQQSGAELVRPGTSVKISCKASGYTFTDYGMHWVKQRPGHGLEWIGIISTYSGDASYNQKFKGKATMTVDKSSSTAYMQLSSLTSEDSAVYYCARSSTWYYFDYWGQGTLVTVSAGGGGSDIQMTQTPSSLSASLGDRVTISCSASQDISNYLNWYQQKPDGTVKLLIYYTSRLHSGVPSRFSGSGSGTDYSLTISNLEPEDIATYYCQQGNTLPYTFGGGTKLEIKGGGASVASDPPVTITNPAT

SEQ ID NO: 19 - protein, 230 aa, Artificial sequence
  chimeric protein
QVQLQQSGAELVRPGTSVKISCKASGYTFTDYGMHWVKQRPGHGLEWIGIISTYSGDASYNQKFKGKATMTVDKSSSTAYMQLSSLTSEDSAVYYCARSSTWYYFDYWGQGTLVTVSAGGGGSDIQMTQTPSSLSASLGDRVTISCSASQDISNYLNWYQQKPDGTVKLLIYYTSRLHSGVPSRFSGSGSGTDYSLTISNLEPEDIATYYCQQGNTLPYTFGGGTKLEIK

SEQ ID NO: 20 - protein, 251 aa, Artificial sequence
  chimeric protein
QVQLQQSGAELVRPGTSVKISCKASGYTFTDYGMHWVKQRPGHGLEWIGIISTYSGDASYNQKFKGKATMTVDKSSSTAYMQLSSLTSEDSAVYYCARSSTWYYFDYWGQGTLVTVSAGGGGSGGGGSGGGGSDIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGASHHHHHH

SEQ ID NO: 21 - protein, 251 aa, Artificial sequence
  chimeric protein
QVQLQQSGAELVRPGTSVKISCKASGYTFTDYGMHWVKQSHAKSLEWIGIISTYSGDASYNQKFKGKATMTVDKSSSTAYMQLSSLTSEDSAVYYCARSSTWYYFDYWGQGTLVTVSAGGGGSGGGGSGGGGSDIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGASHHHHHH

SEQ ID NO: 22 - protein, 251 aa, Artificial sequence
  chimeric protein
QVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGGSGGGGSGGGGSDIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGASHHHHHH

SEQ ID NO: 23 - protein, 251 aa, Artificial sequence
  chimeric protein
QVQLQQSGAELVRPGVSVKISCKGSGYTFTDYGMHWVKQRPGHGLEWIGIISTYSGDASYNQKFKGKATMTVDKSSSTAYMELARLTSEDSAIYYCARSSTWYYFDYWGQGTTLTVSSGGGGSGGGGSGGGGSDIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGASHHHHHH

SEQ ID NO: 24 - protein, 251 aa, Artificial sequence
  chimeric protein
QVQLQQSGAEVKKPGASVKVSCKASGYTFTDYGMHWVRQAPGQGLEWMGIISTYSGDASYNQKFQGRVTMTRDTSTSTVYMELSSLRSEDTAVYYCARSSTWYYFDYWGQGTLVTVSSGGGGSGGGGSGGGGSDIQMTQSPSSLSASVGDRVTITCRASQDISNYLNWYQQKPGKAPKLLIYYTSRLHSGVPSRFSGSGSGTDFTLTISSLQPEDFATYYCQQGNTLPYTFGQGTKLEIKGGGASHHHHHH

SEQ ID NO: 25 - protein, 251 aa, Artificial sequence
  chimeric protein
DIQVTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKGGGGSGGGGSGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGASHHHHHH

SEQ ID NO: 26 - protein, 241 aa, Artificial sequence
  chimeric protein
DIQVTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGASHHHHHH

SEQ ID NO: 27 - protein, 251 aa, Artificial sequence
  chimeric protein
DIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKGGGGSGGGGSGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTLVTVSSGGGASHHHHHH

SEQ ID NO: 28 - protein, 241 aa, Artificial sequence
  chimeric protein
DIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTLVTVSSGGGASHHHHHH

SEQ ID NO: 29 - protein, 383 aa, Artificial sequence
  chimeric protein
DIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKGGGGSGGGGSGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTLVTVSSEPRGPTIKPCPPCKCGGGSSGGGSGGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGASHHHHHH

SEQ ID NO: 30 - protein, 373 aa, Artificial sequence
  chimeric protein
DIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTLVTVSSEPRGPTIKPCPPCKCGGGSSGGGSGGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGASHHHHHH

SEQ ID NO: 31 - protein, 484 aa, Artificial sequence
  chimeric protein
DIQVTQTPSSLSASLGDRVTISCRASQDISNYLNWYQQKPDGTVKLLIYYTSRLHSGVPSRFSGSGSGTDYSLTISNLEQEDIATYYCQQGNTLPYTFGGGTKLEIKGGGGSGGGGSGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTLVTVSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGASHHHHHH

SEQ ID NO: 32 - protein, 251 aa, Artificial sequence
  chimeric protein
DIQVTQTPSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKGGGGSGGGGSGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGASHHHHHH

SEQ ID NO: 33 - protein, 249 aa, Artificial sequence
  chimeric protein
DIQMTQTPSSLSASLGDRVTISCSASQDISNYLNWYQQKPDGTVKLLIYYTSRLHSGVPSRFSGSGSGTDYSLTISNLEPEDIATYYCQQGNTLPYTFGGGTKLEIKGGGGSQVQLQQSGAELVRPGTSVKISCKASGYTFTDYGMHWVKQRPGHGLEWIGIISTYSGDASYNQKFKGKATMTVDKSSSTAYMQLSSLTSEDSAVYYCARSSTWYYFDYWGQGTLVTVSAGGGASVASDPPVTITNPAT

SEQ ID NO: 34 - protein, 230 aa, Artificial sequence
  chimeric protein
DIQMTQTPSSLSASLGDRVTISCSASQDISNYLNWYQQKPDGTVKLLIYYTSRLHSGVPSRFSGSGSGTDYSLTISNLEPEDIATYYCQQGNTLPYTFGGGTKLEIKGGGGSQVQLQQSGAELVRPGTSVKISCKASGYTFTDYGMHWVKQRPGHGLEWIGIISTYSGDASYNQKFKGKATMTVDKSSSTAYMQLSSLTSEDSAVYYCARSSTWYYFDYWGQGTLVTVSA

SEQ ID NO: 35 - protein, 251 aa, Artificial sequence
  chimeric protein
DIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGGSGGGGSGGGGSQVQLQQSGAELVRPGTSVKISCKASGYTFTDYGMHWVKQRPGHGLEWIGIISTYSGDASYNQKFKGKATMTVDKSSSTAYMQLSSLTSEDSAVYYCARSSTWYYFDYWGQGTLVTVSAGGGASHHHHHH

SEQ ID NO: 36 - protein, 251 aa, Artificial sequence
  chimeric protein
DIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGGSGGGGSGGGGSQVQLQQSGAELVRPGTSVKISCKASGYTFTDYGMHWVKQSHAKSLEWIGIISTYSGDASYNQKFKGKATMTVDKSSSTAYMQLSSLTSEDSAVYYCARSSTWYYFDYWGQGTLVTVSAGGGASHHHHHH

SEQ ID NO: 37 - protein, 251 aa, Artificial sequence
  chimeric protein
DIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGGSGGGGSGGGGSQVQLQQSGAELVRPGVSVKISCKGSGYTFTDYGMHWVKQSHAKSLEWIGIISTYSGDASYNQKFKGKATMTVDKSSSTAYMELARLTSEDSAIYYCARSSTWYYFDYWGQGTTLTVSSGGGASHHHHHH

SEQ ID NO: 38 - protein, 251 aa, Artificial sequence
  chimeric protein
DIQMTQTPSSLSASLGDRVTISCRASQDISNYLNWYQQKPDGTVKLLIYYTSRLHSGVPSRFSGSGSGTDYSLTISNLEPEDIATYYCQQGNTLPYTFGGGTKLEIKGGGGSGGGGSGGGGSQVQLQQSGAELVRPGVSVKISCKGSGYTFTDYGMHWVKQRPGHGLEWIGIISTYSGDASYNQKFKGKATMTVDKSSSTAYMELARLTSEDSAIYYCARSSTWYYFDYWGQGTTLTVSSGGGASHHHHHH

SEQ ID NO: 39 - protein, 251 aa, Artificial sequence
  chimeric protein
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPYTFGQGTKLEIKGGGGSGGGGSGGGGSQVQLQQSGAEVKKPGASVKVSCKASGYTFTDYGMHWVRQAPGQGLEWMGIISTYSGDASYNQKFQGRVTMTRDTSTSTVYMELSSLRSEDTAVYYCARSSTWYYFDYWGQGTLVTVSSGGGASHHHHHH

SEQ ID NO: 40 - DNA, 753 bp, Artificial sequence
  synthetic gene
caggttcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggcggatccggtggcggtggctccggtggcggtggcagtgatattcaggttacccagaccacctctagcctgagcgcaagcctgggtgatcgtgttaccattagctgtcgtgcaagccaggatattagcaattatctgaattggtatcagcagaaaccggatggcaccgttaaactgctgatttattataccagccgtctgcatagcggtgttccgagccgttttagcggtagcggttctggcaccgattatagcctgaccatttctaatctggaacaggaagatattgccacctatttttgccagcagggtaatacactgccgtatacctttggtggaggcaccaaactggaaattaaaggcggtggtgctagccatcaccatcaccatcac

SEQ ID NO: 41 - DNA, 723 bp, Artificial sequence
  synthetic gene
caggttcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggcggatccgatattcaggttacccagaccacctctagcctgagcgcaagcctgggtgatcgtgttaccattagctgtcgtgcaagccaggatattagcaattatctgaattggtatcagcagaaaccggatggcaccgttaaactgctgatttattataccagccgtctgcatagcggtgttccgagccgttttagcggtagcggttctggcaccgattatagcctgaccatttctaatctggaacaggaagatattgccacctatttttgccagcagggtaatacactgccgtatacctttggtggaggcaccaaactggaaattaaaggcggtggtgctagccatcaccatcaccatcac

SEQ ID NO: 42 - DNA, 753 bp, Artificial sequence
  synthetic gene
caggtgcagctgcagcagagcggagccgagctcgtgcgcccaggcgtgtccgtgaagattagctgcaagggcagcggctacaccttcaccgattacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcaccaccctgacggtgtccagcggaggcggaggaagtggcggcggaggatcgggcggaggcggcagtgatattcaagtgacccagacccccagcagcctgagcgccagtctgggagatcgcgtgaccattagttgccgcgccagccaggacatcagcaactacctgaactggtatcagcagaagcccgatggcaccgtgaagctgctgatctactacaccagccgcctgcacagcggagtgccaagccgctttagtggcagcggcagcggaaccgattacagcctgaccatctccaacctggagcaggaggatattgccacctactactgccagcagggcaataccctgccctacacctttggcggcggtaccaagctggagatcaagggcggaggcgctagccaccaccaccatcaccac

SEQ ID NO: 43 - DNA, 723 bp, Artificial sequence
  synthetic gene
caggtgcagctgcagcagagcggagccgagctcgtgcgcccaggcgtgtccgtgaagattagctgcaagggcagcggctacaccttcaccgattacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcaccaccctgacggtgtccagcggaggcggaggcagcgatattcaagtgacccagacccccagcagcctgagcgccagtctgggagatcgcgtgaccattagttgccgcgccagccaggacatcagcaactacctgaactggtatcagcagaagcccgatggcaccgtgaagctgctgatctactacaccagccgcctgcacagcggagtgccaagccgctttagtggcagcggcagcggaaccgattacagcctgaccatctccaacctggagcaggaggatattgccacctactactgccagcagggcaataccctgccctacacctttggcggcggtaccaagctggagatcaagggcggaggcgctagccaccaccaccatcaccac

SEQ ID NO: 44 - DNA, 1149 bp, Artificial sequence
  synthetic gene
caggtgcagctgcagcagagcggagccgagctcgtgcgcccaggcgtgtccgtgaagattagctgcaagggcagcggctacaccttcaccgattacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcaccaccctgacggtgtccagcggaggcggaggaagtggcggcggaggatcgggcggaggcggcagtgatattcaagtgacccagacccccagcagcctgagcgccagtctgggagatcgcgtgaccattagttgccgcgccagccaggacatcagcaactacctgaactggtatcagcagaagcccgatggcaccgtgaagctgctgatctactacaccagccgcctgcacagcggagtgccaagccgctttagtggcagcggcagcggaaccgattacagcctgaccatctccaacctggagcaggaggatattgccacctactactgccagcagggcaataccctgccctacacctttggcggcggtaccaagctggagatcaaggagccacgcggcccaaccatcaagccctgcccaccatgcaagtgcggaggcggaagtagcggaggcggctccggcggaagtgtgcgcgccccacaggtgtacgtgctgccaccaccagaggaggagatgaccaagaagcaagtgacgctgacctgcatggtcaccgatttcatgcccgaggacatctacgtggagtggaccaacaacggcaagaccgagctgaactacaagaacaccgagcccgtgctggattccgatggcagctacttcatgtacagcaagctgcgcgtggagaagaagaactgggtggagcgcaacagctacagctgctccgtggtgcacgagggcctgcacaatcaccacaccaccaagagcttcagccgcaccccaggcaagggcggaggcgctagccaccaccaccatcaccac

SEQ ID NO: 45 - DNA, 1119 bp, Artificial sequence
  synthetic gene
caggtgcagctgcagcagagcggagccgagctcgtgcgcccaggcgtgtccgtgaagattagctgcaagggcagcggctacaccttcaccgattacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcaccaccctgacggtgtccagcggaggcggaggcagcgatattcaagtgacccagacccccagcagcctgagcgccagtctgggagatcgcgtgaccattagttgccgcgccagccaggacatcagcaactacctgaactggtatcagcagaagcccgatggcaccgtgaagctgctgatctactacaccagccgcctgcacagcggagtgccaagccgctttagtggcagcggcagcggaaccgattacagcctgaccatctccaacctggagcaggaggatattgccacctactactgccagcagggcaataccctgccctacacctttggcggcggtaccaagctggagatcaaggagccacgcggcccaaccatcaagccctgcccaccatgcaagtgcggaggcggaagtagcggaggcggctccggcggaagtgtgcgcgccccacaggtgtacgtgctgccaccaccagaggaggagatgaccaagaagcaagtgacgctgacctgcatggtcaccgatttcatgcccgaggacatctacgtggagtggaccaacaacggcaagaccgagctgaactacaagaacaccgagcccgtgctggattccgatggcagctacttcatgtacagcaagctgcgcgtggagaagaagaactgggtggagcgcaacagctacagctgctccgtggtgcacgagggcctgcacaatcaccacaccaccaagagcttcagccgcaccccaggcaagggcggaggcgctagccaccaccaccatcaccac

SEQ ID NO: 46 - DNA, 1452 bp, Artificial sequence
  synthetic gene
caggtgcagctgcagcagagcggagccgagctcgtgcgcccaggcgtgtccgtgaagattagctgcaagggcagcggctacaccttcaccgattacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcaccaccctgacggtgtccagcggaggcggaggaagtggcggcggaggatcgggcggaggcggcagtgatattcaagtgacccagacccccagcagcctgagcgccagtctgggagatcgcgtgaccattagttgccgcgccagccaggacatcagcaactacctgaactggtatcagcagaagcccgatggcaccgtgaagctgctgatctactacaccagccgcctgcacagcggagtgccaagccgctttagtggcagcggcagcggaaccgattacagcctgaccatctccaacctggagcaggaggatattgccacctactactgccagcagggcaataccctgccctacacctttggcggcggtaccaagctggagatcaaggagccacgcggcccaaccatcaagccctgcccaccatgcaagtgcccagcccccaatctgctgggaggccccagcgtgttcatcttcccgcccaagattaaggacgtgctgatgatcagcctgagccccatcgtgacctgcgtggtggtggatgtgtccgaggatgatcccgatgtgcagatcagttggttcgtgaacaacgtggaggtgcacaccgcccagacccagacgcaccgcgaggattacaatagcaccctgcgcgtggtgtccgccctgccaattcagcaccaggattggatgagcggcaaggagttcaagtgcaaagtgaacaacaaggatctgccagccccgatcgagcgcaccatcagcaagccaaagggaagcgtgcgcgccccccaggtgtacgtgctgccaccaccagaggaggagatgaccaagaagcaagtgaccctgacctgcatggtcaccgatttcatgcccgaggacatctacgtggagtggaccaacaacggcaagaccgagctgaactacaagaacaccgagcccgtgctggattccgatggcagctacttcatgtacagcaagctgcgcgtggagaagaagaactgggtggagcgcaacagctacagctgctccgtggtgcacgagggcctgcacaatcaccacaccaccaagagcttcagccgcaccccaggcaagggcggaggcgctagccaccatcatcaccaccac

SEQ ID NO: 47 - DNA, 753 bp, Artificial sequence
  synthetic gene
caggtgcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggcggatccggtggcggtggctccggtggcggtggcagtgatattcaggttacccagaccccgtctagcctgagcgcaagcctgggtgatcgtgttaccattagctgtcgtgcaagccaggatattagcaattatctgaattggtatcagcagaaaccggatggcaccgttaaactgctgatttattataccagccgtctgcatagcggtgttccgagccgttttagcggtagcggttctggcaccgattatagcctgaccatttctaatctggaacaggaagatattgccacctatttttgccagcagggtaatacactgccgtatacctttggtggaggcaccaaactggaaattaaaggcggtggtgctagccatcaccatcaccatcac

SEQ ID NO: 48 - DNA, 747 bp, Artificial sequence
  synthetic gene
caggtgcagctgcagcagagcggtgcagaactggttcgtccgggtacaagcgtgaaaatttcatgtaaagcaagcggctataccttcaccgattatggtatgcattgggttaaacagcgtccgggtcatggtctggaatggattggtattattagcacctatagcggtgatgccagctataaccaaaaattcaaaggtaaagcaaccatgaccgtagataaaagcagcagcaccgcctatatgcagctgagcagcctgaccagcgaagatagcgcagtttattactgtgcacgtagcagcacctggtattactttgattattggggtcagggcaccctggttaccgttagtgccggtggtggtggatccgatattcagatgacccagaccccgagcagtctgagcgcaagcctgggtgatcgtgttaccattagctgtagcgccagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggcgctagcgttgcaagcgatcctccggttacaattaccaatccggcaacc

SEQ ID NO: 49 - DNA, 690 bp, Artificial sequence
  gene synthetique
caggtgcagctgcagcagagcggtgcagaactggttcgtccgggtacaagcgtgaaaatttcatgtaaagcaagcggctataccttcaccgattatggtatgcattgggttaaacagcgtccgggtcatggtctggaatggattggtattattagcacctatagcggtgatgccagctataaccaaaaattcaaaggtaaagcaaccatgaccgtagataaaagcagcagcaccgcctatatgcagctgagcagcctgaccagcgaagatagcgcagtttattactgtgcacgtagcagcacctggtattactttgattattggggtcagggcaccctggttaccgttagtgccggtggtggtggatccgatattcagatgacccagaccccgagcagtctgagcgcaagcctgggtgatcgtgttaccattagctgtagcgccagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaa

SEQ ID NO: 50 - DNA, 753 bp, Artificial sequence
  synthetic gene
caggtgcagctgcagcagagcggtgcagaactggttcgtccgggtacaagcgtgaaaatttcatgtaaagcaagcggctataccttcaccgattatggtatgcattgggttaaacagcgtccgggtcatggtctggaatggattggtattattagcacctatagcggtgatgccagctataaccaaaaattcaaaggtaaagcaaccatgaccgtagataaaagcagcagcaccgcctatatgcagctgagcagcctgaccagcgaagatagcgcagtttattactgtgcacgtagcagcacctggtattactttgattattggggtcagggcaccctggttaccgttagtgccggtggtggtggatccggtggcggtggtagcggaggtggtggtagtgatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggcgctagccatcatcatcaccatcat

SEQ ID NO: 51 - DNA, 753 bp, Artificial sequence
  synthetic gene
caggtgcagctgcagcagagcggtgcagaactggttcgtccgggtacaagcgtgaaaatttcatgtaaagcaagcggctataccttcaccgattatggtatgcattgggttaaacagagccatgcaaaaagcctggaatggattggtattattagcacctatagcggtgatgccagctataaccagaaattcaaaggtaaagcaaccatgaccgttgataaaagcagcagcaccgcctatatgcagctgagcagcctgaccagcgaagatagcgcagtttattactgtgcacgtagcagcacctggtattactttgattattggggtcagggtacactagtcaccgttagtgccggtggtggtggatccggtggcggtggtagcggaggtggtggtagtgatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggcgctagccatcatcatcaccatcat

SEQ ID NO: 52 - DNA, 753 bp, Artificial sequence
  synthetic gene
caggtgcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggcggatccggtggcggtggtagcggaggtggtggtagtgatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggcgctagccatcatcatcaccatcat

SEQ ID NO: 53 - DNA, 753 bp, Artificial sequence
  synthetic gene
caggtgcagctgcagcagagcggtgcagagctggttcgtccgggtgttagcgttaaaattagctgcaaaggtagcggctatacctttaccgattatggtatgcattgggtgaaacagcgcccgggtcatggcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggcggatccggtggcggtggtagcggaggtggtggtagtgatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggcgctagccatcatcatcaccatcat

SEQ ID NO: 54 - DNA, 753 bp, Artificial sequence
  synthetic gene
caggtgcagctgcagcagagcggtgccgaagttaaaaaaccgggtgcaagcgttaaagttagctgtaaagcaagcggttatacctttaccgattatggtatgcactgggttcgtcaggcaccgggtcagggtctggaatggatgggtattattagcacctatagcggtgatgcaagctacaatcagaaatttcagggtcgtgttaccatgacccgtgataccagcaccagcaccgtttatatggaactgagcagcctgcgtagcgaagataccgcagtgtattattgtgcacgtagcagcacctggtattatttcgattattggggtcagggcaccctggttaccgttagcagcggtggtggtggatccggtggtggcggttcaggtggcggtggtagcgatattcagatgacccagagcccgagcagtctgagcgcaagcgttggtgatcgtgtgaccattacctgtcgtgcaagccaggatattagcaattatctgaattggtatcagcagaaaccgggtaaagcaccgaaactgctgatctattataccagccgtctgcatagcggtgttccgagccgttttagcggtagcggtagtggcaccgattttaccctgaccattagcagtctgcaaccggaagattttgcaacctattattgtcagcagggcaataccctgccgtatacctttggccagggcaccaaactggaaattaaaggcggtggtgctagccatcatcatcaccatcat

SEQ ID NO: 55 - DNA, 753 bp, Artificial sequence
  synthetic gene
gatattcaggttacccagaccacctctagcctgagcgcaagcctgggtgatcgtgttaccattagctgtcgtgcaagccaggatattagcaattatctgaattggtatcagcagaaaccggatggcaccgttaaactgctgatttattataccagccgtctgcatagcggtgttccgagccgttttagcggtagcggttctggcaccgattatagcctgaccatttctaatctggaacaggaagatattgccacctatttttgccagcagggtaatacactgccgtatacctttggtggaggcaccaaactggaaattaaaggcggtggcggatccggtggcggtggctccggtggcggtggcagtcaggttcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggtgctagccatcaccatcaccatcac

SEQ ID NO: 56 - DNA, 723 bp, Artificial sequence
  synthetic gene
gatattcaggttacccagaccacctctagcctgagcgcaagcctgggtgatcgtgttaccattagctgtcgtgcaagccaggatattagcaattatctgaattggtatcagcagaaaccggatggcaccgttaaactgctgatttattataccagccgtctgcatagcggtgttccgagccgttttagcggtagcggttctggcaccgattatagcctgaccatttctaatctggaacaggaagatattgccacctatttttgccagcagggtaatacactgccgtatacctttggtggaggcaccaaactggaaattaaaggcggtggcggatcccaggttcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggtgctagccatcaccatcaccatcac

SEQ ID NO: 57 - DNA, 753 bp, Artificial sequence
  synthetic gene
gatatccaagtgacccagacccccagcagcctgagtgccagtctgggagatcgcgtgaccattagctgccgcgccagccaggacatcagcaactacctgaactggtatcagcagaagcccgatggcaccgtgaagctgctgatctactacaccagccgcctgcacagcggagtgccaagccgctttagtggcagcggctccggcaccgattacagcctgaccatctccaacctggagcaggaggatattgccacctactactgccagcagggcaacaccctgccctacacctttggcggaggcaccaagctggagatcaagggcggaggcggaagtggcggcggaggaagcggaggcggcggaagccaggtgcagttgcagcagagcggagccgagctcgtgcgcccaggcgtgtccgtgaagattagctgcaagggctccggctacaccttcaccgactacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcacactagtcaccgtgtccagtggcggaggcgctagccaccaccaccatcaccac

SEQ ID NO: 58 - DNA, 722 bp, Artificial sequence
  synthetic gene
gatatccaagtgacccagacccccagcagcctgagtgccagtctgggagatcgcgtgaccattagctgccgcgccagccaggacatcagcaactacctgaactggtatcagcagaagcccgatggcaccgtgaagctgctgatctactacaccagccgcctgcacagcggagtgccaagccgctttagtggcagcggctccggcaccgattacagcctgaccatctccaacctggagcaggaggatattgccacctactactgccagcagggcaacaccctgccctacacctttggcggaggcaccaagctggagatcaaggaggcggaggcagccaggtgcagttgcagcagagcggagccgagctcgtgcgcccaggcgtgtccgtgaagattagctgcaagggctccggctacaccttcaccgactacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcacactagtcaccgtgtccagtggcggaggcgctagccaccaccaccatcaccac

SEQ ID NO: 59 - DNA, 1149 bp, Artificial sequence
  synthetic gene
gatatccaagtgacccagacccccagcagcctgagtgccagtctgggagatcgcgtgaccattagctgccgcgccagccaggacatcagcaactacctgaactggtatcagcagaagcccgatggcaccgtgaagctgctgatctactacaccagccgcctgcacagcggagtgccaagccgctttagtggcagcggctccggcaccgattacagcctgaccatctccaacctggagcaggaggatattgccacctactactgccagcagggcaacaccctgccctacacctttggcggaggcaccaagctggagatcaagggcggaggcggaagtggcggcggaggaagcggaggcggcggaagccaggtgcagttgcagcagagcggagccgagctcgtgcgcccaggcgtgtccgtgaagattagctgcaagggctccggctacaccttcaccgactacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcacactagtcaccgtgtccagtgagccacgcggcccaaccatcaagccctgcccaccatgcaagtgcggaggcggaagtagcggaggcggctccggcggaagtgtgcgcgccccacaggtgtacgtgctgccaccaccagaggaggagatgaccaagaagcaagtgacgctgacctgcatggtcaccgatttcatgcccgaggacatctacgtggagtggaccaacaacggcaagaccgagctgaactacaagaacaccgagcccgtgctggattccgatggcagctacttcatgtacagcaagctgcgcgtggagaagaagaactgggtggagcgcaacagctacagctgctccgtggtgcacgagggcctgcacaatcaccacaccaccaagagcttcagccgcaccccaggcaagggcggaggcgctagccaccaccaccatcaccac

SEQ ID NO: 60 - DNA, 1118 bp, Artificial sequence
  synthetic gene
gatatccaagtgacccagacccccagcagcctgagtgccagtctgggagatcgcgtgaccattagctgccgcgccagccaggacatcagcaactacctgaactggtatcagcagaagcccgatggcaccgtgaagctgctgatctactacaccagccgcctgcacagcggagtgccaagccgctttagtggcagcggctccggcaccgattacagcctgaccatctccaacctggagcaggaggatattgccacctactactgccagcagggcaacaccctgccctacacctttggcggaggcaccaagctggagatcaaggaggcggaggcagccaggtgcagttgcagcagagcggagccgagctcgtgcgcccaggcgtgtccgtgaagattagctgcaagggctccggctacaccttcaccgactacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcacactagtcaccgtgtccagtgagccacgcggcccaaccatcaagccctgcccaccatgcaagtgcggaggcggaagtagcggaggcggctccggcggaagtgtgcgcgccccacaggtgtacgtgctgccaccaccagaggaggagatgaccaagaagcaagtgacgctgacctgcatggtcaccgatttcatgcccgaggacatctacgtggagtggaccaacaacggcaagaccgagctgaactacaagaacaccgagcccgtgctggattccgatggcagctacttcatgtacagcaagctgcgcgtggagaagaagaactgggtggagcgcaacagctacagctgctccgtggtgcacgagggcctgcacaatcaccacaccaccaagagcttcagccgcaccccaggcaagggcggaggcgctagccaccaccaccatcaccac

SEQ ID NO: 61 - DNA, 1452 bp, Artificial sequence
  synthetic gene
gatatccaagtgacccagacccccagcagcctgagtgccagtctgggagatcgcgtgaccattagctgccgcgccagccaggacatcagcaactacctgaactggtatcagcagaagcccgatggcaccgtgaagctgctgatctactacaccagccgcctgcacagcggagtgccaagccgctttagtggcagcggctccggcaccgattacagcctgaccatctccaacctggagcaggaggatattgccacctactactgccagcagggcaacaccctgccctacacctttggcggaggcaccaagctggagatcaagggcggaggcggaagtggcggcggaggaagcggaggcggcggaagccaggtgcagttgcagcagagcggagccgagctcgtgcgcccaggcgtgtccgtgaagattagctgcaagggctccggctacaccttcaccgactacggcatgcactgggtcaagcagagccacgccaagagcctggagtggatcggcatcatcagcacctacagcggcgacgccagctacaaccagaagttcaagggcaaggccaccatgaccgtggataagagcagcagcaccgcctacatggagctggcccgcctgaccagcgaggatagcgccatctattactgcgcccgcagctccacctggtactacttcgattactggggccagggcacactagtcaccgtgtccagtgagccacgcggcccaaccatcaagccctgcccaccatgcaagtgcccagcccccaatctgctgggaggccccagcgtgttcatcttcccgcccaagattaaggacgtgctgatgatcagcctgagccccatcgtgacctgcgtggtggtggatgtgtccgaggatgatcccgatgtgcagatcagttggttcgtgaacaacgtggaggtgcacaccgcccagacccagacgcaccgcgaggattacaatagcaccctgcgcgtggtgtccgccctgccaattcagcaccaggattggatgagcggcaaggagttcaagtgcaaagtgaacaacaaggatctgccagccccgatcgagcgcaccatcagcaagccaaagggaagcgtgcgcgccccccaggtgtacgtgctgccaccaccagaggaggagatgaccaagaagcaagtgaccctgacctgcatggtcaccgatttcatgcccgaggacatctacgtggagtggaccaacaacggcaagaccgagctgaactacaagaacaccgagcccgtgctggattccgatggcagctacttcatgtacagcaagctgcgcgtggagaagaagaactgggtggagcgcaacagctacagctgctccgtggtgcacgagggcctgcacaatcaccacaccaccaagagcttcagccgcaccccaggcaagggcggaggcgctagccaccaccaccatcaccac

SEQ ID NO: 62 - DNA, 753 bp, Artificial sequence
  synthetic gene
gatattcaggttacccagaccccgtctagcctgagcgcaagcctgggtgatcgtgttaccattagctgtcgtgcaagccaggatattagcaattatctgaattggtatcagcagaaaccggatggcaccgttaaactgctgatttattataccagccgtctgcatagcggtgttccgagccgttttagcggtagcggttctggcaccgattatagcctgaccatttctaatctggaacaggaagatattgccacctatttttgccagcagggtaatacactgccgtatacctttggtggaggcaccaaactggaaattaaaggcggtggcggatccggtggcggtggctccggtggcggtggcagtcaggtgcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggcggtggtgctagccatcaccatcaccatcac

SEQ ID NO: 63 - DNA, 747 bp, Artificial sequence
  synthetic gene
gatattcagatgacccagaccccgagcagtctgagcgcaagcctgggtgatcgtgttaccattagctgtagcgccagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggtggatcccaggtgcagctgcagcagagcggtgcagaactggttcgtccgggtacaagcgtgaaaatttcatgtaaagcaagcggctataccttcaccgattatggtatgcattgggttaaacagcgtccgggtcatggtctggaatggattggtattattagcacctatagcggtgatgccagctataaccaaaaattcaaaggtaaagcaaccatgaccgtagataaaagcagcagcaccgcctatatgcagctgagcagcctgaccagcgaagatagcgcagtttattactgtgcacgtagcagcacctggtattactttgattattggggtcagggcaccctggttaccgttagtgccggtggtggcgctagcgttgcaagcgatcctccggttacaattaccaatccggcaacc

SEQ ID NO: 64 - DNA, 690 bp, Artificial sequence
  synthetic gene
gatattcagatgacccagaccccgagcagtctgagcgcaagcctgggtgatcgtgttaccattagctgtagcgccagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggtggatcccaggtgcagctgcagcagagcggtgcagaactggttcgtccgggtacaagcgtgaaaatttcatgtaaagcaagcggctataccttcaccgattatggtatgcattgggttaaacagcgtccgggtcatggtctggaatggattggtattattagcacctatagcggtgatgccagctataaccaaaaattcaaaggtaaagcaaccatgaccgtagataaaagcagcagcaccgcctatatgcagctgagcagcctgaccagcgaagatagcgcagtttattactgtgcacgtagcagcacctggtattactttgattattggggtcagggcaccctggttaccgttagtgcc

SEQ ID NO: 65 - DNA, 753 bp, Artificial sequence
  synthetic gene
gatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggtggatccggtggcggtggtagcggaggtggtggtagtcaggtgcagctgcagcagagcggtgcagaactggttcgtccgggtacaagcgtgaaaatttcatgtaaagcaagcggctataccttcaccgattatggtatgcattgggttaaacagcgtccgggtcatggtctggaatggattggtattattagcacctatagcggtgatgccagctataaccaaaaattcaaaggtaaagcaaccatgaccgtagataaaagcagcagcaccgcctatatgcagctgagcagcctgaccagcgaagatagcgcagtttattactgtgcacgtagcagcacctggtattactttgattattggggtcagggcaccctggttaccgttagtgccggtggtggcgctagccatcatcatcaccatcat

SEQ ID NO: 66 - DNA, 753 bp, Artificial sequence
  synthetic gene
gatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggtggtggtggatccggtggcggtggtagcggaggtggtggtagtcaggtgcagctgcagcagagcggtgcagaactggttcgtccgggtacaagcgtgaaaatttcatgtaaagcaagcggctataccttcaccgattatggtatgcattgggttaaacagagccatgcaaaaagcctggaatggattggtattattagcacctatagcggtgatgccagctataaccagaaattcaaaggtaaagcaaccatgaccgttgataaaagcagcagcaccgcctatatgcagctgagcagcctgaccagcgaagatagcgcagtttattactgtgcacgtagcagcacctggtattactttgattattggggtcagggtacactagtcaccgttagtgccggtggtggcgctagccatcatcatcaccatcat

SEQ ID NO: 67 - DNA, 753 bp, Artificial sequence
  synthetic gene
gatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggcggtggcggatccggtggcggtggtagcggaggtggtggtagtcaggtgcagctgcagcagagcggtgcagagctggttcgtccgggtgttagcgttaaaattagctgcaaaggtagcggctatacctttaccgattatggtatgcattgggtgaaacagagccatgcaaaaagcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggtggtggcgctagccatcatcatcaccatcat

SEQ ID NO: 68 - DNA, 753 bp, Artificial sequence
  synthetic gene
gatatccagatgacccagaccccgagcagtctgagcgcaagcctgggtgatcgtgttaccattagctgtcgtgcaagccaggatattagcaattatctgaattggtatcagcagaaaccggatggcaccgttaaactgctgatctattataccagccgtctgcatagcggtgttccgagccgttttagcggtagcggttcaggtacagattatagtctgaccattagcaatctggaaccggaagatattgccacctattattgtcagcagggcaataccctgccgtatacctttggtggtggcaccaaactggaaattaaaggcggtggcggatccggtggcggtggtagcggaggtggtggtagtcaggtgcagctgcagcagagcggtgcagagctggttcgtccgggtgttagcgttaaaattagctgcaaaggtagcggctatacctttaccgattatggtatgcattgggtgaaacagcgcccgggtcatggcctggaatggattggtattattagcacctatagcggtgatgcaagctataatcagaaatttaaaggcaaagccaccatgaccgttgataaaagcagcagcaccgcatatatggagctggctcgtctgaccagcgaagatagcgccatttattactgtgcacgtagcagcacctggtattattttgattattggggtcagggcaccacactgaccgttagctcaggtggtggcgctagccatcatcatcaccatcat

SEQ ID NO: 69 - DNA, 753 bp, Artificial sequence
  synthetic gene
gatattcagatgacccagagcccgagcagtctgagcgcaagcgttggtgatcgtgtgaccattacctgtcgtgcaagccaggatattagcaattatctgaattggtatcagcagaaaccgggtaaagcaccgaaactgctgatctattataccagccgtctgcatagcggtgttccgagccgttttagcggtagcggtagtggcaccgattttaccctgaccattagcagtctgcaaccggaagattttgcaacctattattgtcagcagggcaataccctgccgtatacctttggccagggcaccaaactggaaattaaaggtggtggtggatccggtggtggcggttcaggtggcggtggtagccaggtgcagctgcagcagagcggtgccgaagttaaaaaaccgggtgcaagcgttaaagttagctgtaaagcaagcggttatacctttaccgattatggtatgcactgggttcgtcaggcaccgggtcagggtctggaatggatgggtattattagcacctatagcggtgatgcaagctacaatcagaaatttcagggtcgtgttaccatgacccgtgataccagcaccagcaccgtttatatggaactgagcagcctgcgtagcgaagataccgcagtgtattattgtgcacgtagcagcacctggtattatttcgattattggggtcagggcaccctggttaccgttagcagcggcggtggtgctagccatcatcatcaccatcat

SEQ ID NO: 70 - DNA, 24 bp, Artificial sequence
  primer SG5For
ggatcccgatattcaggttaccag

SEQ ID NO: 71 - DNA, 24 bp, Artificial sequence
  primer SG5Rev
tttctcgagttagtgatggtgatg

(19) United States
(12) Patent Application Publication
(10) Pub. No.: US 2019/0153083 A1
(43) Pub. Date: May 23, 2019

(54) NOVEL PEPTIDE STRUCTURES AND USE THEREOF IN THE TREATMENT OF TOXOPLASMOSIS

(71) Applicant: UNIVERSITE FRANCOIS RABELAIS, Tours (FR)

(72) Inventors: Matthieu JUSTE, Poitiers (FR); Isabelle DIMIER-POISSON, Tours (FR); Nicolas AUBREY, Truyes (FR); Anne DI TOMMASO, La Riche (FR)

(21) Appl. No.: 16/093,215

(22) PCT Filed: Apr. 11, 2017
(86) PCT No.: PCT/FR2017/050875

§ 371 Date: Oct. 12, 2018

(30) Foreign Application Priority Data
Apr. 12, 2016 (FR) 16 53206

(51) Int. Cl. C07K 16/20; A61P 33/02; C12N 15/62

(52) U.S. Cl. CPC C07K 16/20; A61P 33/02; C07K 2317/622; C07K 2317/76; C07K 2317/565; C12N 15/62

(57) ABSTRACT

The invention relates to a peptide structure which does not include a CH1 region, which recognises the SAG1 antigen of Toxoplasma gondii and which can neutralize the invasion of cells by Toxoplasma gondii, and to the use of said structure in the treatment of toxoplasmosis, in particular ocular toxoplasmosis, congenital toxoplasmosis and behavioural disorders linked to the presence of Toxoplasma gondii.

The invention relates to new peptide constructs recognizing the SAG1 antigen of Toxoplasma gondii and their use in the treatment of toxoplasmosis.

Since the High Antiquity, the men know the symptoms of the infectious diseases and today these still constitute a major “challenge” for the Biomedical Sciences. They remain one of the leading causes of morbidity and mortality in the world (33% of deaths in developing countries). Toxoplasmosis is one of these major societal challenges. Cosmopolitan parasitic disease caused by an obligate intracellular protozoan (Toxoplasma gondii), it remains one of those diseases having a strong economic and health impact, that one takes into consideration the Human Health or the Veterinary Health.

Although generally asymptomatic in humans, this disease can have a very severe character in the form of neurotoxoplasmosis, toxoplasmic chorioretinitis or congenital toxoplasmosis, which causes psychomotor disorders and/or ocular in children or, in the worst case, the cause of the loss of the unborn child following an abortion. In France, there are an estimated 800 000 patients with active or scarred ocular toxoplasmosis, related to the proliferation of Toxoplasma gondii in retinal tissues (Sauer et al., Ocular toxoplasmosis: from pathophysiology to microbiological diagnosis, J Fr Ophtalmol., 2013, vol 36, No. 1, pp 76-81, Plever et al., Ocular Toxoplasmosis: Recent Aspects of Pathophysiology and Clinical Implications, Ophthalmic Res., 2014, Vol 52, No. 3, pp 116-123. Maenz et al., Ocular toxoplasmosis past, present and new aspects of an old disease, Prog. Retin. Eye Res., 2014, vol 39, pp 77-106). Congenital toxoplasmosis (Hampton, Congenital Toxoplasmosis: A Review, Neonatal Network, 2015, vol 34, no. 5, pp 274-278), for its part, represents about 300 cases/year in France, following the transmission of the parasite from the mother to the fetus (CNR Toxoplasmosis, Annual Activity Report of the National Toxoplasmosis Reference Center, 2013). The parasite Toxoplasma gondii may also cause behavioral disturbances (Flegr, Schizophrenia and Toxoplasma gondii: an undervalued association?, Expert Rev. Anti Infect. Ther., 2015, Vol 13, No. 7, pp 817-820).

At the prophylactic level, no vaccine for human use is currently available, despite strong demand. In veterinary medicine, a live attenuated vaccine obtained from strain S48 is marketed (Ovilis®, Toxovax®, Intervet). In terms of curative solutions, treatments exist. Nevertheless, their effectiveness remains debatable, especially since they prove to be extremely painful for the patient, in terms of surveillance and adverse effects.

Although commonly used, spiramycin, sulfadiazine, azithromycin and pyrimethamine are constraining treatments in terms of monitoring (Blood Formula—Platelet Count) and side effects (Lyell epidermolysis syndrome, agranulocytosis, membranous pseudo-colitis). In addition, resistance to antiprotozoal inhibitors of dihydrofolate reductase (DHFR), such as pyrimethamine, can be observed in case of mutation in the gene encoding DHFR. In addition, this target is also present naturally in humans, this may lead to adverse effects (anemia, thrombocytopenia, granulopenia) and the concomitant administration of folic acid to offset these deleterious effects.

There is therefore a real need to develop an alternative therapeutic strategy for the use of the treatments currently used in the management of toxoplasmic chorioretinitis and congenital toxoplasmosis.

The use of therapeutic antibodies, specific to the infectious form of Toxoplasma gondii, would best meet the following specifications: anti-tachyzoïte neutralizing activity, reduction of side effects and lack of resistance to treatment. This strategy is innovative, as few or no similar studies have been published to date. Indeed, it is generally accepted by the scientific community that anti-toxoplasmic protective immune responses are mainly of a cellular nature, mediated by CD8+ T and CD4+ T lymphocytes, mainly via the secretion of interferon γ (IFN-γ), cytokine major resistance to infection. The humoral aspect of the immune response remains, to date, very little studied. However, the search for serum antibodies anti-Toxoplasma gondii represents the diagnostic and screening tool for toxoplasmosis and allows to define the serological status of uninfected individuals, in phase of seroconversion or chronically infected.

Antibodies, alone or in cooperation with cellular immunity, may be involved in limiting the spread of the parasite. The involvement of the antibodies in the anti-toxoplasmic defense has been demonstrated by the use of mice of phenotype μMT, deficient in B cells. These mice, after infection, develop a normal IFN-γ response but succumb to the infection in the cells. Four-week follow-up of cerebral parasite overload (Kang et al, Decreased resistance of B cell-deficient mice to infection with Toxoplasma gondii despite unimpaired expression of IFN-gamma. TNF-alpha, and inducible nitric oxide synthase, J Immunol., 2000 164, No. 5, pp 2629-34). Passive transfer of anti-T. gondii antibodies during infection of these same mice restores resistance to the parasite suggesting that the antibodies are capable of limiting infection (Johnson et al., Deficient humoral responses underlie susceptibility to Toxoplasma gondii in CD4-deficient mice, Infect. Immun, 2002, vol 70, No. 1, pp 185-91).

SAG1 protein is considered as the surface protein predominantly present on the tachyzoïte form of the parasite Toxoplasma gondii. SAG1 antigen is a protein of 336 amino acids, encoded by the sag1 gene. Toxoplasma gondii SAG1 protein is anchored in the cell membrane by glycosylphosphatidylinositol (GPI) anchorage (Wang et al., Research progress on surface antigen 1 (SAG1) of Toxoplasma gondii, Parasit Vectors, 2014, vol 7, p 180). The SAG1 protein of Toxoplasma gondii is also called P30. It consists of a D1 domain (N-terminal domain) of 129 amino acids and a D2 domain (C-terminal domain) of 126 amino acids (Graille et al., Crystal Structure of the Monomeric Form Toxoplasma gondii Surface Antigen 1 (SAG1) and a Monoclonal Antibody that Mimics the Human Immune Response, J. mol. BioL, 2005, 354, pp 447-458). It is implicated in the infectivity of the parasite Toxoplasma gondii, facilitating its approximation and attachment to the host cell (Grimwood and Smith, Toxoplasma gondii: the role of parasite surface and secreted proteins in host cell invasion, Int. J. Parasitol. 1996, vol 26, No. 2, pp 169-173; Robinson, Smith and Millner, Toxoplasma gondii major surface antigen (SAG1): In vitro analysis of host cell binding, Parasitology, 2004, Vol 128, No. 4, pp 391-396).

In the literature, Graille et al. (Graille et al., Crystal Structure of the Monomeric Complex of the Form of Toxoplasma gondii Surface Antigen 1 (SAG1) and a Monoclonal Antibody that Mimics the Human Immune Response. J. mol. Biol., 2005, 354, pp. 447-458) have described a monoclonal antibody (4F11E12) directed against the SAG1 protein capable of binding to the antigen at the D1 domain level with a nanomolar affinity, within a conformational epitope. However, in this study, the authors seem to indicate that this antibody would not be neutralizing insofar as it cannot prevent the homodimerization of the essential SAG1 protein to allow entry of the parasite into the host cell.

One of the aims of the invention is to provide peptide constructs, also referred to as “antibodies”, which inhibit the invasion of cells by the parasite Toxoplasma gondii.

Another aspect of the invention is to provide effective peptide constructs in the treatment of ocular toxoplasmosis, congenital toxoplasmosis and behavioral disorders related to the presence of Toxoplasma gondii.

One of the advantages of the invention is to provide peptide constructs capable of reducing or even preventing the appearance of ocular lesions due to Toxoplasma gondii.

Another advantage of the invention is to provide peptide constructs capable of reducing or preventing transplacental transmission of the parasite from mother to offspring.

The present invention relates to a peptide construct that does not contain a CH1 region, recognizing the SAG1 antigen of Toxoplasma gondii and capable of neutralizing the invasion of cells by Toxoplasma gondii, for its use in the treatment of toxoplasmosis.

The neutralization of the invasion of cells by Toxoplasma gondii can be demonstrated by the HFF neutralization test described in Section 5 (In vitro Neutralization Test) of Example 1: Materials and Methods. The optical density (OD) measured at 565 nm obtained with a peptide construct capable of neutralizing the invasion of the cells by Toxoplasma gondii will not be significantly different from the OD obtained with the HFF cells alone.

Surprisingly, the inventors have found that an antibody fragment directed against the SAG1 antigen of the parasite Toxoplasma gondii makes it possible to inhibit the invasion of the cells by the tachyzoïtes of Toxoplasma gondii and also that this fragment has a therapeutic effect on mouse models of ocular toxoplasmosis and congenital toxoplasmosis.

By “peptide construction” is meant any construction comprising a peptide part consisting of amino acids linked to one another by peptide bonds, in which said peptide part comprises at least one attachment domain to SAG1 antigen of Toxoplasma gondii. This term includes in particular the polypeptides alone and any physical association between two or more polypeptides, linked together by covalent bonds such as peptide bonds or disulfide bonds of cysteine residues, chemical or non-covalent bonds such as hydrogen bonds, van der Waals bonds, ionic and hydrophobic bonds. This term also includes the association of one or more polypeptides with one or more carbohydrate residues or chains.

For the purposes of the present invention, and by the term “peptide construct”, it is also understood to mean, whenever this term is used in the present description, an antibody, and in particular a monovalent antibody or a divalent antibody.

The peptide constructs according to the present invention are said to be glycosylated when they comprise at least one carbohydrate residue or at least one carbohydrate chain.

For the purposes of the present invention, the term “polypeptide” is intended to mean any polymer of amino acids linked together by peptide bonds, irrespective of its length. Thus, for the purposes of the present invention, the term polypeptide also encompasses peptides and proteins.

By “peptide construct or antibody recognizing the SAG1 antigen of Toxoplama gondii” is meant that said peptide construct or said antibody has at least one binding domain to the SAG1 antigen of Toxoplasma gondii.

By “binding domain” is meant any site capable of binding with affinity and specifically to another molecule (antigen).

By “any site capable of binding with affinity to an antigen” is meant by way of illustration any site for which the dissociation constant K_{d} characterizing the affinity of said antigen for said site is less than 10⁻⁷M.

The values of the association and dissociation constants characterizing the affinity of a peptide construct for an antigen can be measured by surface plasmon resonance (BIACORE), the antigen being immobilized on a Sensorchip. The use of surface plasmon resonance to measure the binding of a ligand to a receptor is a technique well known to those skilled in the art.

The terms SAG1 and P30 are used interchangeably in the present description and denote the same molecule.

The subject of the present invention is a peptide construct comprising the variable region of the heavy chain of a first antibody recognizing the SAG1 antigen of Toxoplasma gondii and which may contain all or part of the constant region devoid of CH1 region, of the heavy chain of a second antibody, said peptide construct recognizing the SAG1 antigen of Toxoplasma gondii and being capable of neutralizing the invasion of the cells by Toxoplasma gondii, for its use in the treatment of toxoplasmosis.

The invention relates to antibodies consisting solely of two heavy chains and antibodies consisting of two heavy chains and two light chains.

In the invention, the term “antibody” refers in particular to an immunoglobulin, a multimeric protein consisting of 4 chains participating in the acquired immune response.

Immunoglobulins are well known to those skilled in the art and consist of an assembly of two dimers each consisting of a heavy chain and a light chain. The multimeric complex assembled by the binding of a light chain and a heavy chain by a disulfide bridge between two cysteines, the two heavy chains being itself also interconnected by two disulfide bridges.

Each of the heavy chains and light chains consists of a constant region and a variable region. The assembly of the chains that make up an antibody makes it possible to define a characteristic three-dimensional structure in Y, where
the base of Y corresponds to the constant region Fc which is recognized by complement and Fc receptors, and
the ends of the arms of Y correspond to the respective assembly of the variable regions of the light chain and variables of the heavy chain.

More precisely, each light chain consists of a variable region (VL) and a constant region (CL). Each heavy chain consists of a variable region (VH) and a constant region consisting of three constant domains CH1, CH2 and CH3. The CH2 and CH3 domains make up the Fc domain.

The variable regions of the light chain and the heavy chain each consist of three antigen recognition regions (CDR) surrounded by four framework domains. The three-dimensional folding of the variable regions of the heavy chain and of the light chain is such that the 6 CDRs are exposed on the same side of the protein and allow the formation of a specific structure recognizing a determined antigen.

In the present description, the terms “variable domain” and “variable region” are used interchangeably. Similarly, the terms “constant domain” and “constant region” are used interchangeably.

According to a particular embodiment, the invention relates to a peptide construct comprising the variable region of the heavy chain of a first antibody recognizing the SAG1 antigen of Toxoplasma gondii and which may contain all or part of the constant region devoid of CH1 region of the heavy chain of a second antibody, all or part of the constant region devoid of CH1 region of the heavy chain of said second antibody making it possible to increase the half-life of said peptide construction under in vivo conditions, said peptide construct recognizing the SAG1 antigen of Toxoplasma gondii and being able to neutralize the invasion of the cells by Toxoplasma gondii, for its use in the treatment of toxoplasmosis.

The subject of the present invention is a peptide construct comprising the variable regions of the heavy chain and of the light chain of a first antibody recognizing the SAG1 antigen of Toxoplasma gondii and which may contain all or part of the constant region devoid of CH1 region of the heavy chain of a second antibody, said peptide construct recognizing the SAG1 antigen of Toxoplasma gondii and being capable of neutralizing the invasion of the cells by Toxoplasma gondii, for its use in the treatment of toxoplasmosis.

According to a particular embodiment, the invention relates to a peptide construct comprising the variable regions of the heavy chain and the light chain of a first antibody recognizing the SAG1 antigen of Toxoplasma gondii and which may contain all or part of the constant region lacking CH1 region, heavy chain of a second antibody,

said all or part of the constant region devoid of a CH1 region of the heavy chain of said second antibody making it possible to increase the half-life of said peptide construct under in vivo conditions,
said peptide construct recognizing the SAG1 antigen of Toxoplasma gondii and being able to neutralize the invasion of the cells by Toxoplasma gondii, for its use in the treatment of toxoplasmosis.

According to a particular embodiment, the invention relates to a peptide construct comprising the variable regions of the heavy chain and of the light chain of an antibody recognizing the SAG1 antigen of Toxoplasma gondii and which may contain all or part of the constant region devoid of CH1 region, of the heavy chain of the aforesaid antibody recognizing the SAG1 antigen of Toxoplasma gondii,

said peptide construct recognizing the SAG1 antigen of Toxoplasma gondii and being able to neutralize the invasion of the cells by Toxoplasma gondii, for its use in the treatment of toxoplasmosis.

According to a still more particular embodiment, the invention relates to a peptide construct as defined below, wherein said second antibody is a murine IgG2a immunoglobulin, for its use in the treatment of toxoplasmosis.

According to another particular embodiment, the invention relates to a peptide construct as defined below, wherein said first antibody recognizing the SAG1 antigen of Toxoplasma gondii is the monoclonal antibody 4F11E2, for its use in the treatment of toxoplasmosis.

The variable domain of the light chain of the monoclonal antibody 4F11E12 has a sequence encoded by the amino acid sequence SEQ ID NO: 1 and the variable domain of its heavy chain has a sequence encoded by the amino acid sequence SEQ ID NO: 2.

According to a particular embodiment, the invention relates to a peptide construct as defined above, for its use in the treatment of toxoplasmosis in humans.

According to a more particular embodiment, the invention relates to a peptide construct as defined above, for its use in the treatment of toxoplasmosis belonging to the group: ocular toxoplasmosis, congenital toxoplasmosis and behavioral disorders related to the presence of Toxoplasma gondii.

According to a particular embodiment, the invention relates to a peptide construct as defined above, recognizing the conformational epitope formed by the amino acids at positions 35 to 37, 39, 41, 42, 45, 48, 50, 59 at 65 and 112 to 114 of the amino acid sequence SEQ ID NO: 3, for its use in the treatment of toxoplasmosis.

The conformational epitope formed by the amino acids at positions 35 to 37, 39, 41, 42, 45, 48, 50, 59 to 65 and 112 to 114 of the amino acid sequence SEQ ID NO: 3 is the epitope recognized by the monoclonal antibody 4F11E12.

According to a particular embodiment, the invention relates to a peptide construct as defined above, comprising a CDR having at least 95%, at least 96%, at least 97%, at least 98% or at least 99% of identity with the sequence SEQ ID NO: 4, provided that said peptide construct retains its ability to neutralize the invasion of cells by Toxoplasma gondii, for its use in the treatment of toxoplasmosis.

According to a particular embodiment, the invention relates to a peptide construct as defined above, comprising a CDR having at least 95%, at least 96%, at least 97%, at least 98% or at least 99% of identity with the sequence SEQ ID NO: 5, provided that said peptide construct retains its ability to neutralize the invasion of cells by Toxoplasma gondii, for its use in the treatment of toxoplasmosis.

According to a particular embodiment, the invention relates to a peptide construct as defined above, comprising a CDR having at least 95%, at least 96%, at least 97%, at least 98% or at least 99% of identity with the sequence SEQ ID NO: 6, provided that said peptide construct retains its ability to neutralize the invasion of cells by Toxoplasma gondii, for its use in the treatment of toxoplasmosis.

According to a particular embodiment, the invention relates to a peptide construct as defined above, comprising a CDR having at least 95%, at least 96%, at least 97%, at least 98% or at least 99% of identity with the sequence SEQ ID NO: 7, provided that said peptide construct retains its ability to neutralize the invasion of the cells by Toxoplasma gondii, for its use in the treatment of toxoplasmosis.

According to a particular embodiment, the invention relates to a peptide construct as defined above, comprising a CDR having at least 95%, at least 96%, at least 97%, at least 98% or at least 99% of identity with the sequence SEQ ID NO: 8, provided that said peptide construct retains its ability to neutralize the invasion of the cells by Toxoplasma gondii, for its use in the treatment of toxoplasmosis.

According to a particular embodiment, the invention relates to a peptide construct as defined above, comprising a CDR having at least 95%, at least 96%, at least 97%, at least 98% or at least 99% of identity with the sequence SEQ ID NO: 9, provided that said peptide construct retains its ability to neutralize the invasion of cells by Toxoplasma gondii, for its use in the treatment of toxoplasmosis.

According to a more particular embodiment, the invention relates to a peptide construct as defined above, comprising the following six CDRs:

a CDR1 having at least 95%, at least 96%, at least 97%, at least 98% or at least 99% identity with the sequence SEQ ID NO: 4,
a CDR2 having at least 95%, at least 96%, at least 97%, at least 98% or at least 99% identity with the sequence SEQ ID NO: 5,
a CDR3 having at least 95%, at least 96%, at least 97%, at least 98% or at least 99% identity with the sequence SEQ ID NO: 6,
a CDR4 having at least 95%, at least 96%, at least 97%, at least 98% or at least 99% identity with the sequence SEQ ID NO: 7,
a CDR5 having at least 95%, at least 96%, at least 97%, at least 98% or at least 99% identity with the sequence SEQ ID NO: 8, and
a CDR6 having at least 95%, at least 96%, at least 97%, at least 98% or at least 99% identity with the sequence SEQ ID NO: 9,
provided that said peptide construct retains its ability to neutralize the invasion of cells by Toxoplasma gondii,
for its use in the treatment of toxoplasmosis.

According to a more particular embodiment, the invention relates to a peptide construct as defined above, comprising a CDR consisting of the amino acid sequence SEQ ID NO: 4, for its use in the treatment of toxoplasmosis.

According to a more particular embodiment, the invention relates to a peptide construct as defined above, comprising a CDR consisting of the amino acid sequence SEQ ID NO: 5, for its use in the treatment of toxoplasmosis.

According to a more particular embodiment, the invention relates to a peptide construct as defined above, comprising a CDR consisting of the amino acid sequence SEQ ID NO: 6, for its use in the treatment of toxoplasmosis.

According to a more particular embodiment, the invention relates to a peptide construct as defined above, comprising a CDR consisting of the amino acid sequence SEQ ID NO: 7, for its use in the treatment of toxoplasmosis.

According to a more particular embodiment, the invention relates to a peptide construct as defined above, comprising a CDR consisting of the amino acid sequence SEQ ID NO: 8, for its use in the treatment of toxoplasmosis.

According to a more particular embodiment, the invention relates to a peptide construct as defined above, comprising a CDR consisting of the amino acid sequence SEQ ID NO: 9, for its use in the treatment of toxoplasmosis.

According to a still more particular embodiment, the invention relates to a peptide construct as defined above, comprising the following six CDRs:

a CDR1 consisting of the amino acid sequence SEQ ID NO: 4,
a CDR2 consisting of the amino acid sequence SEQ ID NO: 5,
a CDR3 consisting of the amino acid sequence SEQ ID NO: 6,
a CDR4 consisting of the amino acid sequence SEQ ID NO: 7,
a CDR5 consisting of the amino acid sequence SEQ ID NO: 8 and
a CDR6 consisting of the amino acid sequence SEQ ID NO: 9,
for its use in the treatment of toxoplasmosis.

According to one particular embodiment, the invention relates to a peptide construct as defined above, devoid of the CH2 and CH3 regions of the aforementioned second antibody, for its use in the treatment of toxoplasmosis.

According to another particular embodiment, the invention relates to a peptide construct as defined above, comprising a CH3 region and devoid of CH2 region of the aforementioned second antibody, for its use in the treatment of toxoplasmosis.

According to another particular embodiment, the invention relates to a peptide construct as defined above, comprising a CH2 region and a CH3 region of the aforementioned second antibody, for its use in the treatment of toxoplasmosis.

According to another particular embodiment, the invention relates to a peptide construct as defined above, comprising a CH2 region and devoid of CH3 region of the aforementioned second antibody, for its use in the treatment of toxoplasmosis.

According to an advantageous embodiment, the invention relates to a peptide construct as defined above, chosen from: scFv, diabodies, single-chain diabodies, minibodies such as scFv-CH3 and diabody-CH3, scFv-Fc, diabody-Fc, for its use in the treatment of toxoplasmosis.

By “scFv” is meant a fusion protein consisting of the variable region of the heavy chain of an immunoglobulin and the variable region of the light chain of said immunoglobulin covalently bound to each other via a short peptide linker, comprising generally from 10 to 25 amino acids.

By “diabody” is meant a homodimer consisting of two peptide chains, themselves constituted by the variable region of the heavy chain of an immunoglobulin and the variable region of the light chain of said immunoglobulin covalently linked together via a peptide linker, generally comprising 5 amino acids and too short for the two variable regions to fold into scFv. The two subunits of diabody are bound together by weak bonds, such as hydrophobic bonds, hydrogen bonds, ionic bonds or van der Waals bonds.

By “single-chain diabody” is meant a fusion protein consisting of four variable domains derived from an immunoglobulin covalently linked together via three peptide linkers, said four variable domains consisting of two variable domains of the heavy chain and two variable domains of the light chain. The two variable domains arranged in the center of the molecule are a variable domain of the heavy chain and a variable domain of the light chain.

By “minibodies” is meant a protein consisting of a scFv fused with the CH3 domain of the immunoglobulin heavy chain or a homodimer consisting of a diabody fused to the CH3 domain of the immunoglobulin heavy chain. In the present description, the term “minibodies” refers to both scFv-CH3 and diabody-CH3.

By “scFv-CH3” is meant a protein consisting of a scFv fused with the CH3 domain of the heavy chain of an immunoglobulin.

By “diabody-CH3” is meant a homodimer consisting of a diabody in which each subunit is fused with the CH3 domain of the heavy chain of an immunoglobulin.

By “scFv-Fc” is meant a protein consisting of a scFv fused with the Fc fragment of an immunoglobulin, itself composed of the CH2 and CH3 domains of the heavy chain of said immunoglobulin. In the present description, scFv-Fc are also referred to as “scFv-CH2-CH3”.

By “diabody-Fc” is meant a homodimer consisting of a diabody in which each subunit is fused with the Fc domain of an immunoglobulin, itself composed of the CH2 and CH3 domains of the heavy chain of said immunoglobulin. In the present description, diabody-Fc are also referred to as “diabody-CH2-CH3”.

According to a particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 10 for its use in the treatment of toxoplasmosis.

The peptide construct of sequence SEQ ID NO: 10 is called SG0 in the present description.

According to a more particular embodiment, the invention relates to a peptide construct as defined above, comprising or consisting of a scFv consisting of the amino acid sequence SEQ ID NO: 10, for its use in the treatment of toxoplasmosis.

The amino acid sequence SEQ ID NO: 10 may be encoded by the nucleic acid sequence SEQ ID NO: 40.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 11, for its use in the treatment of toxoplasmosis.

The two subunits of diabody are bound together by weak bonds, such as hydrophobic bonds, hydrogen bonds, ionic bonds or van der Waals bonds.

The peptide construct of sequence SEQ ID NO: 11 is called SG5 in the present description.

According to a more particular embodiment, the invention relates to a peptide construct as defined above, comprising or consisting of a diabody consisting of two amino acid sequences of sequence SEQ ID NO: 11, for its use in the treatment of toxoplasmosis.

The amino acid sequence SEQ ID NO: 11 may be encoded by the nucleic acid sequence SEQ ID NO: 41.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, identity, at least 98% identity or at least 99% identity with the amino acid sequence SEQ ID NO: 12, for its use in the treatment of toxoplasmosis.

The peptide construct of sequence SEQ ID NO: 12 is called SG2-HL in the present description.

According to a more particular embodiment, the invention relates to a peptide construct as defined above, comprising or consisting of a scFv consisting of the amino acid sequence SEQ ID NO: 12, for its use in the treatment of toxoplasmosis.

The amino acid sequence SEQ ID NO: 12 may be encoded by the nucleic acid sequence SEQ ID NO: 42.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 13, for its use in the treatment of toxoplasmosis.

The peptide construct of sequence SEQ ID NO: 13 is called DbSG2 in the present description.

According to a more particular embodiment, the invention relates to a peptide construct as defined above, comprising or consisting of a diabody consisting of two amino acid sequences of sequence SEQ ID NO: 13, for its use in the treatment of toxoplasmosis. The amino acid sequence SEQ ID NO: 13 can be encoded by the nucleic acid sequence SEQ ID NO: 43.

According to another particular embodiment, the invention relates to a peptide construct as defined above, comprising or consisting of a scFv-CH3 consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 14, for its use in the treatment of toxoplasmosis.

The peptide construct of sequence SEQ ID NO: 14 is called SG2-CH3 in the present description.

According to a more particular embodiment, the invention relates to a peptide construct as defined above, comprising or consisting of a scFv-CH3 consisting of the amino acid sequence SEQ ID NO: 14, for its use in the treatment of toxoplasmosis.

The amino acid sequence SEQ ID NO: 14 can be encoded by the nucleic acid sequence SEQ ID NO: 44.

According to another particular embodiment, the invention relates to a peptide construct as defined above, comprising or consisting of a diabody-CH3 consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 15, for its use in the treatment of toxoplasmosis.

The two subunits of diabody-CH3 are bound together by weak bonds, such as hydrophobic bonds, hydrogen bonds, ionic bonds or van der Waals bonds.

The peptide construct of sequence SEQ ID NO: 15 is called DbSG2-CH3 in the present description.

According to a more particular embodiment, the invention relates to a peptide construct as defined above, comprising or consisting of a diabody-CH3 consisting of two amino acid sequences of sequence SEQ ID NO: 15, for its use in the treatment of toxoplasmosis.

The amino acid sequence SEQ ID NO: 15 may be encoded by the nucleic acid sequence SEQ ID NO: 45.

According to another particular embodiment, the invention relates to a peptide construct as defined above, comprising or consisting of a scFv-Fc consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 16, for its use in the treatment of toxoplasmosis.

The peptide construct of sequence SEQ ID NO: 16 is called SG2-Fc2a in the present description.

According to a more particular embodiment, the invention relates to a peptide construct as defined above, comprising or consisting of a scFv-Fc consisting of the amino acid sequence SEQ ID NO: 16, for its use in the treatment of toxoplasmosis.

The amino acid sequence SEQ ID NO: 16 may be encoded by the nucleic acid sequence SEQ ID NO: 46.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 17, for its use in the treatment of toxoplasmosis.

The peptide construct of sequence SEQ ID NO: 17 is called scFvSG1 in the present description.

According to a more particular embodiment, the invention relates to a peptide construct as defined above, comprising or consisting of a scFv consisting of the amino acid sequence SEQ ID NO: 17, for its use in the treatment of toxoplasmosis.

The amino acid sequence SEQ ID NO: 17 can be encoded by the nucleic acid sequence SEQ ID NO: 47.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 18, for its use in the treatment of toxoplasmosis.

The peptide construct of sequence SEQ ID NO: 18 is called DbF3S2 in the present description.

According to a more particular embodiment, the invention relates to a peptide construct as defined above, comprising or consisting of a diabody consisting of two amino acid sequences of sequence SEQ ID NO: 18, for its use in the treatment of toxoplasmosis.

The amino acid sequence SEQ ID NO: 18 can be encoded by the nucleic acid sequence SEQ ID NO: 48.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 19, for its use in the treatment of toxoplasmosis.

The peptide construct of sequence SEQ ID NO: 19 is called DbF4S2 in the present description.

According to a more particular embodiment, the invention relates to a peptide construct as defined above, comprising or consisting of a diabody consisting of two amino acid sequences of sequence SEQ ID NO: 19, for its use in the treatment of toxoplasmosis. The amino acid sequence SEQ ID NO: 19 can be encoded by the nucleic acid sequence SEQ ID NO: 49.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 20, for its use in the treatment of toxoplasmosis.

The peptide construct of sequence SEQ ID NO: 20 is called scFvF5S2 in the present description.

According to a more particular embodiment, the invention relates to a peptide construct as defined above, comprising or consisting of a scFv consisting of the amino acid sequence SEQ ID NO: 20, for its use in the treatment of toxoplasmosis.

The amino acid sequence SEQ ID NO: 20 may be encoded by the nucleic acid sequence SEQ ID NO: 50.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 21, for its use in the treatment of toxoplasmosis.

The peptide construct of sequence SEQ ID NO: 21 is called scFvF5S4 in the present description.

According to a more particular embodiment, the invention relates to a peptide construct as defined above, comprising or consisting of a scFv consisting of the amino acid sequence SEQ ID NO: 21, for its use in the treatment of toxoplasmosis.

The amino acid sequence SEQ ID NO: 21 can be encoded by the nucleic acid sequence SEQ ID NO: 51. According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 22, for its use in the treatment of toxoplasmosis.

The peptide construct of sequence SEQ ID NO: 22 is called scFvF5S5 in the present description.

According to a more particular embodiment, the invention relates to a peptide construct as defined above, comprising or consisting of a scFv consisting of the amino acid sequence SEQ ID NO: 22, for its use in the treatment of toxoplasmosis.

The amino acid sequence SEQ ID NO: 22 may be encoded by the nucleic acid sequence SEQ ID NO: 52.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 23, for its use in the treatment of toxoplasmosis.

The peptide construct of sequence SEQ ID NO: 23 is called scFvF5S6 in the present description.

According to a more particular embodiment, the invention relates to a peptide construct as defined above, comprising or consisting of a scFv consisting of the amino acid sequence SEQ ID NO: 23, for its use in the treatment of toxoplasmosis.

The amino acid sequence SEQ ID NO: 23 can be encoded by the nucleic acid sequence SEQ ID NO: 53.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 24, for its use in the treatment of toxoplasmosis.

The peptide construct of sequence SEQ ID NO: 24 is called Hum-scFvH2S in the present description.

According to a more particular embodiment, the invention relates to a peptide construct as defined above, comprising or consisting of a scFv consisting of the amino acid sequence SEQ ID NO: 24, for its use in the treatment of toxoplasmosis.

The amino acid sequence SEQ ID NO: 24 can be encoded by the nucleic acid sequence SEQ ID NO: 54.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 25, for its use in the treatment of toxoplasmosis.

The peptide construct of sequence SEQ ID NO: 25 is called SG0-LH in the present description.

According to a more particular embodiment, the invention relates to a peptide construct as defined above, comprising or consisting of a scFv consisting of the amino acid sequence SEQ ID NO: 25, for its use in the treatment of toxoplasmosis.

The amino acid sequence SEQ ID NO: 25 can be encoded by the nucleic acid sequence SEQ ID NO: 55.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 26, for its use in the treatment of toxoplasmosis.

The peptide construct of sequence SEQ ID NO: 26 is called SG5-LH in the present description.

According to a more particular embodiment, the invention relates to a peptide construct as defined above, comprising or consisting of a diabody consisting of two amino acid sequences of sequence SEQ ID NO: 26, for its use in the treatment of toxoplasmosis.

The amino acid sequence SEQ ID NO: 26 can be encoded by the nucleic acid sequence SEQ ID NO: 56.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 27, for its use in the treatment of toxoplasmosis.

The peptide construct of sequence SEQ ID NO: 27 is called SG2-LH in the present description.

According to a more particular embodiment, the invention relates to a peptide construct as defined above, comprising or consisting of a scFv consisting of the amino acid sequence SEQ ID NO: 27, for its use in the treatment of toxoplasmosis.

The amino acid sequence SEQ ID NO: 27 can be encoded by the nucleic acid sequence SEQ ID NO: 57.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 28, for its use in the treatment of toxoplasmosis.

The peptide construct of sequence SEQ ID NO: 28 is called DbSG2-LH in the present description.

According to a more particular embodiment, the invention relates to a peptide construct as defined above, comprising or consisting of a diabody consisting of two amino acid sequences of sequence SEQ ID NO: 28, for its use in the treatment of toxoplasmosis.

The amino acid sequence SEQ ID NO: 28 may be encoded by the nucleic acid sequence SEQ ID NO: 58.

According to another particular embodiment, the invention relates to a peptide construct as defined above, comprising or consisting of a scFv-CH3 consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 29 for its use in the treatment of toxoplasmosis.

The peptide construct of sequence SEQ ID NO: 29 is called SG2-CH3-LH in the present description.

According to a more particular embodiment, the invention relates to a peptide construct as defined above, comprising or consisting of a scFv-CH3 consisting of the amino acid sequence SEQ ID NO: 29, for its use in the treatment of toxoplasmosis.

The amino acid sequence SEQ ID NO: 29 may be encoded by the nucleic acid sequence SEQ ID NO: 59.

According to another particular embodiment, the invention relates to a peptide construct as defined above, comprising or consisting of a diabody-CH3 consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 30 for its use in the treatment of toxoplasmosis. The peptide construct of sequence SEQ ID NO: 30 is called DbSG2-CH3-LH in the present description.

According to a more particular embodiment, the invention relates to a peptide construct as defined above, comprising or consisting of a diabody-CH3 consisting of two amino acid sequences of sequence SEQ ID NO: 30, for its use in the treatment of toxoplasmosis.

The amino acid sequence SEQ ID NO: 30 can be encoded by the nucleic acid sequence SEQ ID NO: 60.

According to another particular embodiment, the invention relates to a peptide construct as defined above, comprising or consisting of a scFv-Fc consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 31, for its use in the treatment of toxoplasmosis.

The peptide construct of sequence SEQ ID NO: 31 is called SG2-Fc2a-LH in the present description.

According to a more particular embodiment, the invention relates to a peptide construct as defined above, comprising or consisting of a scFv-Fc consisting of the amino acid sequence SEQ ID NO: 31, for its use in the treatment of toxoplasmosis.

The amino acid sequence SEQ ID NO: 31 may be encoded by the nucleic acid sequence SEQ ID NO: 61.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 32 for its use in the treatment of toxoplasmosis.

The peptide construct of sequence SEQ ID NO: 32 is called scFvSG1-LH in the present description.

According to a more particular embodiment, the invention relates to a peptide construct as defined above, comprising or consisting of a scFv consisting of the amino acid sequence SEQ ID NO: 32, for its use in the treatment of toxoplasmosis.

The amino acid sequence SEQ ID NO: 32 can be encoded by the nucleic acid sequence SEQ ID NO: 62.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 33, for its use in the treatment of toxoplasmosis.

The peptide construct of sequence SEQ ID NO: 33 is called DbF3S2-LH in the present description.

According to a more particular embodiment, the invention relates to a peptide construct as defined above, comprising or consisting of a diabody consisting of two amino acid sequences of sequence SEQ ID NO: 33, for its use in the treatment of toxoplasmosis.

The amino acid sequence SEQ ID NO: 33 can be encoded by the nucleic acid sequence SEQ ID NO: 63.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 34, for use in the treatment of toxoplasmosis.

The peptide construct of sequence SEQ ID NO: 34 is called DbF4S2-LH in the present description.

According to a more particular embodiment, the invention relates to a peptide construct as defined above, comprising or consisting of a diabody consisting of two amino acid sequences of sequence SEQ ID NO: 34, for its use in the treatment of toxoplasmosis.

The amino acid sequence SEQ ID NO: 34 can be encoded by the nucleic acid sequence SEQ ID NO: 64.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 35, for its use in the treatment of toxoplasmosis.

The peptide construct of sequence SEQ ID NO: 35 is called scFvF5S2-LH in the present description.

According to a more particular embodiment, the invention relates to a peptide construct as defined above, comprising or consisting of a scFv consisting of the amino acid sequence SEQ ID NO: 35, for its use in the treatment of toxoplasmosis.

The amino acid sequence SEQ ID NO: 35 can be encoded by the nucleic acid sequence SEQ ID NO: 65.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 36, for its use in the treatment of toxoplasmosis.

The peptide construct of sequence SEQ ID NO: 36 is called scFvF5S4-LH in the present description.

According to a more particular embodiment, the invention relates to a peptide construct as defined above, comprising or consisting of a scFv consisting of the amino acid sequence SEQ ID NO: 36, for its use in the treatment of toxoplasmosis. The amino acid sequence SEQ ID NO: 36 can be encoded by the nucleic acid sequence SEQ ID NO: 66.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 37, for its use in the treatment of toxoplasmosis.

The peptide construct of sequence SEQ ID NO: 37 is called scFvF5S5-LH in the present description.

According to a more particular embodiment, the invention relates to a peptide construct as defined above, comprising or consisting of a scFv consisting of the amino acid sequence SEQ ID NO: 37, for its use in the treatment of toxoplasmosis.

The amino acid sequence SEQ ID NO: 37 may be encoded by the nucleic acid sequence SEQ ID NO: 67.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 38 for its use in the treatment of toxoplasmosis.

The peptide construct of sequence SEQ ID NO: 38 is called scFvF5S6-LH in the present description.

According to a more particular embodiment, the invention relates to a peptide construct as defined above, comprising or consisting of a scFv consisting of the amino acid sequence SEQ ID NO: 38, for its use in the treatment of toxoplasmosis.

The amino acid sequence SEQ ID NO: 38 can be encoded by the nucleic acid sequence SEQ ID NO: 68. According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 39, for its use in the treatment of toxoplasmosis.

The peptide construct of sequence SEQ ID NO: 39 is called Hum-scFvH2S-LH in the present description.

According to a more particular embodiment, the invention relates to a peptide construct as defined above, comprising or consisting of a scFv consisting of the amino acid sequence SEQ ID NO: 39, for its use in the treatment of toxoplasmosis.

The amino acid sequence SEQ ID NO: 39 can be encoded by the nucleic acid sequence SEQ ID NO: 69.

The invention also relates to a peptide construct comprising the heavy chain variable region (VHH) of a first antibody recognizing the SAG1 antigen of Toxoplasma gondii and which may contain all or part of the constant region devoid of CH1 region, of the heavy chain with a second antibody, said peptide construct recognizing the SAG1 antigen of Toxoplasma gondii and being able to neutralize the invasion of the cells by Toxoplasma gondii, for its use in the treatment of toxoplasmosis.

Said peptide construction is then devoid of variable light chain region. In this embodiment, said first antibody recognizing the SAG1 antigen of Toxoplasma gondii is a camelid antibody consisting of two heavy chains and lacking a light chain. The heavy chains of these antibodies comprise a variable domain (VHH) and a constant domain.

According to another particular embodiment, the invention relates to a peptide construct as defined above, for its use as a drug.

According to a more particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 10, for use as a drug.

According to a more particular embodiment, the invention relates to a peptide construct as defined above, comprising or consisting of a scFv consisting of the amino acid sequence SEQ ID NO: 10, for its use as a drug.

According to another aspect, the invention relates to a pharmaceutical composition comprising, as active substance, a peptide construct containing no CH1 region, recognizing the SAG1 antigen of Toxoplasma gondii and capable of neutralizing the invasion of the cells by Toxoplasma gondii, optionally in combination with a pharmaceutically acceptable carrier.

According to a particular embodiment, the invention relates to a pharmaceutical composition comprising as active substance a peptide construct comprising the variable region of the heavy chain of a first antibody recognizing the SAG1 antigen of Toxoplasma gondii and which may contain all or part of the constant region devoid of CH1 region, of the heavy chain of a second antibody, said peptide construct recognizing the SAG1 antigen of Toxoplasma gondii and being capable of neutralizing the invasion of the cells by Toxoplasma gondii,

optionally in combination with a pharmaceutically acceptable carrier.

According to a particular embodiment, the invention relates to a pharmaceutical composition comprising as active substance a peptide construct comprising the variable region of the heavy chain of a first antibody recognizing the SAG1 antigen of Toxoplasma gondii and which may contain all or part of the constant region devoid of CH1 region, of the heavy chain of a second antibody, all or part of the constant region devoid of CH1 region of the heavy chain of said second antibody to increase the half-life of said construction peptide under in vivo conditions, said peptide construct recognizing the SAG1 antigen of Toxoplasma gondii and being capable of neutralizing the invasion of the cells by Toxoplasma gondii, optionally in association with a pharmaceutically acceptable vehicle.

According to one particular embodiment, the invention relates to a pharmaceutical composition comprising, as active substance, a peptide construct comprising the variable regions of the heavy chain and of the light chain of a first antibody recognizing the SAG1 antigen of Toxoplasma gondii and which may contain all or part of the CH1 region-free constant region of the heavy chain of a second antibody, said peptide construct recognizing the SAG1 antigen of Toxoplasma gondii and being able to neutralize the invasion of the cells by Toxoplasma gondii optionally in combination with a pharmaceutically acceptable carrier.

According to one particular embodiment, the invention relates to a pharmaceutical composition comprising, as active substance, a peptide construct comprising the variable regions of the heavy chain and of the light chain of a first antibody recognizing the SAG1 antigen of Toxoplasma gondii and may contain all or part of the constant region devoid of CH1 region, of the heavy chain of a second antibody, said all or part of the constant region devoid of CH1 region of the heavy chain of said second antibody making it possible to increase the half-life of said peptide construct under in vivo conditions, said peptide construct recognizing the SAG antigen of Toxoplasma gondii and being able to neutralize the invasion of the cells by Toxoplasma gondii, optionally in combination with a pharmaceutically acceptable carrier.

According to a more particular embodiment, the invention relates to a pharmaceutical composition as defined above, wherein said second antibody is a murine IgG2a immunoglobulin.

According to a more particular embodiment, the invention relates to a pharmaceutical composition as defined above, wherein said first antibody recognizing the SAG1 antigen of Toxoplasma gondii is the monoclonal antibody 4F11E12.

According to a more particular embodiment, the invention relates to a pharmaceutical composition as defined above, wherein said peptide construct recognizes the conformational epitope formed by the amino acids at positions 35 to 37, 39, 41, 42, 45, 48, 50, 59 to 65 and 112 to 114 of the amino acid sequence SEQ ID NO: 3.

According to a particular embodiment, the invention relates to a pharmaceutical composition as defined above, wherein said peptide construct comprises a CDR having at least 95%, at least 96%, at least 97%, at least 98% or at least 99% identity with the sequence SEQ ID NO: 4, provided that said peptide construct retains its ability to neutralize the invasion of the cells by Toxoplasma gondii.

According to a particular embodiment, the invention relates to a pharmaceutical composition as defined above, wherein said peptide construct comprises a CDR having at least 95%, at least 96%, at least 97%, at least 98% or at least 99% identity with the sequence SEQ ID NO: 5, provided that said peptide construct retains its ability to neutralize the invasion of the cells by Toxoplasma gondii.

According to a particular embodiment, the invention relates to a pharmaceutical composition as defined above, wherein said peptide construct comprises a CDR having at least 95%, at least 96%, at least 97%, at least 98% or at least 99% identity with the sequence SEQ ID NO: 6, provided that said peptide construct retains its ability to neutralize the invasion of the cells by Toxoplasma gondii.

According to a particular embodiment, the invention relates to a pharmaceutical composition as defined above, wherein said peptide construct comprises a CDR having at least 95%, at least 96%, at least 97%, at least 98% or at least 99% identity with the sequence SEQ ID NO: 7, provided that said peptide construct retains its ability to neutralize the invasion of the cells by Toxoplasma gondii.

According to a particular embodiment, the invention relates to a pharmaceutical composition as defined above, wherein said peptide construct comprises a CDR having at least 95%, at least 96%, at least 97%, at least 98% or at least 99% identity with the sequence SEQ ID NO: 8 provided that said peptide construct retains its ability to neutralize the invasion of the cells by Toxoplasma gondii.

According to a particular embodiment, the invention relates to a pharmaceutical composition as defined above, wherein said peptide construct comprises a CDR having at least 95%, at least 96%, at least 97%, at least 98% or at least 99% identity with the sequence SEQ ID NO: 9, provided that said peptide construct retains its ability to neutralize the invasion of the cells by Toxoplasma gondii.

According to a more particular embodiment, the invention relates to a pharmaceutical composition as defined above, wherein said peptide construct comprises the following six CDRs:

a CDR1 having at least 95%, at least 96%, at least 97%, at least 98% or at least 99% identity with the sequence SEQ ID NO: 4,
a CDR2 having at least 95%, at least 96%, at least 97%, at least 98% or at least 99% identity with the sequence SEQ ID NO: 5,
a CDR3 having at least 95%, at least 96%, at least 97%, at least 98% or at least 99% identity with the sequence SEQ ID NO: 6,
a CDR4 having at least 95%, at least 96%, at least 97%, at least 98% or at least 99% identity with the sequence SEQ ID NO: 7,
a CDR5 having at least 95%, at least 96%, at least 97%, at least 98% or at least 99% identity with the sequence SEQ ID NO: 8, and
a CDR6 having at least 95%, at least 96%, at least 97%, at least 98% or at least 99% identity with the sequence SEQ ID NO: 9, provided that said peptide construct retains its ability to neutralize cell invasion by Toxoplasma gondii.

According to a more particular embodiment, the invention relates to a pharmaceutical composition as defined above, wherein said peptide construct comprises a CDR consisting of the amino acid sequence SEQ ID NO: 4.

According to a more particular embodiment, the invention relates to a pharmaceutical composition as defined above, wherein said peptide construct comprises a CDR consisting of the amino acid sequence SEQ ID NO: 5.

According to a more particular embodiment, the invention relates to a pharmaceutical composition as defined above, wherein said peptide construct comprises a CDR consisting of the amino acid sequence SEQ ID NO: 6.

According to a more particular embodiment, the invention relates to a pharmaceutical composition as defined above, wherein said peptide construct comprises a CDR consisting of the amino acid sequence SEQ ID NO: 7. According to a more particular embodiment, the invention relates to a pharmaceutical composition as defined above, wherein said peptide construct comprises a CDR consisting of the amino acid sequence SEQ ID NO: 8.

According to a more particular embodiment, the invention relates to a pharmaceutical composition as defined above, wherein said peptide construct comprises a CDR consisting of the amino acid sequence SEQ ID NO: 9.

According to a more particular embodiment, the invention relates to a pharmaceutical composition as defined above, wherein said peptide construct comprises the following six CDRs:

a CDR1 consisting of the amino acid sequence SEQ ID NO: 4,
a CDR2 consisting of the amino acid sequence SEQ ID NO: 5,
a CDR3 consisting of the amino acid sequence SEQ ID NO: 6,
a CDR4 consisting of the amino acid sequence SEQ ID NO: 7,
a CDR5 consisting of the amino acid sequence SEQ ID NO: 8 and
a CDR6 consisting of the amino acid sequence SEQ ID NO: 9.

According to a particular embodiment, the invention relates to a pharmaceutical composition as defined above, wherein said peptide construct is devoid of the CH2 and CH3 regions of the aforesaid second antibody.

According to another particular embodiment, the invention relates to a pharmaceutical composition as defined above, wherein said peptide construct comprises a CH3 region and is devoid of CH2 region of said second antibody.

According to another particular embodiment, the invention relates to a pharmaceutical composition as defined above, wherein said peptide construct comprises a CH2 region and a CH3 region of the aforesaid second antibody.

According to another particular embodiment, the invention relates to a pharmaceutical composition as defined above, wherein said peptide construct comprises a CH2 region and is devoid of CH3 region of said second antibody.

According to one particular embodiment, the invention relates to a pharmaceutical composition as defined above, wherein said peptide construction is chosen from: scFv, diabodies, single-chain diabodies, minibodies such as scFv-CH3 and diabodies-CH3, scFv-Fc, diabody-Fc.

According to a particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 10.

According to another more particular embodiment, the invention relates to a pharmaceutical composition as defined above, wherein said peptide construct comprises or consists of a scFv consisting of the amino acid sequence SEQ ID NO: 10.

According to another particular embodiment, the invention relates to a pharmaceutical composition as defined above, wherein said peptide construct comprises or consists of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 11.

The two subunits of diabody are bound together by weak bonds, such as hydrophobic bonds, hydrogen bonds, ionic bonds or van der Waals bonds.

According to another more particular embodiment, the invention relates to a pharmaceutical composition as defined above, wherein said peptide construct comprises or consists of a diabody consisting of two amino acid sequences of sequence SEQ ID NO: 11.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 12.

According to a more particular embodiment, the invention relates to a pharmaceutical composition as defined above, wherein said peptide construct comprises or consists of a scFv consisting of the amino acid sequence SEQ ID NO: 12.

According to another particular embodiment, the invention relates to a pharmaceutical composition as defined above, wherein said peptide construct comprises or consists of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 13.

According to another more particular embodiment, the invention relates to a pharmaceutical composition as defined above, wherein said peptide construct comprises or consists of a diabody consisting of two amino acid sequences of sequence SEQ ID NO: 13.

According to another particular embodiment, the invention relates to a pharmaceutical composition as defined above, wherein said peptide construct comprises or consists of a scFv-CH3 consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 14.

According to another more particular embodiment, the invention relates to a pharmaceutical composition as defined above, wherein said peptide construct comprises or consists of a scFv-CH3 consisting of the amino acid sequence SEQ ID NO: 14.

According to another particular embodiment, the invention relates to a pharmaceutical composition as defined above, wherein said peptide construct comprises or consists of a diabody-CH3 consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 15.

The two subunits of diabody-CH3 are bound together by weak bonds, such as hydrophobic bonds, hydrogen bonds, ionic bonds or van der Waals bonds.

According to another more particular embodiment, the invention relates to a pharmaceutical composition as defined above, wherein said peptide construct comprises or consists of a diabody-CH3 consisting of two amino acid sequences of sequence SEQ ID NO: 15.

According to another particular embodiment, the invention relates to a pharmaceutical composition as defined above, wherein said peptide construct comprises or consists of a scFv-Fc consisting of an amino acid sequence having at least 90% identity, at least 91% identity, at least 92% identity, at least 93% identity, at least 94% identity, at least 95% identity, at least 96% identity at least 97% identity, at least 98% identity or at least 99% identity with the amino acid sequence SEQ ID NO: 16.

According to another more particular embodiment, the invention relates to a pharmaceutical composition as defined above, wherein said peptide construct comprises or consists of a scFv-Fc consisting of the amino acid sequence SEQ ID NO: 16.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 17.

According to another more particular embodiment, the invention relates to a pharmaceutical composition as defined above, wherein said peptide construct comprises or consists of a scFv consisting of the amino acid sequence SEQ ID NO: 17.

According to another particular embodiment, the invention relates to a pharmaceutical composition as defined above, wherein said peptide construct comprises or consists of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 18.

According to another more particular embodiment, the invention relates to a pharmaceutical composition as defined above, wherein said peptide construct comprises or consists of a diabody consisting of two amino acid sequences of sequence SEQ ID NO: 18.

According to another particular embodiment, the invention relates to a pharmaceutical composition as defined above, wherein said peptide construct comprises or consists of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 19.

According to another more particular embodiment, the invention relates to a pharmaceutical composition as defined above, wherein said peptide construct comprises or consists of a diabody consisting of two amino acid sequences of sequence SEQ ID NO: 19.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 20.

According to another more particular embodiment, the invention relates to a pharmaceutical composition as defined above, wherein said peptide construct comprises or consists of a scFv consisting of the amino acid sequence SEQ ID NO: 20.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 21.

According to another more particular embodiment, the invention relates to a pharmaceutical composition as defined above, wherein said peptide construct comprises or consists of a scFv consisting of the amino acid sequence SEQ ID NO: 21.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 22.

According to another more particular embodiment, the invention relates to a pharmaceutical composition as defined above, wherein said peptide construct comprises or consists of a scFv consisting of the amino acid sequence SEQ ID NO: 22.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 23.

According to another more particular embodiment, the invention relates to a pharmaceutical composition as defined above, wherein said peptide construct comprises or consists of a scFv consisting of the amino acid sequence SEQ ID NO: 23.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 24.

According to another more particular embodiment, the invention relates to a pharmaceutical composition as defined above, wherein said peptide construct comprises or consists of a scFv consisting of the amino acid sequence SEQ ID NO: 24.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 25.

According to another more particular embodiment, the invention relates to a pharmaceutical composition as defined above, wherein said peptide construct comprises or consists of a scFv consisting of the amino acid sequence SEQ ID NO: 25.

According to another particular embodiment, the invention relates to a pharmaceutical composition as defined above, wherein said peptide construct comprises or consists of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 26.

According to another more particular embodiment, the invention relates to a pharmaceutical composition as defined above, wherein said peptide construct comprises or consists of a diabody consisting of two amino acid sequences of sequence SEQ ID NO: 26.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 27.

According to another more particular embodiment, the invention relates to a pharmaceutical composition as defined above, wherein said peptide construct comprises or consists of a scFv consisting of the amino acid sequence SEQ ID NO: 27.

According to another particular embodiment, the invention relates to a pharmaceutical composition as defined above, wherein said peptide construct comprises or consists of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 28.

According to another more particular embodiment, the invention relates to a pharmaceutical composition as defined above, wherein said peptide construct comprises or consists of a diabody consisting of two amino acid sequences of sequence SEQ ID NO: 28.

According to another particular embodiment, the invention relates to a pharmaceutical composition as defined above, wherein said peptide construct comprises or consists of a scFv-CH3 consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 29.

According to another more particular embodiment, the invention relates to a pharmaceutical composition as defined above, wherein said peptide construct comprises or consists of a scFv-CH3 consisting of the amino acid sequence SEQ ID NO: 29.

According to another particular embodiment, the invention relates to a pharmaceutical composition as defined above, wherein said peptide construct comprises or consists of a diabody-CH3 consisting of two identical amino acid sequences having at least 90% identity, at least 91% identity, at least 92% identity, at least 93% identity, at least 94% identity, at least 95% identity, at least 96% identity at least 97% identity, at least 98% identity or at least 99% identity with the amino acid sequence SEQ ID NO: 30.

According to another more particular embodiment, the invention relates to a pharmaceutical composition as defined above, wherein said peptide construct comprises or consists of a diabody-CH3 consisting of two amino acid sequences of sequence SEQ ID NO: 30.

According to another particular embodiment, the invention relates to a pharmaceutical composition as defined above, wherein said peptide construct comprises or consists of a scFv-Fc consisting of an amino acid sequence having at least 90% identity, at least 91% identity, at least 92% identity, at least 93% identity, at least 94% identity, at least 95% identity, at least 96% identity at least 97% identity, at least 98% identity or at least 99% identity with the amino acid sequence SEQ ID NO: 31.

According to another more particular embodiment, the invention relates to a pharmaceutical composition as defined above, wherein said peptide construct comprises or consists of a scFv-Fc consisting of the amino acid sequence SEQ ID NO: 31.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 32.

According to another more particular embodiment, the invention relates to a pharmaceutical composition as defined above, wherein said peptide construct comprises or consists of a scFv consisting of the amino acid sequence SEQ ID NO: 32.

According to another particular embodiment, the invention relates to a pharmaceutical composition as defined above, wherein said peptide construct comprises or consists of a diabody consisting of two identical amino acid sequences having at least 90% of identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 33.

According to another more particular embodiment, the invention relates to a pharmaceutical composition as defined above, wherein said peptide construct comprises or consists of a diabody consisting of two amino acid sequences of sequence SEQ ID NO: 33.

According to another particular embodiment, the invention relates to a pharmaceutical composition as defined above, wherein said peptide construct comprises or consists of a diabody consisting of two identical amino acid sequences having at least 90% identity, at least 91% identity, at least 92% identity, at least 93% identity, at least 94% identity, at least 95% identity, at least 96% identity, at least at least 97% identity, at least 98% identity or at least 99% identity with the amino acid sequence SEQ ID NO: 34.

According to another more particular embodiment, the invention relates to a pharmaceutical composition as defined above, wherein said peptide construct comprises or consists of a diabody consisting of two amino acid sequences of sequence SEQ ID NO: 34.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least at least 97% identity, at least 98% identity or at least 99% identity with the amino acid sequence SEQ ID NO: 35.

According to another more particular embodiment, the invention relates to a pharmaceutical composition as defined above, wherein said peptide construct comprises or consists of a scFv consisting of the amino acid sequence SEQ ID NO: 35.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least at least 97% identity, at least 98% identity or at least 99% identity with the amino acid sequence SEQ ID NO: 36.

According to another more particular embodiment, the invention relates to a pharmaceutical composition as defined above, wherein said peptide construct comprises or consists of a scFv consisting of the amino acid sequence SEQ ID NO: 36.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least at least 97% identity, at least 98% identity or at least 99% identity with the amino acid sequence SEQ ID NO: 37.

According to another more particular embodiment, the invention relates to a pharmaceutical composition as defined above, wherein said peptide construct comprises or consists of a scFv consisting of the amino acid sequence SEQ ID NO: 37.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least at least 97% identity, at least 98% identity or at least 99% identity with the amino acid sequence SEQ ID NO: 38.

According to another more particular embodiment, the invention relates to a pharmaceutical composition as defined above, wherein said peptide construct comprises or consists of a scFv consisting of the amino acid sequence SEQ ID NO: 38.

According to another particular embodiment, the invention relates to a pharmaceutical composition as defined above, wherein said peptide construct comprises or consists of a scFv consisting of an amino acid sequence having at least 90% identity, at least 91% identity, at least 92% identity, at least 93% identity, at least 94% identity, at least 95% identity, at least 96% identity, at least at least 97% identity, at least 98% identity or at least 99% identity with the amino acid sequence SEQ ID NO: 39.

According to another more particular embodiment, the invention relates to a pharmaceutical composition as defined above, wherein said peptide construct comprises or consists of a scFv consisting of the amino acid sequence SEQ ID NO: 39.

According to a particular embodiment, the invention relates to a pharmaceutical composition as defined above, wherein said peptide construct is administrable at a dose of 5 μg/kg to 50 mg/kg. According to a more particular embodiment, the invention relates to a pharmaceutical composition as defined above, wherein said peptide construction is administrable at a dose of 5 to 100 μg/kg, 100 to 500 μg/kg, 500 μg/kg at 1 mg/kg, 1 to 15 mg/kg, 15 to 30 mg/kg or 30 to 50 mg/kg.

According to a particular embodiment, the invention relates to a pharmaceutical composition as defined above, wherein said peptide construct is in a unit dose of 250 μg to 4 g. According to a more particular embodiment, the invention relates to a pharmaceutical composition as defined above, wherein said peptide construction is in a unit dose of 250 μg to 7 mg, 7 mg to 35 mg, 35 to 70 mg, 70 mg to 1.05 g, 1.05 g to 2.1 g, 2.1 g to 4 g.

The pharmaceutical composition of the invention may be administered by any suitable route of administration, for example parenterally, orally, sublingually, vaginally, rectally, transdermally, preferably by intravenous, subcutaneous or intradermal injection.

Intramuscular, intraperitoneal, intrasynovial, intrathecal or intratumoral injection is also possible. Injections can be performed as a bolus, or by continuous infusion.

Preparations for parenteral administration may include sterile aqueous or non-aqueous solutions, suspensions or emulsions. Examples of non-aqueous solvents are propylene glycol, polyethylene glycol, vegetable oils, such as olive oil, or injectable organic esters such as ethyl oleate. Aqueous vehicles include water, alcohol/water solutions, emulsions or suspensions.

According to a particular embodiment, the invention relates to a pharmaceutical composition as defined above, for an intravenous administration.

According to another particular embodiment, the invention relates to a pharmaceutical composition as defined above, for subcutaneous administration.

According to another particular embodiment, the invention relates to a pharmaceutical composition as defined above, for oral administration.

According to another particular embodiment, the invention relates to a pharmaceutical composition as defined above, for intravitreal administration. According to another particular embodiment, the invention relates to a pharmaceutical composition as defined above, for an intravenous, subcutaneous or oral administration, for its use in the treatment of congenital toxoplasmosis.

According to another particular embodiment, the invention relates to a pharmaceutical composition as defined above, for an intravitreal administration, for its use in the treatment of ocular toxoplasmosis.

The invention also relates to a composition comprising as active substance a peptide construct comprising the variable region of the heavy chain (VHH) of a first antibody recognizing the SAG1 antigen of Toxoplasma gondii and which may contain all or part of the constant region lacking a CH1 region, the heavy chain of a second antibody, said peptide construct recognizing the SAG1 antigen of Toxoplasma gondii and being capable of neutralizing the invasion of the cells by Toxoplasma gondii, optionally in association with a pharmaceutically acceptable vehicle. Said peptide construction is then devoid of variable light chain region. In this embodiment, said first antibody recognizing the SAG1 antigen of Toxoplasma gondii is a camelid antibody consisting of two heavy chains and lacking a light chain. The heavy chains of these antibodies comprise a variable domain (VHH) and a constant domain.

In another aspect, the invention relates to a peptide construct not containing a CH1 region, recognizing the SAG1 antigen of Toxoplasma gondii and capable of neutralizing the invasion of cells by Toxoplasma gondii, provided that said peptide construct is different from the sequence SEQ ID NO: 10.

The subject of the invention is a peptide construct comprising the variable region of the heavy chain of a first antibody recognizing the SAG1 antigen of Toxoplasma gondii and which may contain all or part of the constant region devoid of CH1 region, of the heavy chain of a second antibody, said peptide construct recognizing the SAG1 antigen of Toxoplasma gondii and being able to neutralize the invasion of the cells by Toxoplasma gondii, provided that said peptide construct is different from the sequence SEQ ID NO: 10.

According to a particular embodiment, the invention relates to a peptide construct comprising the variable region of the heavy chain of a first antibody recognizing the SAG1 antigen of Toxoplasma gondii and which may contain all or part of the constant region devoid of CH1 region of the heavy chain of a second antibody, all or part of the constant region devoid of CH1 region of the heavy chain of said second antibody making it possible to increase the half-life of said peptide construction under in vivo conditions, said peptide construct recognizing the SAG antigen of Toxoplasma gondii and being able to neutralize the invasion of the cells by Toxoplasma gondii, provided that said peptide construct is different from the sequence SEQ ID NO: 10.

The subject of the invention is a peptide construct comprising the variable regions of the heavy chain and of the light chain of a first antibody recognizing the SAG1 antigen of Toxoplasma gondii and capable of containing all or part of the constant region devoid of CH1 region of the heavy chain of a second antibody, said peptide construct recognizing the SAG1 antigen of Toxoplasma gondii and being capable of neutralizing the invasion of the cells by Toxoplasma gondii, provided that said peptide construct is different from the sequence SEQ ID NO: 10.

According to a particular embodiment, the invention relates to a peptide construct comprising the variable regions of the heavy chain and of the light chain of a first antibody recognizing the SAG1 antigen of Toxoplasma gondii and which may contain all or part of the constant region devoid of CH1 region, of the heavy chain of a second antibody, said all or part of the constant region devoid of a CH1 region of the heavy chain of said second antibody making it possible to increase the half-life of said peptide construct under in vivo conditions, said peptide construct recognizing the SAG1 antigen of Toxoplasma gondii and being able to neutralize the invasion of the cells by Toxoplasma gondii, provided that said peptide construct is different from the sequence SEQ ID NO: 10.

According to a particular embodiment, the invention relates to a peptide construct as defined above, wherein said second antibody is a murine IgG2a immunoglobulin, provided that said peptide construct is different from the sequence SEQ ID NO: 10.

According to a particular embodiment, the invention relates to a peptide construct as defined above, wherein said first antibody recognizing the SAG1 antigen of Toxoplasma gondii is the monoclonal antibody 4F11E12, provided that said peptide construct is different from the sequence SEQ ID NO: 10.

According to a particular embodiment, the invention relates to a peptide construct as defined above, recognizing the conformational epitope formed by the amino acids at positions 35 to 37, 39, 41, 42, 45, 48, 50, 59 at 65 and 112 to 114 of the amino acid sequence SEQ ID NO: 3, provided that said peptide construct is different from the sequence SEQ ID NO: 10.

According to a particular embodiment, the invention relates to a peptide construct as defined above, comprising a CDR having at least 95%, at least 96%, at least 97%, at least 98% or at least 99% of identity with the sequence SEQ ID NO: 4, provided that said peptide construct retains its ability to neutralize the invasion of the cells by Toxoplasma gondii and that said peptide construct is different from the sequence SEQ ID NO: 10.

According to a particular embodiment, the invention relates to a peptide construct as defined above, comprising a CDR having at least 95%, at least 96%, at least 97%, at least 98% or at least 99% of identity with the sequence SEQ ID NO: 5, provided that said peptide construct retains its ability to neutralize the invasion of the cells by Toxoplasma gondii and that said peptide construct is different from the sequence SEQ ID NO: 10.

According to a particular embodiment, the invention relates to a peptide construct as defined above, comprising a CDR having at least 95%, at least 96%, at least 97%, at least 98% or at least 99% of identity with the sequence SEQ ID NO: 6, provided that said peptide construct retains its ability to neutralize invasion of the cells by Toxoplasma gondii and that said peptide construct is different from the sequence SEQ ID NO: 10.

According to a particular embodiment, the invention relates to a peptide construct as defined above, comprising a CDR having at least 95%, at least 96%, at least 97%, at least 98% or at least 99% of identity with the sequence SEQ ID NO: 7, provided that said peptide construct retains its ability to neutralize the invasion of the cells by Toxoplasma gondii and that said peptide construct is different from the sequence SEQ ID NO: 10.

According to a particular embodiment, the invention relates to a peptide construct as defined above, comprising a CDR having at least 95%, at least 96%, at least 97%, at least 98% or at least 99% of identity with the sequence SEQ ID NO: 8, provided that said peptide construct retains its ability to neutralize the invasion of the cells by Toxoplasma gondii and that said peptide construct is different from the sequence SEQ ID NO: 10.

According to a particular embodiment, the invention relates to a peptide construct as defined above, comprising a CDR having at least 95%, at least 96%, at least 97%, at least 98% or at least 99% of identity with the sequence SEQ ID NO: 9, provided that said peptide construct retains its ability to neutralize the invasion of cells by Toxoplasma gondii and that said peptide construct is different from the sequence SEQ ID NO: 10.

According to a more particular embodiment, the invention relates to a peptide construct as defined above, comprising the following six CDRs:

a CDR1 having at least 95%, at least 96%, at least 97%, at least 98% or at least 99% identity with the sequence SEQ ID NO: 4,
a CDR2 having at least 95%, at least 96%, at least 97%, at least 98% or at least 99% identity with the sequence SEQ ID NO: 5,
a CDR3 having at least 95%, at least 96%, at least 97%, at least 98% or at least 99% identity with the sequence SEQ ID NO: 6,
a CDR4 having at least 95%, at least 96%, at least 97%, at least 98% or at least 99% identity with the sequence SEQ ID NO: 7,
a CDR5 having at least 95%, at least 96%, at least 97%, at least 98% or at least 99% identity with the sequence SEQ ID NO: 8, and a CDR6 having at least 95%, at least 96%, at least 97%, at least 98% or at least 99% identity with the sequence SEQ ID NO: 9, provided that said peptide construct retains its ability to neutralize the invasion of the cells by Toxoplasma gondii and that said peptide construct is different from the sequence SEQ ID NO: 10.

According to a more particular embodiment, the invention relates to a peptide construct as defined above, comprising a CDR consisting of the amino acid sequence SEQ ID NO: 4, provided that said peptide construct is different from the sequence SEQ ID NO: 10.

According to a more particular embodiment, the invention relates to a peptide construct as defined above, comprising a CDR consisting of the amino acid sequence SEQ ID NO: 5, provided that said peptide construct is different from the sequence SEQ ID NO: 10.

According to a more particular embodiment, the invention relates to a peptide construct as defined above, comprising a CDR consisting of the amino acid sequence SEQ ID NO: 6, provided that said peptide construct is different from the sequence SEQ ID NO: 10.

According to a more particular embodiment, the invention relates to a peptide construct as defined above, comprising a CDR consisting of the amino acid sequence SEQ ID NO: 7, provided that said peptide construct is different from the sequence SEQ ID NO: 10.

According to a more particular embodiment, the invention relates to a peptide construct as defined above, comprising a CDR consisting of the amino acid sequence SEQ ID NO: 8, provided that said peptide construct is different from the sequence SEQ ID NO: 10.

According to a more particular embodiment, the invention relates to a peptide construct as defined above, comprising a CDR consisting of the amino acid sequence SEQ ID NO: 9, provided that said peptide construct is different from the sequence SEQ ID NO: 10.

According to a still more particular embodiment, the invention relates to a peptide construct as defined above, comprising the following six CDRs:

a CDR1 consisting of the amino acid sequence SEQ ID NO: 4, a CDR2 consisting of the amino acid sequence SEQ ID NO: 5,
a CDR3 consisting of the amino acid sequence SEQ ID NO: 6,
a CDR4 consisting of the amino acid sequence SEQ ID NO: 7,
a CDR5 consisting of the amino acid sequence SEQ ID NO: 8 and
a CDR6 consisting of the amino acid sequence SEQ ID NO: 9,
provided that said peptide construct is different from the sequence SEQ ID NO: 10.

According to a particular embodiment, the invention relates to a peptide construct as defined above, devoid of the CH2 and CH3 regions of the aforementioned second antibody, provided that said peptide construct is different from the sequence SEQ ID NO: 10.

According to another particular embodiment, the invention relates to a peptide construct as defined above, comprising a CH3 region and devoid of CH2 region of the aforementioned second antibody, provided that said peptide construction is different from the sequence SEQ ID NO: 10.

According to another particular embodiment, the invention relates to a peptide construct as defined above, comprising a CH2 region and a CH3 region of the aforementioned second antibody, provided that said peptide construction is different from the sequence SEQ ID NO: 10.

According to another particular embodiment, the invention relates to a peptide construct as defined above, comprising a CH2 region and devoid of CH3 region of the aforementioned second antibody, provided that said peptide construction is different from the sequence SEQ ID NO: 10.

According to an advantageous embodiment, the invention relates to a peptide construct as defined above, chosen from: scFv, diabodies, single-chain diabodies, minibodies, such as scFv-CH3 and CH3-diabodies, scFv-Fc, diabody-Fc, provided that said peptide construct is different from the sequence SEQ ID NO: 10.

According to a particular embodiment, the invention relates to a peptide construct as defined above, comprising or consisting of a diabody consisting of two identical amino acid sequences having at least 90% identity, at least 91% d, identity, at least 92% identity, at least 93% identity, at least 94% identity, at least 95% identity, at least 96% identity, at least 97% identity at least 98% identity or at least 99% identity with the amino acid sequence SEQ ID NO: 11, provided that said peptide construct is different from the sequence SEQ ID NO: 10.

The two subunits of diabody are bound together by weak bonds, such as hydrophobic bonds, hydrogen bonds, ionic bonds or van der Waals bonds.

According to a more particular embodiment, the invention relates to a peptide construct as defined above, comprising or consisting of a diabody consisting of two amino acid sequences of sequence SEQ ID NO: 11.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 12, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 12.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 13, provided that said peptide construct is different from the sequence SEQ ID NO: 10.

According to a more particular embodiment, the invention relates to a peptide construct as defined above, comprising or consisting of a diabody consisting of two amino acid sequences of sequence SEQ ID NO: 13.

According to another particular embodiment, the invention relates to a peptide construct as defined above, comprising or consisting of a scFv-CH3 consisting of an amino acid sequence having at least 90% identity, at least 91% identity, at least 92% identity, at least 93% identity, at least 94% identity, at least 95% identity, at least 96% identity, at least 97% at least 98% identity or at least 99% identity with the amino acid sequence SEQ ID NO: 14, provided that said peptide construct is different from the sequence SEQ ID NO: 10.

According to a more particular embodiment, the invention relates to a peptide construct as defined above, comprising or consisting of a scFv-CH3 consisting of the amino acid sequence SEQ ID NO: 14.

According to another particular embodiment, the invention relates to a peptide construct as defined above, comprising or consisting of a diabody-CH3 consisting of two identical amino acid sequences having at least 90% identity, at least 91% identity, at least 92% identity, at least 93% identity, at least 94% identity, at least 95% identity, at least 96% identity, at least 97% at least 98% identity or at least 99% identity with the amino acid sequence SEQ ID NO: 15, provided that said peptide construct is different from the sequence SEQ ID NO: 10.

The two subunits of diabody-CH3 are bound together by weak bonds, such as hydrophobic bonds, hydrogen bonds, ionic bonds or van der Waals bonds.

According to a more particular embodiment, the invention relates to a peptide construct as defined above, comprising or consisting of a diabody-CH3 consisting of two amino acid sequences of sequence SEQ ID NO: 15.

According to another particular embodiment, the invention relates to a peptide construct as defined above, comprising or consisting of a scFv-Fc consisting of an amino acid sequence having at least 90% identity, at least 91% identity, at least 92% identity, at least 93% identity, at least 94% identity, at least 95% identity, at least 96% identity, at least 91% at least 98% identity) or at least 99% identity with the amino acid sequence SEQ ID NO: 16, provided that said peptide construct is different from the sequence SEQ ID NO: 10.

According to a more particular embodiment, the invention relates to a peptide construct as defined above, comprising or consisting of a scFv-Fc consisting of the amino acid sequence SEQ ID NO: 16.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 17, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 17.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 18, provided that said peptide construct is different from the sequence SEQ ID NO: 10.

According to a more particular embodiment, the invention relates to a peptide construct as defined above, comprising or consisting of a diabody consisting of two amino acid sequences of sequence SEQ ID NO: 18.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 19, provided that said peptide construct is different from the sequence SEQ ID NO: 10.

According to a more particular embodiment, the invention relates to a peptide construct as defined above, comprising or consisting of a diabody consisting of two amino acid sequences of sequence SEQ ID NO: 19.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 20, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 20.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 21, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 21.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 22, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 22.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 23, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 23.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 24, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 24.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 25, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 25.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 26, provided that said peptide construct is different from the sequence SEQ ID NO: 10.

According to a more particular embodiment, the invention relates to a peptide construct as defined above, comprising or consisting of a diabody consisting of two amino acid sequences of sequence SEQ ID NO: 26.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 27, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 27.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 28, provided that said peptide construct is different from the sequence SEQ ID NO: 10.

According to a more particular embodiment, the invention relates to a peptide construct as defined above, comprising or consisting of a diabody consisting of two amino acid sequences of sequence SEQ ID NO: 28.

According to another particular embodiment, the invention relates to a peptide construct as defined above, comprising or consisting of a scFv-CH3 consisting of an amino acid sequence having at least 90% identity, at least 91% identity, at least 92% identity, at least 93% identity, at least 94% identity, at least 95% identity, at least 96% identity, at least 97% at least 98% identity or at least 99% identity with the amino acid sequence SEQ ID NO: 29, provided that said peptide construct is different from the sequence SEQ ID NO: 10.

According to a more particular embodiment, the invention relates to a peptide construct as defined above, comprising or consisting of a scFv-CH3 consisting of the amino acid sequence SEQ ID NO: 29.

According to another particular embodiment, the invention relates to a peptide construct as defined above, comprising or consisting of a diabody-CH3 consisting of two identical amino acid sequences having at least 90% identity, at least 91% identity, at least 92% identity, at least 93% identity, at least 94% identity, at least 95% identity, at least 96% identity, at least 97% at least 98% identity or at least 99% identity with the amino acid sequence SEQ ID NO: 30, provided that said peptide construct is different from the sequence SEQ ID NO: 10.

According to a more particular embodiment, the invention relates to a peptide construct as defined above, comprising or consisting of a diabody-CH3 consisting of two amino acid sequences of sequence SEQ ID NO: 30.

According to another particular embodiment, the invention relates to a peptide construct as defined above, comprising or consisting of a scFv-Fc consisting of an amino acid sequence having at least 90% identity, at least 91% identity, at least 92% identity, at least 93% identity, at least 94% identity, at least 95% identity, at least 96% identity, at least 97% at least 98% identity or at least 99% identity with the amino acid sequence SEQ ID NO: 31, provided that said peptide construct is different from the sequence SEQ ID NO: 10.

According to a more particular embodiment, the invention relates to a peptide construct as defined above, comprising or consisting of a scFv-Fc consisting of the amino acid sequence SEQ ID NO: 31.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 32, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 32.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 33, provided that said peptide construct is different from the sequence SEQ ID NO: 10.

According to a more particular embodiment, the invention relates to a peptide construct as defined above, comprising or consisting of a diabody consisting of two amino acid sequences of sequence SEQ ID NO: 33.

According to another particular embodiment, the invention relates to a peptide construct as defined above, comprising or consisting of a diabody consisting of two identical amino acid sequences having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 34, provided that said peptide construct is different from the sequence SEQ ID NO: 10.

According to a more particular embodiment, the invention relates to a peptide construct as defined above, comprising or consisting of a diabody consisting of two amino acid sequences of sequence SEQ ID NO: 34.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 35, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 35.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 36, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 36.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 37, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 37.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 38, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 38.

According to another particular embodiment, the invention relates to a peptide construct as defined above, comprising or consisting of a scFv consisting of an amino acid sequence having at least 90% identity, at least 91% identity, at least 92% identity, at least 93% identity, at least 94% identity, at least 95% identity, at least 96% identity, at least 97% identity, at least 98% identity or at least 99% identity with the amino acid sequence SEQ ID NO: 39, provided that said peptide construct is different from the sequence SEQ ID NO: 10.

According to a more particular embodiment, the invention relates to a peptide construct as defined above, comprising or consisting of a scFv consisting of the amino acid sequence SEQ ID NO: 39.

The invention also relates to a peptide construct comprising the heavy chain variable region (VHH) of a first antibody recognizing the SAG1 antigen of Toxoplasma gondii and capable of containing all or part of the constant region devoid of CH1 region, of the chain, heavy second antibody, said peptide construct recognizing the SAG1 antigen of Toxoplasma gondii and being able to neutralize the invasion of cells by Toxoplasma gondii, provided that said peptide construct is different from the sequence SEQ ID NO: 10. Said peptide construction is then devoid of variable light chain region. In this embodiment, said first antibody recognizing the SAG1 antigen of Toxoplasma gondii is a camelid antibody consisting of two heavy chains and lacking a light chain. The heavy chains of these antibodies comprise a variable domain (VHH) and a constant domain.

The constructs of the present invention neutralize the invasion of cells by Toxoplasma gondii.

The in vitro neutralization of Toxoplasma gondii invasion of cells can be demonstrated by the following test: tachyzoïtes, of a RH strain of Toxoplasma gondii, are transfected with a plasmid encoding a marker of gene expression such as β-galactosidase. The β-galactosidase gene is under the control of the promoter of the gene encoding the SAG1 protein. A more detailed text is in point 5 of example 1.

The in vivo neutralization of Toxoplasma gondii invasion of cells can be demonstrated in a congenital toxoplasmosis model by the following test: mice are infected by gavage with cysts of Toxoplasma gondii strain 76k.

After the birth of the young mice, a follow-up of the protection as well as the induced immune response is carried out by measuring the parasite load in the brain and a cytokine assay (in particular interleukin 10 (IL-10) and interferon-gamma (IFNy)) on splenocytes. A more detailed text is in point 6.1 of example 1.

The in vivo neutralization of Toxoplasma gondii cell invasion can be demonstrated in a model of ocular toxoplasmosis by the following test of female mice are divided into different batches treated by intravitreal injection of tachyzoïtes of the strain of Toxoplasma gondii ME49 and/or peptide constructs. The eyes of the mice are then examined with a binocular magnifying glass in order to perform a clinical examination. A more detailed text can be found in point 6.2 of example 1.

The peptide constructs of the invention are capable of recognizing the SAG1 antigen of Toxoplama gondii because they possess at least one binding domain to the SAG1 antigen of Toxoplasma gondii. By “binding domain” is meant any site defined by the CDRs (or paratope) capable of binding to the epitope of a molecule with an affinity of less than 10⁻⁶ molar.

The definition of binding is by the indirect immunofluorescence assay as described in Section 4.3.4, Example 1. The binding of the peptide construct to the parasite is revealed by an antibody coupled to a fluorochrome.

### DESCRIPTION OF THE FIGURES

FIG. 1: FIG. 1 shows the analysis of the binding of the peptide construct to the parasite Toxoplasma gondii by immunofluorescence using a mouse anti-histidine primary antibody (diluted to 1/50) according to the supplier's recommendations) and a mouse anti-mouse IgG secondary antibody coupled to Alexa488 fluorochrome (diluted to 1/500 as recommended by the supplier). The SG0 peptide construct was added to cup-attached tachyzoïtes from a 160 μg/mL SG0 solution, the SG5 peptide construct from a 147 μg/mL SG5 solution, and the DbF3S2 peptide construct from a solution of DbF3S2 at 155 μg/mL. The observation was carried out under a fluorescence microscope (objective ×40). Parts A, C, E, G, and I correspond to phase-contrast tachyzoïte observations (white light) and parts B, D, F, H, and J correspond to immunofluorescence observations via antibody coupled to a fluorochrome.

A and B: tachyzoïtes alone.
C and D: tachyzoïtes with primary anti-histidine antibody and mouse anti-mouse IgG secondary antibody coupled to Alexa488 fluorochrome.
E and F: tachyzoïtes with the SG0 peptide construct.
G and H: tachyzoïtes with the SG5 peptide construct.
I and J: tachyzoïtes with the peptide construct DbF3S2.

FIG. 2A: FIG. 2A shows the neutralizing activity of the SG0 peptide construct on the cellular invasion of Toxoplasma gondii tachyzoïtes.

HFF cells were infected with tachyzoïtes transfected with a plasmid encoding β-galatosidase. The peptide constructs (1.6 μg of SG0, in a volume of 50 μL prepared from a solution of 10⁹ μg/mL according to paragraph 5. In vitro neutralization test of Example 1 (Materials and Methods), or 1.6 μg of B6P (irrelevant antibody), in a volume of 50 μL prepared from a solution of 113 μg/mL, according to paragraph 5. In vitro neutralization test of Example 1 (Materials and Methods)) were added simultaneously to the parasites (100 tachyzoïtes). The graph represents the β-galactosidase activity measured spectrophotometrically at 565 nm for each condition.

HFF cells (HFF alone) constitute the negative control of cellular invasion of the parasite and HFF cells in the presence of 100 tachyzoïtes of Toxoplasma gondii (100 Tachys) constitute the positive control.

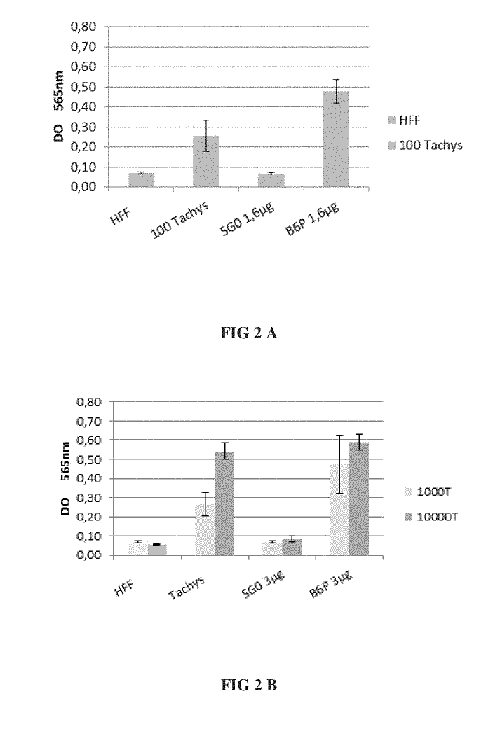

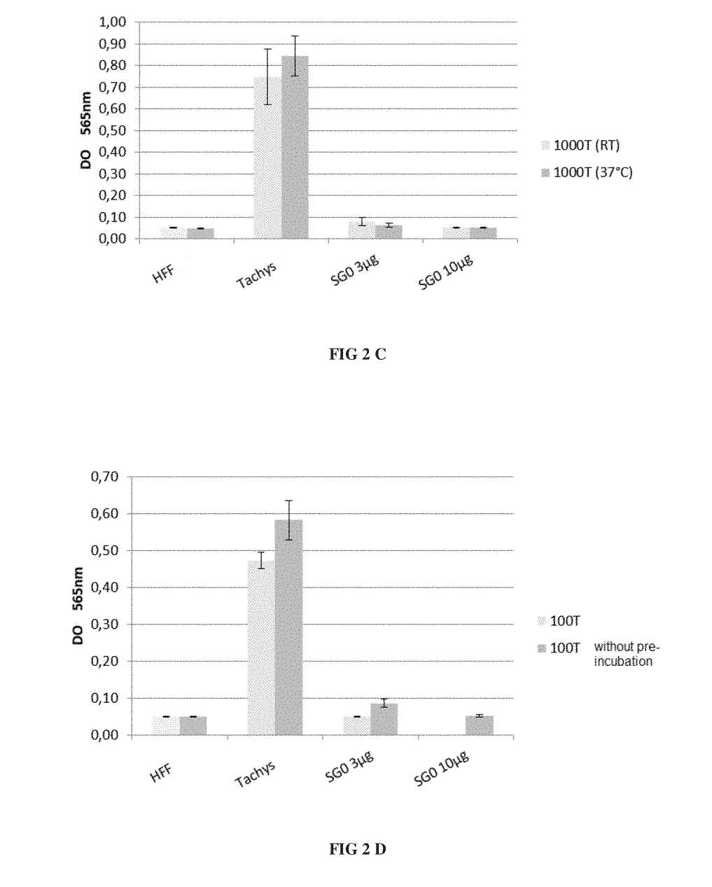

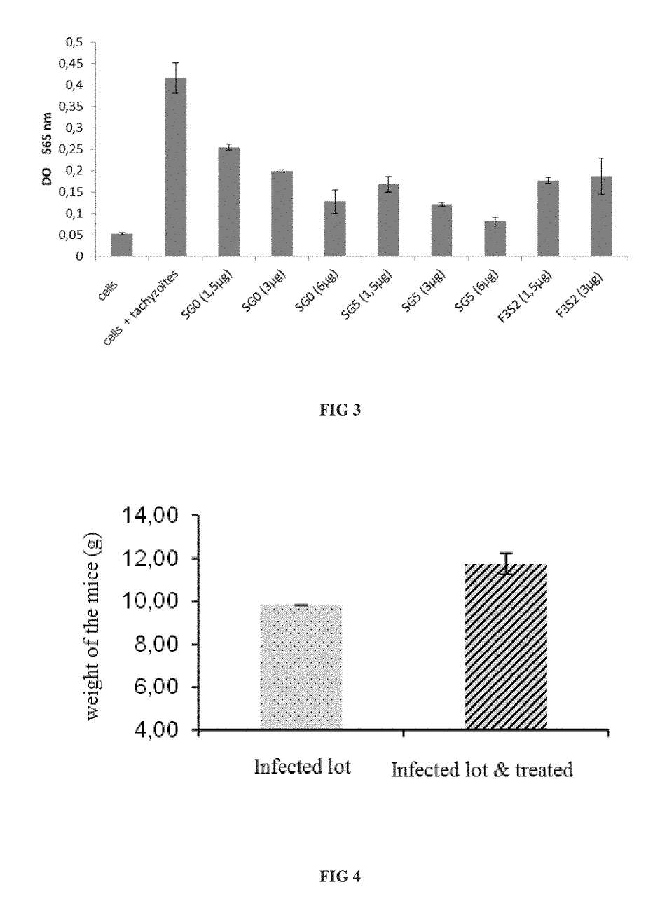

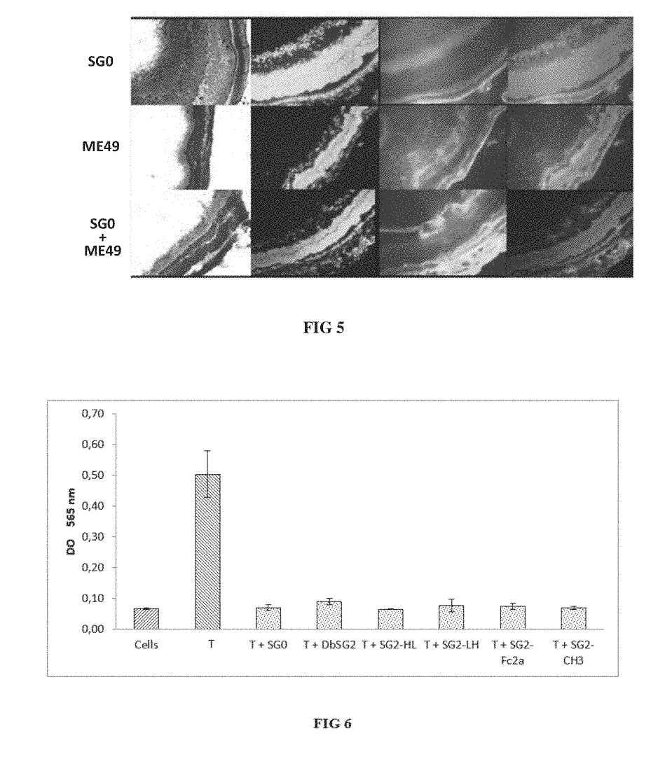

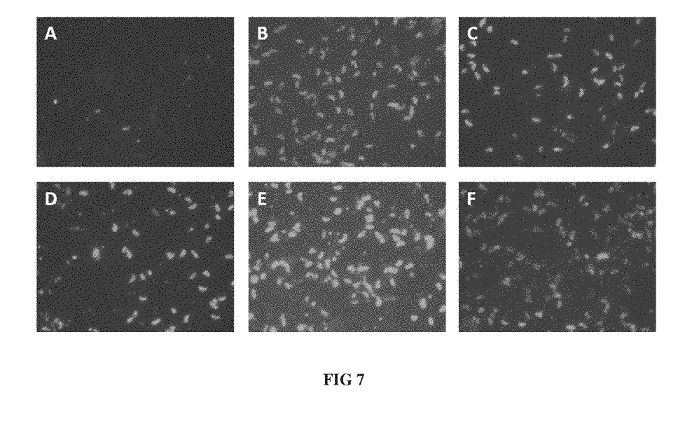

FIG. 2B: FIG. 2B shows the neutralizing activity of the SG0 peptide construct on the cellular invasion of Toxoplasma gondii tachyzoïtes.

HFF cells were infected with tachyzoïtes transfected with a plasmid encoding β-galatosidase. The peptide constructs (3 μg of SG0, in a volume of 50 μl prepared from a solution of 109 μg/mL according to paragraph 5. In vitro neutralization test of Example 1 (Materials and Methods), or 3 μg of B6P (irrelevant antibody), in a volume of 50 μL, prepared from a solution of 113 μg/mL according to paragraph 5. In vitro Neutralization Test of Example 1 (Materials and Methods)) were added simultaneously to the parasites. Two doses of parasites were used: 1000 tachyzoïte (1000T) and 10,000 tachyzoïtes (10000T). The graph represents the β-galactosidase activity measured spectrophotometrically at 565 nm for each condition. HFF cells (HFF alone) constitute the negative control of cell invasion of the parasite and HFF cells in the presence of 1000 or 10,000 Toxoplasma gondii tachyzoïtes (Tachys alone) constitute the positive control.

FIG. 2C: FIG. 2C represents the neutralizing activity of the SG0 peptide construction on the cellular invasion of the tachyzoïtes of Toxoplasma gondii, as a function of the incubation temperature of the HFF cells with the tachyzoïtes and the peptide construction (incubation of 4 days): room temperature (1000T (RT)) or 37° C., physiological temperature (1000T (37° C.)). HFF cells were infected with tachyzoïtes transfected with a plasmid encoding β-galatosidase. 3 μg or 10 μg of SG0, in a volume of 50 μl, prepared from a solution of 109 μg/mL according to paragraph 5. In vitro neutralization test of Example 1 (Materials and Methods), were added simultaneously to the parasites (1000 tachyzoïtes). The graph represents the β-galactosidase activity measured spectrophotometrically at 565 nm for each condition.

HFF cells (HFF alone) are the negative control of cellular invasion of the parasite and HFF cells in the presence of 1000 tachyzoïtes Toxoplasma gondii (Tachys alone) constitute the positive control.

FIG. 2 D: FIG. 2 D shows the neutralizing activity of the peptidic SG0 construct, in increasing doses, on the cellular invasion of Toxoplasma gondii tachyzoïtes. HFF cells were infected with tachyzoïtes transfected with a plasmid encoding β-galatosidase. SG0 (3 μg or 10 μg in a volume of 50 μl prepared from a solution of 109 μg/mL according to paragraph 5. In vitro neutralization test of Example 1 (Materials and Methods)) was added to the HFF cells either simultaneously with the parasites (100 tachyzoïtes), after a pre-incubation of 1 h with the tachyzoïtes (100T), or without pre-incubation, with the tachyzoïtes, with a delay of 5 min after the addition of tachyzoïtes (100T no pre-incubation). The graph represents the β-galactosidase activity measured spectrophotometrically at 565 nm for each condition.

HFF cells (HFF alone) constitute the negative control of cellular invasion of the parasite and HFF cells in the presence of 100 Toxoplasma gondii tachyzoïtes (Tachys alone) constitute the positive control.

FIG. 3: FIG. 3 shows the dose-dependent effect of peptide constructs on their neutralizing activity on the cellular invasion of Toxoplasma gondii tachyzoïtes.

HFF cells were infected with tachyzoïtes transfected with a plasmid encoding β-galatosidase. The peptide constructs (1.5 μg, 3 μg and 6 μg of SG0 (in a volume of 50 μL, prepared from a solution of 160 μg/mL according to paragraph 5. In vitro Neutralization Test of Example 1 (Materials and Methods); 1.5 μg, 3 μg and 6 μg of SG5 (in a volume of 50 μL, prepared from a solution of 147 μg/mL according to paragraph 5. In vitro Neutralization Test of Example 1 (Materials and Methods); 1.5 μg and 3 μg of DbF3S2 (in a volume of 50 μL, prepared from a solution of 155 μg/mL according to paragraph 5. In vitro Neutralization Test of Example 1 (Materials and Methods)) were added simultaneously to the parasites. The graph represents the 1-galactosidase activity measured spectrophotometrically at 565 nm for each condition.

HFF cells (cells) constitute the negative control of the cellular invasion of the parasite and HFF cells in the presence of 100 tachyzoïtes of Toxoplasma gondii (cells+tachyzoïtes) constitute the positive control.

FIG. 4: FIG. 4 represents the average weight of the mice in gram 3 weeks after birth according to the treatment received by the mother.

Infected lot: the pregnant mice were infected by gavage with 10 cysts of the strain 76K of Toxoplasma gondii at day 11.

Batch infected and treated: the pregnant mice were infected by gavage with 10 cysts of the strain 76K of Toxoplasma gondii at day 11 and then treated with a daily intraperitoneal injection of 15 μg of SiO (from a solution of SG0 at 120 μg/mL) until farrowing.

FIG. 5: FIG. 5 shows the immunolabeling of tackyzoites carried out on retinal sections of mouse eyes in which SG0 alone (at a concentration of 120 μg/mL) (SG0 line) or tachyzoïtes of the strain ME49 alone (line ME49) or tachyzoïtes+SG0 (line SG0+ME49). The observation was performed under a fluorescence microscope (×250 magnification). In line 2 (ME49), the tachyzoïtes are visible by immuno-labeling in column 3. In the presence of SG0 (line SG0+ME49) the tachyzoïtes are no longer detectable in immuno-labeling, which underlines the neutralizing effect of the antibody on the cellular invasion of the parasite.

FIG. 6: FIG. 6 shows the neutralizing activity of peptide constructs SG0. SG2-HL, SG2-LH, DbSG2, SG2-Fc2a and SG2-CH3 on the cellular invasion of Toxoplasma gondii tachyzoïtes.

HFF cells were infected with tachyzoïtes transfected with a plasmid encoding β-galatosidase. Peptide constructs (5 μg of SG0, in a volume of 50 μL, prepared from a solution of 125 μg/mL, or 5 μg of SG2-HL, in a volume of 50 μL, prepared from a solution at 75 μg mL, or 5 μg of SG2-LH, in a volume of 50 μl, prepared from a solution of 90 μg/mL, or 5 μg of DbSG2, in a volume of 50 μM, prepared from of a solution at 80 jμg/mL, or 5 μg of SG2-Fc2a, in a volume of 50 μl, prepared from a solution of 85 μg/mL, or 5 μg of SG2-CH3, in a volume of 50 μl, prepared from a 75 μg/mL solution, according to paragraph 5. In vitro neutralization test of Example 1 (Materials and Methods), were added simultaneously to the parasites. A dose of 1000 tachyzoïte parasites was used. The graph represents the β-galactosidase activity measured spectrophotometrically at 565 nm for each condition.

HFF cells (HFF alone) constitute the negative control of the cellular invasion of the parasite and HFF cells in the presence of 1000 tachyzoïtes of Toxoplasma gondii (T alone) constitute the positive control.

FIG. 7: FIG. 7 shows the analysis of the binding of different peptide constructs to the parasite Toxoplasma gondii by immunofluorescence using a mouse anti-histidine primary antibody (diluted to 1/50) according to the supplier's recommendations) and a mouse anti-mouse IgG secondary antibody coupled to Alexa488 fluorochrome (diluted to 1/500 as recommended by the supplier). The peptide construct DbSG2 was added to the tachyzoïtes fixed on the wells with a solution of DbSG2 at 80 μg/mL, the peptide construct SG2-HL from a solution of SG2-HL at 75 μg/mL, the SG2-LH peptide construct from a solution of SG2-LH at 90 μg/mL, the SG2-Fc2a peptide construct from a solution of SG2-Fc2a at 85 μg mL, and the peptide construct SG2-CH3 at from a solution of SG2-CH3 at 75 μg/mL.

The observation was carried out under a fluorescence microscope (objective ×40). The observations are made by immunofluorescence using an antibody coupled to a fluorochrome.

A: negative control, tachyzoïtes in the presence of induced S2 cell supernatant.
B: tachyzoïtes with the peptide construct DbSG2 at 80 μg/mL.
C: tachyzoïtes with the peptide construct SG2-HL at 75 μg/mL.
D: tachyzoïtes with the peptide construct SG2-LH at 90 μg/mL.
E: tachyzoïtes with the SG2-Fc2a peptide construct at 85 μg/mL.
F: tachyzoïtes with the peptide construct SG2-CH3 at 75 μg/mL.

### EXAMPLES

### Example 1: Materials and Methods

1. Construction of the Expression Vectors

1.1. Bacteria and Plasmids Used

1.1.1. Bacteria and Plasmids Used for Expression of SG5 and SG0 Peptide Constructs

The bacteria Escherichia coli BL21 of genotype F-, ompT, hsdSB (r_{B-}, m_{B-}), dcm, gal, λ(DE3), pLysS, Cm^{r} were used for the expression of the peptide constructs from the plasmid vector pET22b (NOVAGEN) (prokaryotic system), under the control of the T7 promoter.

The expression vector pET22b contains a pelB sequence allowing the export of the peptide constructs in the periplasm of the bacteria, an oxidizing domain making it possible to obtain correctly conformed peptide constructs. This sequence was fused with the VH and VL domain coding sequences of the peptide constructs, as well as the sequence of a peptide link. The plasmids also have a coding sequence for a histidine tag, facilitating the purification of the protein by affinity chromatography as well as its detection.

For the SG5 peptide construct, the pGEMT cloning vector (PROMEGA) was first used. The latter allowed the direct cloning of PCR products. Indeed, the plasmid pGEMT is linearized with the restriction enzyme EcoRV and has a thymidine at each of its ends for binding the adenine added to the amplicons by the Taq polymerases without exonuclease activity. This 3015 bp plasmid encodes, in particular, β-lactamase (conferring on the host bacteria an ampicillin resistance) and comprises the β-galactosidase α-subunit gene (lacZ cassette). In the latter is located the multiple cloning site framed by the promoters T7 and SP6. The β-galactosidase gene thus allows a “blue-white” screening of colonies possessing the recombinant plasmid on Lysogeny Broth (LB) agar medium supplemented with IPTG, X-gal and ampicillin: the bacteria having integrated the plasmid recombinant remain white while bacteria lacking the recombinant plasmid are stained blue.

The bacteria Escherichia coli TG1 of genotype supE thi-1 Δ(lac-proAB) Δ(mcrB-hsdSM5 (rK-mK-) [F′ traD36 proAB lacI^{q} Z Δ M15] were used for plasmid cloning.

1.1.2. Bacteria and Plasmids Used for the Expression of scFvSG1 Peptide Constructs. SG2-HL. DbF3S2, DbF4S2, scFvF5S2. scFvF5S4. scFvF5S5. scFvF5S6 and Hum-scFvH2S

The bacteria Escherichia coli HB2151 of genotype K12, ara, Δ(lac-pro), thi(F′proAB, lacI^{q} lacZ Δ M15) were used for the expression of the peptide constructs scFvSG1. SG2-HL, DbF3S2, DbF4S2, scFvF5S2, scFvF5S4, scFvF5S5, scFvF5S6 and Hum-scFvH2S from plasmid vector pSW1 (PMID: 23680984) (prokaryotic system). These plasmids also have: i) modifications at the level of the VL allowing purification by protein-L (PpL), and ii) a coding sequence for a histidine tag that can be used for the purification of the protein by affinity chromatography as well as for its detection.

1.1.3. Bacteria and Plasmids Used for the Expression of SG2-LH Peptide Constructs. DbSG2. SG2-CH3. SG2-Fc2a. SG2-HL and DbSG2-CH3

The bacteria Escherichia coli DH5a of genotype F′/endA1, hsdR17, (rk− mk+), supE44, thi-1, recA1, gyrA, (Nal1r), relA1, Δ(lacZYA-argF) U169, deoR, (Φ80dlacΔ (lacZ) M15) were used for the expression of peptide constructs from the plasmid vector pMT-Bip (ThermoFischer) (eukaryotic system) under the control of the metallothionein promoter. This step aims to produce a sufficient amount of plasmids (containing an insert) before carrying out the transfection of eukaryotic cells with said plasmids.

The pMT-Bip expression vector contains a BiP sequence allowing the secretion of peptide constructs in the cell culture supernatant. This sequence was fused with the VH and VL domain coding sequences of the peptide constructs, as well as the sequence of a peptide link. In addition, these plasmids possess:

i) modifications at the level of the VL domain allowing purification by PpL, and
ii) a coding sequence for a histidine tag that can be used for the purification of the protein by affinity chromatography as well as for its detection.

1.2. Purification of Bacterial Plasmids

1.2.1 Plasmids pET22b and pSW1 and pGEMT

The bacteria (BL21 and HB21) were cultured for 16 hours at 37° C. in 5 mL of LB medium containing ampicillin (50 μg/mL). The plasmids were then extracted and purified using the Plasmid Minikit I kit (Omega Biotek) according to the conditions mentioned by the supplier. The method used is that of alkaline lysis.

1.2.2. PMT-BIP Plasmids

The bacteria (DH5a) were cultured for 16 hours at 37° C. in 100 mL of LB (Luria Broth) medium containing ampicillin (50 μg/mL). The plasmids were then extracted and purified using the Plasmid Maxi Kit kit (QIAGEN) according to the conditions mentioned by the supplier. The method used is that of alkaline lysis.

1.3. Techniques Used for the Construction of Recombinant Plasmids

1.3.1. Gene Amplification by Polymerase Chain Reaction (PCR) for the SG5

The amplification of the nucleotide sequence coding for the variable domain of the VL light chain of the SG0 peptide construct was carried out by means of a PCR. The reaction consisted of a succession of 27 cycles consisting of a denaturation phase of the DNA (95° C.), a hybridization phase with the two primers of sequence SEQ ID NO: 70 and sequence SEQ ID NO: 71 presented in Table 1 (50° C.) and an extension phase by DNA polymerase from the primers (72° C.). The amplification reactions were carried out in a final volume of 50 μL containing 0.25 units of GoTaq polymerase (Promega), 0.1 μL of each primer, 0.1 mM of each dNTP, 10 μL of 5× GoTaq buffer. Flexi Buffer (Promega) and 1 μg of template DNA. The “sense” primer (SG5For) of sequence SEQ ID NO: 70 made it possible to introduce a BamHI site at the N-terminus of the amplified VL domain, and the sequence “antisense” primer (SG5rev). SEQ ID NO: 71 introduced a XhoI site at the C-terminus.

1.3.2. Cloning of PCR Products

The PCR products were purified (using a Macherey Nagel Extraction Kit) and cloned using the pGEM-T vector (Promega). Their ligation was performed in a final volume of 10 μL containing 2 μg of PCR products, 100 ng of pGEM-T vector and 1 unit of T4 DNA ligase. The ligation was carried out at room temperature for 1 h, then the reaction mixture was used for the transformation of TG1 bacteria. The transformed bacteria were selected on LB medium (peptone 10 g/L, yeast extract 5 g/L and NaCl 10 g/L) supplemented with 50 μg/mL ampicillin, 10 μM X-gal and 100μ IPTG.

1.3.3. Enzymatic Digestion of DNA

The digests of the inserts (protein coding sequences of the SG0, SG2-LH, DbSG2, SG2-CH3 SG2-Fc2a, SG2-HL, DbSG2-CH3, scFvSG1, DbF3S2, DbF4S2, scFvF5S2, scFvF5S4 and Hum-scfvH2S peptide constructs and sequence coding the VL domain of the peptide construct SG0) as well as plasmids (pET22b, PMT-Bip, pSW1, pGEMT) were carried out using different pairs of endonucleases, using 10 units of enzymes per μg of DNA in the presence of the recommended buffer. The reactions were incubated for 15 min at 37° C. The pairs of endonucleases used for each peptide construct and the expression vectors in which the inserts are subsequently cloned are shown in Table I.

TABLE I

| Names | | | | |
|---|---|---|---|---|
| Peptidic | | | | |
| Construc- | | | Enzymatic | Expression |
| -tions | Format | PCR (5′→3') | Digestion | vector |
| SG5 | diabody | SG5 For SEQ ID NO: 70 | BamHI/ | pET22b |
| | | (GGATCCCGATATTCAGGTTACCAG) | XhoI | |
| | | SG5Rev, of SEQ ID NO: 71 | | |
| | | (TTTCTCGAGTTAGTGATGGTGATG) | | |
| SG0 | scFv | Synthesis Genes | PstI/XhoI | pET22b |
| SG2-LH | scFv | | EcoRV/XhoI | pMT-Bip |
| DbSG2 | Diabody | | PstI/XhoI | pMT-Bip |
| SG2-CH3 | scFv-CH3 | | PstI/XhoI | pMT-Bip |
| SG2-Fc2a | scFv-Fc | | KpnI/XhoI | pMT-Bip |
| SG2-HL | scFv | | PstI/XhoI | pMT-Bip |
| DbSG2- | Diabody- | | KpnI/XhoI | pMT-Bip |
| CH3 | CH3 | | | |
| scFvSG1 | scFv | | PstI/XhoI | pSW1 |
| SG2-HL | scFv | | PstI/XhoI | pSW1 |
| DbF3S2 | Diabody | | PstI/XhoI | pSW1 |
| DbF4S2 | Diabody | | PstI/XhoI | pSW1 |
| scFvF5S2 | scFv | | PstI/XhoI | pSW1 |
| scFvF5S4 | scFv | | PstI/XhoI | pSW1 |
| scFvF5S5 | scFv | | PstI/XhoI | pSW1 |
| scFvF5S6 | scFv | | PstI/XhoI | pSW1 |
| Hum- | scFv | | PstI/XhoI | pSW1 |
| scfvH2S | | | | |

1.3.4. DNA Electrophoresis

The DNA fragments were separated by agarose gel electrophoresis (1.5-2%) in TAE buffer (40 mM Tris-Acetate, 1 mM EDTA pH 8). The gels were supplemented with 0.1 μg/mL of ethidium bromide (BET), which made it possible, after migration under a current of 100 V, to visualize the DNA under ultraviolet radiation. BenchTop and lkD (Promega) size markers were used to determine the size of the fragments analyzed.

1.3.5. Purifications of DNA Fragments from Agarose

After electrophoretic migration, the agarose bands containing the DNA fragments resulting from the enzymatic digestions were excised and purified using the Nucleospin® Extract II kit (Macherey-Nagel®) according to the conditions mentioned by the supplier. The principle of purification is based on the DNA affinity for silica in the presence of high sodium concentration. A volume of DNA, to be purified, supplemented with two volumes of a “binding” solution (Binding Buffer NT) was deposited on a silica membrane. While the DNA fragments larger than 50 bp are membrane-bound, the dNTPs, oligonucleotides and proteins present in the PCR product were removed by centrifugation (11,000 g, 1 min). The membrane was then cleaned of remaining salts and proteins by washing with 700 of lower salinity NT3 buffer containing ethanol (centrifugation at 11,000 g, 1 min). It was then dried by centrifugation (2 min at 11,000 g).

The purified DNA retained by the column was finally eluted in milliQ water by centrifugation (11,000 g, 1 min).

1.3.6. Ligation Vector-Insert

The ligation was performed in a final volume of 10 μL of ligation medium (consisting of ligase buffer and water) containing 1 unit of T4 DNA ligase (Promega®) and 200 ng of pGEMT vector, pET22b, pSW1 or pMT.-Bip (previously digested). The amount of insert was adjusted so that the vecteuninsert molar ratio was 1:3. The reactions were then incubated overnight at 4° C.

1.4. Transformation of Competent Bacteria

1.4.1. Preparation of Competent Bacteria

The bacteria were made competent by CaCl₂) treatment. The bacteria were first cultured to the exponential growth phase (A_{600 nm}=0.4) in LB medium. Then, the culture was placed at 4° C. for 10 min and then centrifuged (20 min, 3000 g, 4° C.). The bacterial pellet was resuspended with 10 mL of a CaCl₂ solution. at 0.1M and put in the ice (4° C.) for about 1:30. Centrifugation (20 min, 3000 g, 4° C.) recovered the bacterial pellet which was resuspended in 2 mL of 0.1M CaCl₂) solution and then kept at 4° C. in ice until use.

1.4.2. Bacterial Transformation

5 μL of the ligation product was added to 200 μL of competent bacteria. The mixture was incubated for 30 min at 4° C. Thermal shock was achieved by placing the sample 90 seconds at 42° C. and then 2 min at 4° C. The bacteria were put back into culture by adding 800 μL of LB medium for 45 min at 37° C. with shaking (200 rpm).

Only the bacterial transformation with the plasmid pGEMT required the prior addition of X-Gal and IPTG (0.84 mM) on LB agar/ampicillin agar (1 μg/μL) thus allowing a “white” screening, blue”. Subsequently, the protocol was similar for all the transformations: 200 μL of the transformation was plated on LB agar/ampicillin agar. The dishes were then incubated overnight at 37° C.

2. Protein Production

2.1. Protein Expression in Bacterial System

2.1.1. Bacterial Strain: Escherichia coli

The expression system that was chosen to produce the recombinant proteins required Escherichia coli bacterial strains BL21pLysS and/or HB2151, depending on the vectors used.

2.1.2. Bacterial Production

The peptide constructs were produced in the periplasm of the transformed bacteria Escherichia coli BL21pLysS or HB2151 by the different plasmids.

Preculture of the bacteria was performed in 5 mL of LB medium and incubated overnight at 37° C. with shaking (200 rpm). A volume of 500 μL of this preculture was used to seed 500 mL of 2×YT medium containing 50 tμg/mL of ampicillin. Then the culture was incubated for 8 h at 37° C. with shaking (150 rpm). Induction of the production of the peptide constructs was achieved by the addition of 0.84 mM IPTG, and the bacterial culture was incubated overnight (16° C., shaking 75 rpm).

2.1.3. Periplasmic Extraction

The proteins produced were extracted from the periplasm by osmotic shock. The IPTG-induced culture was centrifuged (5000 g, 10 min, 4° C.). The pellet was taken up in 10 mL of TES buffer (0.2M Tris pH8, 0.5 mM EDTA, 0.5M sucrose) and incubated for 30 minutes in ice. Then 15 mL of one quarter diluted TES was added and the solution was incubated for 30 min in ice. Further centrifugation (10,000 g, 10 min, 4° C.) removed cell debris and recovered the periplasm containing the protein of interest. The supernatant was then dialyzed against PBS buffer (Phosphate Buffered Saline: 27 mM KCl, 1.4 mM NaCl, 15 mM KH₂PO₄ anhydrous, 80 mM Na₂HPO₄, pH 7.4).

2.2. Expression of Proteins in the Eukaryotic System

Proteins were produced in Schneider Drosophila S2 insect cells (SD-S2). After thawing, the cells were cultured in S2 medium (Schneider Drosophila 2 medium, 10% fetal calf serum, Penicillin-Streptomycin 1%). Transfection of the cells was performed with the Lipofectamine® 2000 kit (LifeTechnology®) according to the manufacturer's recommendations. Thus, these cells were first transfected transiently with the plasmid pMT-Bip containing the sequences of interest. The supernatants were analyzed 4 days later on 12% acrylamide gel by Western blot after transfer on nitrocellulose membrane to ensure that the proteins were well produced. In parallel, SD-S2 cells were stably transfected with the plasmid pMT-Bip containing the sequences of interest and the plasmid pMT-Bip containing a hygromycin resistance gene. The transfected cells were selected by resistance to hygromycin (300 μg/mL) added to the S2 culture medium. Induction of protein production was feasible as soon as the cells reached sufficient multiplication kinetics after about three weeks of selection.

A production cycle is organized as follows: the cells were cultured in S2-hygromycin medium for 5 days after which the cells were recovered by centrifugation (700 g, 10 min, 30° C.), repeated in 10 mL of Schneider Drosophila medium, then counted with Trypan Blue on Malassez cell. The cells were then reintroduced into 225 cm 3 culture flasks in induction medium (Schneider Drosophila 2 Medium, Penicillin-Streptomycin 1%, CuSO₄ 1 mM) so as to obtain 400 mL of cells at a density of 5·10⁶ cells/mL. Copper sulfate activates transgene expression through the metallothionein promoter. Culture supernatants were removed 96 h after induction and then centrifuged (700 g, 10 min, 30° C.) to remove the cells. Part of the transfected cells was not induced and was re-cultured to begin a new cycle of recombinant protein production.

3. Purification of Proteins

3.1. Purification of Recombinant Proteins by Nickel-Agarose Column Affinity Chromatography

The purification of the recombinant proteins is based on the affinity of the nickel atoms with the histidine residues of the C-terminal label of the protein. First, in order to remove as much debris as possible, the periplasm was centrifuged for 10 min at 5,000 g at room temperature. 300 mL of nickel-agarose gel (Miltenyi Biotec) per 50 mL of periplasm were added to the supernatant and then the solution was stirred slowly for 1 h 30 min.

Centrifugation for 3 min at 500 g allowed to harvest all the gel, which was loaded on a chromatography column. Then, washing was carried out with 25 mL of PBS and then 5 mL of a glycine solution (0.1M, pH 6). Elution of the protein was performed with 5 mL of a 0.1M glycine solution at pH 3. Fractions of 1 mL were recovered and immediately neutralized by addition of 1M Tris. They were then dialyzed against a PBS buffer at 4° C. overnight.

After dialysis, the absorbance at 280 nm was measured in order to estimate the protein concentration according to the Beer-Lambert law A=εlC (A: absorbance, ε: molar extinction coefficient, l: length of the tank; C: concentration).

3.2. Purification of Recombinant Proteins by Affinity Chromatography on PpL-Agarose Column

First, in order to remove as much debris as possible, the bacterial periplasm or cell culture supernatant was centrifuged for 10 min at 5,000 g at room temperature. 300 μL of agarose per 50 mL of periplasm or culture supernatant was added to the centrifugation supernatant, and then the solution was stirred slowly for 1 h 30 min.

A centrifugation of 3 min at 500 g allowed to harvest all the gel, which was loaded on a chromatography column. Then, washing was carried out with 25 mL of PBS and then 5 mL of a glycine solution (0.1M, pH 6). The elution of the protein was carried out with 5 mL of a 0.1M glycine solution at pH 3 and then with 5 mL of a 0.1M glycine solution at pH 2.

Fractions of 1 mL were recovered and immediately neutralized by addition of 1M Tris. They were then dialyzed against a PBS buffer at 4° C. overnight. After dialysis, the absorbance at 280 nm was measured in order to estimate the protein concentration according to the Beer-Lambert law A=εlC (A: absorbance, ε: molar extinction coefficient, l: length of the tank; C: concentration).

4. Protein Analysis

4.1. Structural Analysis: FPLC (Fast Protein Liquid Chromatography

The purifications of the peptide constructs were analyzed by exclusion-diffusion chromatography on a Superdex column (Biosciences). 200 μL of peptide constructs (fractions collected after purification by chromatography on a nickel-agarose column or on a PpL-agarose column) were loaded onto the column.

Protein elution was performed with PBS at a flow rate of 0.5 mL/min before performing an absorbance measurement at 280 nm.

4.2. Electrophoresis in Polyacrylamide Gel (SDS-PAGE) Under Denaturing Conditions

Protein samples (fractions collected after purification by chromatography on a nickel-agarose column or on a PpL-agarose column) were diluted in loading buffer (Tris-HCl pH 6.8 100 mM, SDS (Sodium Dodecyl Sulfate) 4%, β-1% mercapto-ethanol, 0.2% bromophenol blue, 20% glycerol) and then denatured by heating at 95° C. for 5 min. They were then deposited on a 12% polyacrylamide gel and were then migrated under a current of 60 mA. The proteins present in the gel were visualized after 30 min staining with Coomassie blue (0.25% Coomassie blue, 45% methanol and 10% acetic acid) and bleaching with the discoloration buffer.

4.3. Characterization of Proteins by Immuno-Detection

4.3.1. Antibodies Used for Protein Detection

An anti-histidine monoclonal antibody (Sigma) was used to detect the presence of proteins with the histidine tag. An alkaline phosphatase-coupled murine anti-IgG antibody (Sigma) was used as a secondary antibody to reveal the presence of the anti-histidine monoclonal antibody. HRP (Horse Radish Peroxidase) -induced anti-histidine antibody (Miltenyi Biotec) was used to directly reveal the presence of proteins with the histidine Lag. HRP (Horse Radish Peroxidase) coupled L (PpL) protein is used to directly reveal the presence of proteins recognized by protein L.

4.3.2. Western Blot

Passive protein transfer on nitrocellulose membrane was performed overnight. The next day, the membrane was first saturated with a TNT solution (15 mM Tris-HCl, 140 mM NaCl pH 8, 0.05% Tween 20) supplemented with 5% skim milk powder for 1 h.

Peptide Constructs with the Tag Histidine:

Anti-histidine monoclonal antibody was added to the membrane at 1/2000 dilution for 1 h while stirring. The membrane was washed three times with TNT and then incubated with the secondary murine anti-mouse IgG antibody coupled to alkaline phosphatase (diluted 1/5000 in TNT) for 1 hour with shaking. The washing of the membrane was carried out twice with TNT then with buffer R (100 mM Tris-HCl, 100 mM NaCl 5 mM MgCl₂ H₂O, pH 9.5). The presence of the antibodies was revealed by the reaction of the alkaline phosphatase with its substrate (BCIP: Bromo Chloro Indolyl Phosphate) in the presence of NBT (Nitro Blue Tetrazolium) and the color reaction was stopped by rinsing with distilled water.

4.3.3. ELISA

A 96-well plate (NUNC Maximax) was coated with 10 μg/mL of toxoplasma total extract (ET) diluted in carbonate buffer (0.1M Na₂CO₃, NaHCO₃, pH 9.6) at 100 μL/well. The plate was left overnight at 4° C.

The next day, the saturation of the non-specific sites was carried out by 200 μL/well of PBS-Tween supplemented with 3% of BSA (Bovine Serum Albumin) for 1 h 30-2 h at 37° C. The plate was then washed 3 times with 0.05% PBS-Tween. The samples (fractions containing the proteins of the peptide constructs collected after purification by chromatography on a nickel-agarose column or on a PpL-agarose column) were deposited in duplicate in a final volume of 100 μL/well. After incubation for 1 h 30 at 37° C., 3 washes with PBS-Tween 0.05% were carried out.

Peptide Constructs with the Tag Histidine:

The anti-histidine antibody coupled to peroxidase, diluted 1/1000 in PBS, was added at a rate of 100 μL/well. After 1 h incubation at 37° C. the revelation was carried out by adding 100 μL/well of TMB (3,3′, 5,5′-tetramethylbenzidine) for 5 to 10 min at room temperature and in darkness. The reaction was stopped by adding 50 μL of 2N H₂SO₄ solution. The plate was read at 450 nm by a plate reader.

4.3.4. Indirect Immunofluorescence

The immunofluorescence technique consisted of fixing tachyzoïtes of Toxoplasma gondii on wells and bringing together the peptide constructs to be tested. The binding of the peptide construct to the parasite was revealed by an antibody coupled to a fluorochrome. First, the supernatant of a tachyzoïte culture was recovered and centrifuged (3500 rpm, 15 min) and then the pellet was resuspended with 5 mL of PBS. This step has been executed three times. The third time, the pellet was resuspended so as to obtain 5·10⁶ tachyzoïtes/mL. 20 μL, (i.e 1.105 tachyzoïtes) were deposited on each well, then left to dry under a hood all night. The wells were then stored at −20° C. until use.

Without waiting for the cups to thaw, the tachyzoïtes were fixed in a cold acetone bath for 2 minutes. Each well was then rinsed three times with PBS. Then 30 μL of sample (fractions containing the proteins of the peptide constructs collected after purification by chromatography on a nickel-agarose column or on a PpL-agarose column) were deposited and incubated in a humid chamber at 4° C. for 16 hours.

At the end of the incubation, three washes with PBS were performed before depositing 40 μL of the primary anti-histidine antibody (diluted 1/50) per well. The wells were incubated for 1 h 30 at 37° C. in a humid chamber. After 3 rinses with PBS, the secondary anti-mouse IgG antibody coupled to Alexa488 fluorochrome (dilution 1/500) was added and the wells were incubated in a humid chamber at 37° C. for 1 h 30 min. In order to remove unbound antibodies, 3 washes with PBS were performed. Finally, a blade was then attached to the wells. The observation was carried out under a fluorescence microscope (objective ×40).

5. In Vitro Neutralization Test

HFF (Human Foreskin Fibroblast) line cells were placed in a 96-well plate (P96), at 2·10⁴ cells/well, in 100 DMEM (Dulbecco's Modified Eagle Medium) medium supplemented with 1% glutamine, 1% FCS (Fetal Calf Serum), 1% penicillin-streptomycin, without phenol red, and then incubated for 24 h (37° C., 5% CO₂). Tachyzoïtes, of a RH strain of Toxoplasma gondii transfected with a plasmid encoding β-galactosidase were used. The β-galactosidase gene is under the control of the promoter of the gene encoding the SAG1 protein.

For each well, a volume of 50 μL of DMEM medium containing 100 tachyzoïtes was added to 50 μL of a solution containing from 1.5 to 10 μg of peptide construction to be tested for a pre-incubation of one hour. Then the 100 μl of the pre-incubated solution were placed in the presence of the cells: the peptide constructs and the parasites were therefore added simultaneously.

Alternatively, when this is explicitly mentioned, a volume of 50 μM of DMEM medium containing 100 tachyzoïtes was added to each well and then 50 μL of a solution containing from 1.5 to 10 μg of peptide construction to be tested were placed in each well, presence of cells and parasites without pre-incubation (with a delay of 5 min after the addition of tachyzoïtes).

In both cases, the volume of 50 μL of the solution containing from 1.5 to 10 μg of peptide construction was prepared in the following manner: from a solution containing the peptide construction at different concentrations according to the peptic construction a volume was determined so that this volume contains the desired peptide building mass. This volume was removed and then supplemented with a buffer solution until a volume of 50 μl was obtained.

The plate was incubated for 4 days at 37° C. unless otherwise indicated under 5% CO₂.

In order to analyze the action of each peptide construct, 150 μL of supernatant was removed from each well and 50 μL of lysis buffer (0.1% Triton 100×) was added to each well. The wells were then scraped to lyse all the cells. On a new P96, 50 μL of lysate was deposited in each well, to which 50 μL of a solution of CPRG (Chlorophenol Red-PD-galactopyranoside, 1 μL) in HEPES (100 μL, pH 8). Cleaved by the β-galactosidase present in tachyzoïtes, the CPRG produces a soluble red compound, measurable by spectophotometry. The plate was then incubated at 37° C. for 20 min so that the enzyme could act. Staining was measured at 565 nm.

6. In Vivo Neutralization Test

6.1. Congenital Toxoplasmosis Model

Male and female swiss OF1 mice (January), non-consanguineous albino mice, were used.

For each experiment, the protocol was as follows:
At D1, the setting to the male was carried out by placing 2 females in the presence of a male during 48 h.

The female mice were then monitored daily to determine which pregnant and non-pregnant mice were pregnant.
At D11, corresponding to midgut, the pregnant female mice were all infected by gavage with cysts of the Toxoplasma gondii strain 76k. The mice were then separated into 2 lots. The first batch, called the “infected control”, consisted of mice infected only with T. gondii. The second batch, called “infected and treated”, consisted of infected mice but also treated by the peptide construction. In the latter batch, mice infected with T. gondii received daily, from the day of infection (at mid-gestation) and until parturition, the peptide construct by intraperitoneal injection (15 or 27 μg/mL), mouse/day from a preparation at 120 μg/mL).
After the birth of the young mice, a follow-up of the protection as well as the induced immune response were realized. For this, the young mice were regularly monitored (height/weight) before being sacrificed at the age of 4-5 weeks of life. Their brains were then collected and crushed to evaluate the parasite load, a parameter reflecting the protection.

Moreover, a study of the cellular response was also performed on splenocytes by cytokine assay (interleukin 10 (IL-10), interferon-γ (IFNy)).

6.2. Ocular Toxoplasmosis Model

Female mice, swiss OF1, non-consanguineous albinos, (January), were used and distributed in different batches:
Lot 1: the mice received by intravitreal injection 200 tachyzoïtes of the strain of Toxoplasma gondii ME49 at OJ.
Lot 2: The mice received by intravitreal injection 600 ng of peptide construction (from a preparation at 120 μg/mL) on D0.
Lot 3: the mice were simultaneously injected intravitreally with 200 tachyzoïtes of the ME49 strain and 600 ng of peptide construction (from a preparation at 120 μg/mL) on D0.

The injections, under a volume of 5μí, into the limbus (junction between the cornea and the sclera) were performed under general isoflurane anesthesia using a 250 μl Hamilton syringe (Dutscher) on which was mounted a 32 gauge needle (Dutscher).

The eyes of the mice were then examined under a binocular magnifying glass under general isoflurane anesthesia at D1, D2, D6 and D7 in order to perform the clinical examination. This was performed on the anterior segment (cornea, sclera, conjunctiva, crystalline, aqueous humor) and the posterior segment (vitreous, retina). These clinical examinations were done with a binocular magnifying glass. Corneal hydration was respected to prevent the occurrence of exposure keratitis or corneal edema that would have disrupted the examination.

The eyes examined were divided into five clinical stages:
0: no inflammatory sign,
1: Tyndall in anterior chamber or moderate vitreen,
2: Tyndall in anterior or severe vitreous chamber and/or dilatation of the iris and/or conjunctivo-scleral vessels (posterior subcapsular cataract),
3: corneal disorders and precipitated retrocorneal and/or very severe hyalite,
4: secondary cataract.

### Immunohistochemistry

At D8, the mice were sacrificed. Whole eyes were taken after internal and external canthotomy, removed from the oculomotor muscles and conjunctiva by Vannas scissors and enucleated by traction on the optic nerve. The eyes were frozen at −80° C., directly or after being fixed in 4% paraformaldehyde (one eye per mouse), after inclusion in the embedding medium: oct embadding matrix cellpath. The frozen blocks were cut with cryostat (Leica CM3050) at 10 microns, deposited on a slide (4 cuts per slide) and allowed to dry overnight. Slides can be used directly or frozen at −80° C. and thawed for 1 h. The slides were fixed in four successive acetone baths of 3 min, 60, 70, 80 and 90% then in a 4% paraformaldehyde bath for 3 min and washed twice in PBS for 5 min.

Tagging of tachyzoïtes at the retinal sections was then performed via the use of an infection serum and a secondary antibody (extravidin FITC). 50 of serum of infection were deposited by cutting. The slides rested for 2 hours in a humid chamber at 37° C., then 3 washes were carried out for 3 min with PBS (baths). The secondary antibody was deposited for 30 min: extravidin FITC (sigma E2761) (diluted to 1/1000) (labeling of tachyzoïtes in green). The three washes for 3 min in PBS at room temperature were renewed and the counterstaining was done with Hoescht (Molecular Probes 1333342 H3573) diluted in water at 1/2000 (staining of the nuclei in blue) for a minute. 50 μL per cut have been deposited. The slides were then washed 3 times for 3 min at room temperature (baths) and mounted with Vectashield H1000 without leaving bubbles. After drying, the slides were observed under a fluorescence microscope.

### Example 2 Production and Analysis of Proteins of Peptide Construction SG0

### 1. Construction of the Expression Vector

The gene coding for the SG0 peptide construct, represented by the sequence SEQ ID NO: 10, was obtained by synthesis from the company Thermo Fisher Scientific GENEART, cloned into a vector. The nucleotide sequences encoding the variable domains of the heavy (VH) and light (VL) chains were optimized for production of the protein in a prokaryotic system.

The vector containing the gene encoding the SG0 peptide construct was then digested with a pair of endonucleases (Table I). The DNA fragment resulting from the double digestion was then isolated by agarose gel electrophoresis and then purified. This DNA fragment encoding the peptide construct (insert) was then inserted into the expression vector pET22b, previously digested with the same pair of endonucleases (Table I) via a ligation. The ligation products were used to transform Escherichia coli BL21 bacteria.

### 2. Production, Purification and Protein Analysis

The proteins of the SGI peptide construct were produced in a bacterial system (Escherichia coli BL21). The proteins produced were extracted from the periplasm by osmotic shock.

The proteins were then purified by affinity chromatography on a nickel-agarose column.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 3 Production and Analysis of Peptides of Peptide Construction SG5

### 1. Construction of the Expression Vector

The nucleotide sequence of SG5 represented by the sequence SEQ ID NO: 11 was obtained using a PCR technique.

First, the nucleotide sequence of the variable domain of the light chain (VL domain) of the SG0 peptide construct was amplified by PCR. The PCR product was then purified and cloned into the pGEMT vector.

TG1 bacteria were transformed with the pGEMT vector. After culturing the bacteria, the plasmid was purified using the Plasmid Minikit I kit. The plasmid was then digested with BamHI and XhoI. The DNA fragment resulting from the double digestion was then isolated by agarose gel electrophoresis and then purified. This DNA fragment, coding the variable domain of the light chain of the peptide construct SG0 (insert), was then inserted into the expression vector pET22b, previously digested with the same pair of endonucleases, via a ligation.

BL21 bacteria made competent were then transformed with the ligation product.

### 2. Production, Purification and Protein Analysis

The proteins of the SG5 peptide construct were produced in a bacterial system (Escherichia coli BL21). The proteins produced were extracted from the periplasm by osmotic shock.

The proteins were then purified by affinity chromatography on a nickel-agarose column. The purified proteins were then analyzed by FPLC (structural analysis) and then by polyacrylamide gel electrophoresis (SDS-PAGE) under denaturing conditions. The proteins were then characterized by immuno-detection using a Western blot.

### Example 4 Production and Analysis of Proteins of the Peptide Construction SG2-LH

### 1. Construction of the Expression Vector

The gene coding for the SG2-LH peptide construct represented by the sequence SEQ ID NO: 27, was obtained by synthesis from the company Thermo Fisher Scientific GENEART, cloned into a vector. The nucleotide sequences encoding the variable domains of the heavy (VH) and light (VL) chains were optimized for production of the protein in the eukaryotic system.

The vector containing the gene encoding the SG2-LH peptide construct was then digested with a pair of endonucleases (Table 1). The DNA fragment resulting from the double digestion was then isolated by agarose gel electrophoresis and then purified. This DNA fragment encoding the peptide construct (insert) was then inserted into the pMT-Bip expression vector, previously digested with the same pair of endonucleases (Table I) via a ligation. The ligation products were used to transform Escherichia coli DH5 bacteria, in order to obtain after culture and purification (maxi prep) a sufficiently large amount of plasmid to perform the transfection of S2 cells.

### 2. Production, Purification and Protein Analysis

The proteins of the peptide construct SG2-LH were produced in eukaryotic system. The proteins produced were secreted into the culture medium of S2 cells after induction with copper sulphate.

The proteins were then purified by affinity column chromatography on PpL agarose.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 5: Production and Analysis of Peptide Construction Proteins DbSG2

### 1. Construction of the Expression Vector

The gene coding for the DbSG2 peptide construct represented by the sequence SEQ ID NO: 13 was obtained by synthesis from the company Thermo Fisher Scientific GENEART, cloned into a vector. The nucleotide sequences encoding the variable domains of the heavy (VH) and light (VL) chains were optimized for production of the protein in the eukaryotic system.

The vector containing the gene encoding the peptide construct DbSG2 was then digested with a pair of endonucleases (Table I). The DNA fragment resulting from the double digestion was then isolated by agarose gel electrophoresis and then purified. This DNA fragment encoding the peptide construct (insert) was then inserted into the pMT-Bip expression vector, previously digested with the same pair of endonucleases (Table 1) via a ligation. The ligation products were used to transform Escherichia coli DH5 bacteria, in order to obtain after culture and purification (maxi prep) a sufficiently large amount of plasmid to perform the transfection of S2 cells.

### 2. Production, Purification and Protein Analysis

The proteins of the peptide construct DbSG2 have been produced in the eukaryotic system. The proteins produced were secreted into the culture medium of S2 cells after induction with copper sulphate.

The proteins were then purified by affinity column chromatography on PpL agarose.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 6: Production and Analysis of Proteins of the Peptide Construction SG2-CH3

### 1. Construction of the Expression Vector

The gene coding for the SG2-CH3 peptide construct represented by the sequence SEQ ID NO: 14, was obtained by synthesis from the company Thermo Fisher Scientific GENEART, cloned into a vector. The nucleotide sequences encoding the variable domains of the heavy (VH) and light (VL) chains were optimized for production of the protein in the eukaryotic system.

The vector containing the gene encoding the SG2-CH3 peptide construct was then digested with a pair of endonucleases (Table I). The DNA fragment resulting from the double digestion was then isolated by agarose gel electrophoresis and then purified. This DNA fragment encoding the peptide construct (insert) was then inserted into the pMT-Bip expression vector, previously digested with the same pair of endonucleases (Table 1) via a ligation. The ligation products were used to transform Escherichia coli DH5a bacteria, in order to obtain after culture and purification (maxi prep) a sufficiently large amount of plasmid to perform the transfection of S2 cells.

### 2. Production, Purification and Protein Analysis

The proteins of the SG2-CH3 peptide construct were produced in the eukaryotic system. The proteins produced were secreted into the culture medium of S2 cells after induction with copper sulphate.

The proteins were then purified by affinity column chromatography on PpL agarose.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 7 Production and Analysis of Proteins of the SG2-Fc2a Peptide Construction

### 1. Construction of the Expression Vector

The gene coding for the SG2-Fc2a peptide construct represented by the sequence SEQ ID NO: 16 was obtained by synthesis from the company Thermo Fisher Scientific GENEART, cloned into a vector. The nucleotide sequences encoding the variable domains of the heavy (VH) and light (VL) chains were optimized for production of the protein in the eukaryotic system.

The vector containing the gene encoding the SG2-Fc2a peptide construct was then digested with a pair of endonucleases (Table I). The DNA fragment resulting from the double digestion was then isolated by agarose gel electrophoresis and then purified. This DNA fragment encoding the peptide construct (insert) was then inserted into the pMT-Bip expression vector, previously digested with the same pair of endonucleases (Table I) via a ligation. The ligation products were used to transform Escherichia coli DH5 bacteria, in order to obtain after culture and purification (maxi prep) a sufficiently large amount of plasmid to perform the transfection of S2 cells.

### 2. Production, Purification and Protein Analysis

The proteins of the SG2-Fc2a peptide construct were produced in the eukaryotic system. The proteins produced were secreted into the culture medium of S2 cells after induction with copper sulphate.

The proteins were then purified by affinity column chromatography on PpL agarose. The purified proteins were then analyzed by FPLC (structural analysis) and then by polyacrylamide gel electrophoresis (SDS-PAGE) under denaturing conditions. The proteins were then characterized by immuno-detection using a Western blot.

### Example 8: Production and Analysis of Proteins of the Peptide Construction SG2-HL

### 1. Production in Eukaryotic System

### 1.1. Construction of the Expression Vector

The gene coding for the SG2-HL peptide construct represented by the sequence SEQ ID NO: 12, was obtained by synthesis from the company Thermo Fisher Scientific GENEART, cloned into a vector. The nucleotide sequences encoding the variable domains of the heavy (VH) and light (VL) chains were optimized for production of the protein in the eukaryotic system.

The vector containing the gene encoding the SG2-HL peptide construct was then digested with a pair of endonucleases (Table I). The DNA fragment resulting from the double digestion was then isolated by agarose gel electrophoresis and then purified. This DNA fragment encoding the peptide construct (insert) was then inserted into the pMT-Bip expression vector, previously digested with the same pair of endonucleases (Table I) via a ligation. The ligation products were used to transform Escherichia coli DH5 bacteria, in order to obtain after culture and purification (maxi prep) a sufficiently large amount of plasmid to perform the transfection of S2 cells.

### 1.2. Production, Purification and Protein Analysis

The proteins of the SG2-HL peptide construct were produced in the eukaryotic system. The proteins produced were secreted into the culture medium of S2 cells after induction with copper sulphate. The proteins were then purified by affinity column chromatography on PpL agarose.

The purified proteins were then analyzed by FPLC (structural analysis) and then by polyacrylamide gel electrophoresis (SDS-PAGE) under denaturing conditions. The proteins were then characterized by immuno-detection using a Western blot.

### 2. Production in Prokaryotic System

### 2.1. Construction of the Expression Vector

The gene coding for the SG2-HL peptide construct represented by the sequence SEQ ID NO: 12, was obtained by synthesis from the company Thermo Fisher Scientific GENEART, cloned into a vector. The nucleotide sequences encoding the variable domains of the heavy (VH) and light (VL) chains were optimized for production of the protein in a prokaryotic system.

The vector containing the gene encoding the SG2-HL peptide construct was then digested with a pair of endonucleases (Table I). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table I) via a ligation. The ligation products were used to transform Escherichia coli HB2151 bacteria.

### 2.2. Production, Purification and Protein Analysis

The proteins of the SG2-HL peptide construct were produced in a prokaryotic system (Escherichia coli HB2151). The proteins produced were extracted from the periplasm by osmotic shock.

The proteins were then purified by affinity chromatography on a PpL-agarose column.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 9 Production and Analysis of Peptide Construction Proteins DbSG2-CH3

### 1. Construction of the Expression Vector

The gene encoding the peptide construct DbSG2-CH3 represented by the sequence SEQ ID NO: 15, was obtained by synthesis from the company Thermo Fisher Scientific GENEART, cloned into a vector. The nucleotide sequences encoding the variable domains of the heavy (VH) and light (VL) chains were optimized for production of the protein in the eukaryotic system.

The vector containing the gene encoding the peptide construct DbSG2-CH3 was then digested using a pair of endonucleases (Table 1). The DNA fragment resulting from the double digestion was then isolated by agarose gel electrophoresis and then purified. This DNA fragment encoding the peptide construct (insert) was then inserted into the pMT-Bip expression vector, previously digested with the same pair of endonucleases (Table I) via a ligation. The ligation products were used to transform Escherichia coli DH5a bacteria, in order to obtain after culture and purification (maxi prep) a sufficiently large amount of plasmid to perform the transfection of S2 cells.

### 2. Production, Purification and Protein Analysis

The proteins of the peptide construction DbSG2-CH3 were produced in eukaryotic system. The proteins produced were secreted into the culture medium of S2 cells after induction with copper sulphate.

The proteins were then purified by affinity column chromatography on PpL agarose.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 10: Production and Analysis of Proteins of the Peptide Construct scFvSG1

### 1. Construction of the Expression Vector

The gene coding for the scFvSG1 peptide construct represented by the sequence SEQ ID NO: 17, was obtained by synthesis from the company Thermo Fisher Scientific GENEART, cloned in a vector. The nucleotide sequences encoding the variable domains of the heavy (VH) and light (VL) chains were optimized for production of the protein in a prokaryotic system. The vector containing the gene coding for the scFvSG1 peptide construct was then digested with a pair of endonucleases (Table I). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table I) via a ligation. The ligation products were used to transform Escherichia coli HB2151 bacteria.

### 2. Production, Purification and Protein Analysis

The proteins of the peptide construct scFvSG1 were produced in prokaryotic system (Escherichia coli HB21 1). The proteins produced were extracted from the periplasm by osmotic shock.

The proteins were then purified by affinity chromatography on a PpL-agarose column.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 11: Production and Analysis of Proteins of Peptide Construction DbF3S2

### 1. Construction of the Expression Vector

The gene encoding the peptide construct DbF3S2 represented by the sequence SEQ ID NO: 18, was obtained by synthesis from the company Thermo Fisher Scientific GENEART, cloned into a vector. The nucleotide sequences encoding the variable domains of the heavy (VH) and light (VL) chains were optimized for production of the protein in a prokaryotic system.

The vector containing the gene coding for the peptide construct DbF3S2 was then digested using a pair of endonucleases (Table 1). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table I) via a ligation. The ligation products were used to transform Escherichia coli HB2151 bacteria.

### 2. Production, Purification and Protein Analysis

The proteins of the peptide construct DbF3S2 were produced in prokaryotic system (Escherichia coli HB2151). The proteins produced were extracted from the periplasm by osmotic shock.

The proteins were then purified by affinity chromatography on a PpL-agarose column.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 12: Production and Analysis of Peptide Construction Proteins DbF4S2

### 1. Construction of the Expression Vector

The gene encoding the peptide construct DbF4S2 represented by the sequence SEQ ID NO: 19, was obtained by synthesis from the company Thermo Fisher Scientific GENEART, cloned into a vector. The nucleotide sequences encoding the variable domains of the heavy (VH) and light (VL) chains were optimized for production of the protein in a prokaryotic system.

The vector containing the gene encoding the peptide construct DbF4S2 was then digested using a pair of endonucleases (Table I). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table I) via a ligation. The ligation products were used to transform Escherichia coli HB2151 bacteria.

### 2. Production, Purification and Protein Analysis

The proteins of the peptide construction DbF4S2 were produced in prokaryotic system (Escherichia coli HB2151). The proteins produced were extracted from the periplasm by osmotic shock.

The proteins were then purified by affinity chromatography on a PpL-agarose column. The purified proteins were then analyzed by FPLC (structural analysis) and then by polyacrylamide gel electrophoresis (SDS-PAGE) under denaturing conditions. The proteins were then characterized by immuno-detection using a Western blot.

### Example 13: Production and Analysis of Peptide Construction Proteins scFvF5S2

### 1. Construction of the Expression Vector

The gene coding for the scFvF5S2 peptide construct represented by the sequence SEQ ID NO: 20, was obtained by synthesis from the company Thermo Fisher Scientific GENEART, cloned into a vector. The nucleotide sequences encoding the variable domains of the heavy (VH) and light (VL) chains were optimized for production of the protein in a prokaryotic system.

The vector containing the gene coding for the scFvF5S2 peptide construct was then digested with a pair of endonucleases (Table 1). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table 1) via a ligation. The ligation products were used to transform Escherichia coli HB2151 bacteria.

### 2. Production, Purification and Protein Analysis

The proteins of the peptide construct scFvF5S2 were produced in a prokaryotic system (Escherichia coli HB21 1). The proteins produced were extracted from the periplasm by osmotic shock.

The proteins were then purified by affinity chromatography on a Ppl-agarose column.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 14: Production and Analysis of Peptide Construction Proteins scFvF5S4

### 1. Construction of the Expression Vector

The gene coding for the scFvF5S4 peptide construct represented by the sequence SEQ ID NO: 21, was obtained by synthesis from the company Thermo Fisher Scientifique GENEA T, cloned into a vector. The nucleotide sequences encoding the variable domains of the heavy (VH) and light (VL) chains were optimized for production of the protein in a prokaryotic system.

The vector containing the gene coding for the scFvF5S4 peptide construct was then digested with a pair of endonucleases (Table I). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table I) via a ligation. The ligation products were used to transform Escherichia coli HB2151 bacteria.

### 2. Production, Purification and Protein Analysis

The proteins of the peptide construct scFvF5S4 were produced in prokaryotic system (Escherichia coli HB21 1). The proteins produced were extracted from the periplasm by osmotic shock.

The proteins were then purified by affinity chromatography on a PpL-agarose column. The purified proteins were then analyzed by FPLC (structural analysis) and then by polyacrylamide gel electrophoresis (SDS-PAGE) under denaturing conditions. The proteins were then characterized by immuno-detection using a Western blot.

### Example 15: Production and Analysis of Proteins of the Peptide Construct scFvF5S5

### 1. Construction of the Expression Vector

The gene coding for the scFvF5S5 peptide construct represented by the sequence SEQ ID NO: 22, was obtained by synthesis from the company Thermo Fisher Scientific GENEART, cloned into a vector. The nucleotide sequences encoding the variable domains of the heavy (VH) and light (VL) chains were optimized for production of the protein in a prokaryotic system. The vector containing the gene coding for the scFvF5S5 peptide construct was then digested with a pair of endonucleases (Table I). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table 1) via a ligation. The ligation products were used to transform Escherichia coli HB2151 bacteria.

### 2. Production, Purification and Protein Analysis

The proteins of the peptide construct scFvF5S5 were produced in prokaryotic system (Escherichia coli HB21 1). The proteins produced were extracted from the periplasm by osmotic shock.

The proteins were then purified by affinity chromatography on a Ppl-agarose column.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 16: Production and Analysis of Proteins of the Peptide Construct scFvF5S6

### 1. Construction of the Expression Vector

The gene coding for the scFvF5S6 peptide construct represented by the sequence SEQ ID NO: 23, was obtained by synthesis from the company Thermo Fisher Scientific GENEART, cloned in a vector. The nucleotide sequences encoding the variable domains of the heavy (VH) and light (VL) chains were optimized for production of the protein in a prokaryotic system.

The vector containing the gene coding for the scFvF5S6 peptide construct was then digested with a pair of endonucleases (Table I). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table I) via a ligation. The ligation products were used to transform Escherichia coli HB2151 bacteria.

### 2. Production, Purification and Protein Analysis

The proteins of the peptide construct scFvF5S6 were produced in a prokaryotic system (Escherichia coli HB2151). The proteins produced were extracted from the periplasm by osmotic shock.

The proteins were then purified by affinity chromatography on a PpL-agarose column.

The purified proteins were then analyzed by FPLC (structural analysis) and then by polyacrylamide gel electrophoresis (SDS-PAGE) under denaturing conditions. The proteins were then characterized by immuno-detection using a Western blot.

### Example 17: Production and Analysis of Hum-scFvH2S Peptide Constructs

### 1. Construction of the Expression Vector

The gene coding for the Hum-scFvH2S peptide construct represented by the sequence SEQ ID NO: 24, was obtained by synthesis from the company Thermo Fisher Scientific GENEART, cloned into a vector. The nucleotide sequences encoding the variable domains of the heavy (VH) and light (VL) chains were optimized for production of the protein in a prokaryotic system.

The vector containing the gene coding for the Hum-scFvH2S peptide construct was then digested using a pair of endonucleases (Table I). The DNA fragment resulting from the double digestion was then isolated by agarose gel electrophoresis and then purified. This DNA fragment encoding the peptide construct (insert) was then inserted into the expression vector pSW1, previously digested with the same pair of endonucleases (Table I) via a ligation. The ligation products were used to transform Escherichia coli HB2151 bacteria.

### 2. Production, Purification and Protein Analysis

The proteins of the Hum-scFvH2S peptide construct were produced in a prokaryotic system (Escherichia coli HB2151). The proteins produced were extracted from the periplasm by osmotic shock.

The proteins were then purified by affinity chromatography on a PpL-agarose column. The purified proteins were then analyzed by FPLC (structural analysis) and then by polyacrylamide gel electrophoresis (SDS-PAGE) under denaturing conditions. The proteins were then characterized by immuno-detection using a Western blot.

### Example 18 Analysis of the Fixation of the Peptide Construction SG0 on Toxoplasma gondii

The fixation of the SG0 peptide construct on Toxoplasma gondii is observed by immunofluorescence, according to the protocol described in paragraph 4.3.4 of Example 1 (Materials and Methods).

Tachyzoïtes of Toxoplasma gondii were fixed on cups at the rate of 1·10⁵ tachyzoïtes per well. The sample containing the SG0 peptide construct was plated on the wells which were incubated in a humid chamber at 4° C. overnight.

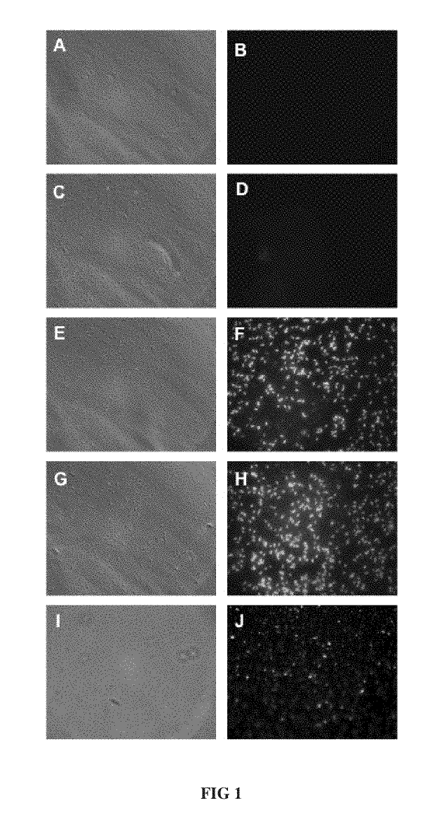

The results are shown in FIG. 1.

The presence of fluorescence in part F of FIG. 1 shows that the SG0 binds to Toxoplasma gondii tachyzoïtes. SG0 fixation is also visible in white light in part E of FIG. 1.

### Example 19: Analysis of the Fixation of the SG5 Peptide Construct on Toxoplasma gondii

The fixation of the SG5 peptide construct on Toxoplasma gondii is observed by immunofluorescence, according to the protocol described in paragraph 4.3.4 of Example 1 (Materials and Methods). Tachyzoïtes of Toxoplasma gondii were fixed on cups at the rate of 1·10⁵ tachyzoïtes per well.

The sample containing the SG5 peptide construct was plated on the wells which were incubated in a humid chamber at 4° C. overnight.

The results are shown in FIG. 1.

The presence of fluorescence in part H of FIG. 1 shows that SG5 binds to the tachyzoïtes of Toxoplasma gondii. The fixation of SG5 is also visible in white light in part G of FIG. 1.

### Example 20: Analysis of the Fixation of the Peptide Construction DbF3S2 on Toxoplasma gondii

The fixation of the DbF3S2 peptide construct on Toxoplasma gondii is observed by immunofluorescence, according to the protocol described in paragraph 4.3.4 of Example 1 (Materials and Methods). Tachyzoïtes of Toxoplasma gondii were fixed on cups at the rate of 1·10⁵ tachyzoïtes per well.

The sample containing the peptide construct DbF3S2 was plated on the wells which were incubated in a humid chamber at 4° C. overnight.

The results are shown in FIG. 1.

The presence of fluorescence in part J of FIG. 1 shows that DbF3S2 binds to Toxoplasma gondii tachyzoïtes. The fixation of SG5 is also visible in white light in part I of FIG. 1.

### Example 21: Analysis of the Fixation of the Peptide Construction scFvF5S2 on Toxoplasma gondii

The fixation of the scFvF5S2 peptide construct on Toxoplasma gondii is observed by immunofluorescence, according to the protocol described in paragraph 4.3.4 of Example 1 (Materials and Methods). Tachyzoïtes of Toxoplasma gondii were fixed on cups at the rate of 1.10 tachyzoïtes per well.

The sample containing the scFvF5S2 peptide construct was plated on the wells which were incubated in a humid chamber at 4° C. overnight.

### Example 22: Analysis of the Fixation of the Peptide Construction scFvF5S4 on Toxoplasma gondii

The attachment of the peptide construct scFvF5S4 to Toxoplasma gondii is observed by immunofluorescence, according to the protocol described in paragraph 4.3.4 of Example 1 (Materials and Methods). Tachyzoïtes of Toxoplasma gondii were fixed on cups at the rate of 1·10⁵ tachyzoïtes per well.

The sample containing the scFvF5S4 peptide construct was plated on the wells which were incubated in a humid chamber at 4° C. overnight.

### Example 23: Analysis of the Fixation of the Peptide Construction scFvF5S5 on Toxoplasma gondii

The fixation of the scFvF5S5 peptide construct on Toxoplasma gondii is observed by immunofluorescence, according to the protocol described in paragraph 4.3.4 of Example 1 (Materials and Methods).

Tachyzoïtes of Toxoplasma gondii were fixed on cups at the rate of 1.10 tachyzoïtes per well. The sample containing the peptide construct scFvF5S5 was plated on the wells which were incubated in a humid chamber at 4° C. overnight.

### Example 24: Analysis of the Fixation of the Peptide Construction scFvF5S6 on Toxoplasma gondii

The fixation of the peptide construct scFvF5S6 on Toxoplasma gondii is observed by immunofluorescence, according to the protocol described in paragraph 4.3.4 of Example 1 (Materials and Methods).

Tachyzoïtes of Toxoplasma gondii were fixed on cups at the rate of 1·10⁵ tachyzoïtes per well. The sample containing the scFvF5S6 peptide construct was plated on the wells which were incubated in a humid chamber at 4° C. overnight.

### Example 25: Efficacy of the SG0 Construct on the In Vitro Neutralization of HFF Cell Invasion by Toxoplasma gondii Tachyzoïtes

HFF line cells were plated in a 96-well plate (P96) at 2·10⁴ cells/well. For each well, a certain volume of a solution of SG0 at 109 μg mL was taken so that it contained 1.5 μg, 1.6 μg, 3 μg, 6 μg or 10 μg of SG0 then this last was completed with a buffer solution until a volume of 50 μl.

After 24 h incubation of the HFF cells in the wells, the 50 μl of the solution containing the peptide construct SG0 at different concentrations, so that the amounts of SG0 are 1.5 μg, 1.6 μg, 3 μg, 6 μg or 10 μg, were added to each well, either simultaneously with the addition of 100, 1000 or 10,000 tachyzoïtes, of a RH strain of Toxoplasma gondii, transfected with a plasmid encoding β-galactosidase, after a preincubation of one hour, or with a delay of 5 min after the addition of tachyzoïtes (without preincubation). The plate was incubated for 4 days at 37° C., or at room temperature, under 5% CO2.

The in vitro neutralization of HFF cell invasion by Toxoplasma gondii tachyzoïtes is evaluated by measuring the optical density (OD) at the 565 nm wavelength.

The results are presented in FIGS. 2A, 2B, 2C, 2D and in FIG. 3.

The SG0 peptide construct inhibits the cellular invasion of HFF cells by Toxoplasma gondii tachyzoïtes compared to the irrelevant B6P antibody regardless of the amount of tachyzoïtes (100, 1000 or 10.000) (FIGS. 2A and 2B).

The inhibition effect persists that one is in physiological condition (37° C.) or at ambient temperature (FIG. 2 C).

Pre-incubation is not a prerequisite for the inhibitory power of SG0, since without pre-incubation. SG0 is able to inhibit cell invasion as efficiently as pre-incubation (FIG. 2 D).

### Example 26: Efficacy of SG5 Construction on the In Vitro Neutralization of HFF Cell Invasion by Toxoplasma gondii Tachyzoïtes

HFF line cells were plated in a 96-well plate (P96) at 2·10⁴ cells/well.

For each well, a certain volume of a solution of SG5 at 147 μg/mL was taken so that it contained 1.5 μg, 3 μg or 6 g of SG5, then the latter was supplemented with a solution buffer until a volume of 50 μL is obtained. After 24 h incubation of the HFF cells in the wells, the 50 of the solution containing the SG5 peptide construct at different concentrations, so that the amounts of SG5 are 1.5 μg, 3 μg or 6 μg, have in each well, simultaneously with 100 tachyzoïtes, were added a RH strain of Toxoplasma gondii, transfected with a plasmid encoding β-galactosidase. The plate was incubated for 4 days at 37° C. under 5% CO 2.

The in vitro neutralization of HFF cell invasion by Toxoplasma gondii tachyzoïtes is evaluated by measuring the optical density (OD) at the 565 nm wavelength.

The results are shown in FIG. 3.

SG5 is able to inhibit cellular invasion by the parasite. In addition, a dose-dependent effect is observed.

### Example 27: Efficacy of the DbF3S2 Construct on the In Vitro Neutralization of HFF Cell Invasion by Toxoplasma gondii Tachyzoïtes

HFF line cells were plated in a 96-well plate (P96) at 2·10⁴ cells/well.

For each well, a certain volume of a solution of DbF3S2 at 155 μg/mL was taken in such a way that it contained 1, 5 μg or 3 μg of DbF3S2 and the latter was then supplemented with a buffer solution up to to obtain a volume of 50 μi.

After 24 hours of incubation of the HFF cells in the wells, the 50 μL of the solution containing the peptide construct DbF3S2 at different concentrations, so that the amounts of DbF3S2 are 1, 5 μg or 3 μg, were added, in each well, simultaneously with 100 tachyzoïtes, of a RH strain of Toxoplasma gondii, transfected with a plasmid encoding J-galactosidase. The plate was incubated for 4 days at 37° C. under 5% CO₂.

The in vitro neutralization of HFF cell invasion by Toxoplasma gondii tachyzoïtes is evaluated by measuring the optical density (OD) at the 565 nm wavelength.

The results are shown in FIG. 3. DbF3S2 is able to inhibit cellular invasion by the parasite, but it seems less effective than SG0 and SG5. No dose-dependent effect is observed for this peptide construct.

### Example 28: Efficacy of the Peptide Construction SG0 in the Treatment of Toxoplasmosis in a Mouse Model of Congenital Toxoplasmosis

Male and female swiss OF1 mice (January), non-consanguineous albino mice, were used.

Three experiments were performed, including parameters (number of mice per lot (mouse number/lot), number of Toxoplasma gondii strain 76K cysts administered (# of cysts (76K)) and amount of SG0 administered daily to treated mice. (Quantity of SG0 in μg)) are summarized in Table II.

TABLE II

| | EXPERIENCE 1 | | EXPERIENCE 2 | | EXPERIENCE 3 | |
|---|---|---|---|---|---|---|
| | Infected | Infected & | Infected | Infected & | Infected | Infected & |
| | Witness | treated | Witness | treated | Witness | treated |
| Number mice/lot | 6 | 7 | 2 | 2 | 3 | 3 |
| No cystes (76K) | 10 | 10 | 30 | 30 | 30 | 30 |
| Quantity of SG0 (μg) | — | 15 | — | 27 | — | 15 |

The results of experiment 1 are shown in FIG. 4. The mice of the “infected and treated” batch of mice have a greater weight than the mice of the “infected control” group.

### Example 29: Efficacy of SG0 Peptide Construction in the Treatment of Toxoplasmosis in a Mouse Model of Ocular Toxoplasmosis

Female OF1 (January) swiss mice, non-consanguineous albinos, were used.

The eyes of the mice were examined under a binocular magnifying glass, under general isoflurane anesthesia, on D1, D2, D6 and D7 in order to perform the clinical examination on the anterior segment (cornea, sclera, conjunctiva, crystalline, aqueous humor) and the posterior segment (vitreous, retina).

At day 8, the mice were sacrificed, the eyes removed and frozen. Sections of the eyes were made and placed on a slide (4 cuts per slide) and allowed to dry overnight. The slides were observed under a fluorescence microscope.

The results are shown in FIG. 5.

The labeling carried out with the infection serum seems to highlight the presence of tachyzoïtes in the retina of the mice injected intravitreously with parasites alone (line ME49, box 3 of FIG. 5) whereas no tachyzoïtes were found in the retina of the mice receiving the tachyzoïtes and the SG0 or SG0 alone (respectively line SG0+ME49 box 3 and line SG0 box 3 of FIG. 5).

These results indicate that peptide construction SG0 limits parasite proliferation.

### Example 30: Efficacy of SG0, SG2-HL, SG2-LH, DbSG2, SG2-Fc2a and SG2-CH3 Constructs on the In Vitro Neutralization of HFF Cell Invasion by Toxoplasma gondii Tachyzoïtes

HFF line cells were plated in a 96-well plate (P96) at 2·10⁴ cells/well.

For each well, a certain volume of a 125 μg/mL solution of SG0, 75 μg/mL SG2-HL, 90 μg/mL SG2-LH, 80 μg mL DbSG2, SG2-Fc2a at 85 μg/mL, or of SG2-CH3 at 75 μg/mL was taken so that it contains 5 μg of SiO, 5 μg of SG2-HL, 5 μg of SG2-LH, 5 μg of DbSG2, 5 μg of SG2-Fc2a or 5 μg of SG2-CH3, then the latter was supplemented with a buffer solution until a volume of 50 μl.

After 24 h incubation of the HFF cells in the wells, the 50 μL of the solution containing the peptide construct SG0, SG2-HL, SG2-LH, DbSG2, SG2-Fc2a or SG2-CH3, so that the amounts of 5 μg constructs were added in each well, simultaneously with 1000 tachyzoïtes, of a RH strain of Toxoplasma gondii, transfected with a plasmid encoding β-galactosidase. The plate was incubated for 4 days at 37° C. under 5% CO 2.

The in vitro neutralization of HFF cell invasion by Toxoplasma gondii tachyzoïtes is evaluated by measuring the optical density (OD) at the 565 nm wavelength.

The results are shown in FIG. 6.

SG0 is able to inhibit cellular invasion by the parasite.

SG2-HL is able to inhibit cellular invasion by the parasite.

SG2-LH is able to inhibit cellular invasion by the parasite.

DbSG2 is able to inhibit cellular invasion by the parasite.

SG2-Fc2a is able to inhibit cellular invasion by the parasite.

SG2-CH3 is able to inhibit cellular invasion by the parasite. Example 31; Analysis of the fixation of the peptide construct DbSG2, SG2-HL,

SG2-LH. SG2-Fc2a and SG2-CH3 on Toxoplasma gondii

The fixation of the peptide constructs DbSG2. SG2-HL. SG2-LH, SG2-Fc2a and SG2-CH3 on Toxoplasma gondii is observed by immunofluorescence, according to the protocol described in paragraph 4.3.4 of Example 1 (Materials and Methods). Tachyzoïtes of Toxoplasma gondii were fixed on cups at the rate of 1·10⁵ tachyzoïtes per well.

The sample containing the peptide construct DbSG2, SG2-HL, SG2-LH, SG2-Fc2a or SG2-CH3 was plated on the wells which were incubated in a humid chamber at 4° C. overnight.

The results are shown in FIG. 7.

The presence of fluorescence in parts B to F of FIG. 7 shows that the peptide construct DbSG2, SG2-HL, SG2-LH, SG2-Fc2a or SG2-CH3 binds to Toxoplasma gondii tachyzoïtes.

1. A method for treating toxoplasmosis, including ocular toxoplasmosis, congenital toxoplasmosis, and behavioral disorders related to the presence of Toxoplasma gondii, the method comprising administering a peptide construct not containing a CH1 region, recognizing the SAG1 antigen of Toxoplasma gondii and capable of neutralizing Toxoplasma gondii invasion of cells.

2. The method of claim 1, wherein the peptide construct comprises the variable regions of the heavy chain and the light chain of a first antibody recognizing the SAG1 antigen of Toxoplasma gondii, in particular the monoclonal antibody 4F11E12, and which may contain all or part of the constant region lacking a CH1 region, the heavy chain of a second antibody, in particular a murine IgG2a immunoglobulin, said peptide construct recognizing the SAG1 antigen of Toxoplasma gondii and being capable of neutralizing the invasion of the cells by Toxoplasma gondii.

3. The method of claim 1, wherein the peptide construct recognizes the conformational epitope formed by the amino acids at positions 35 to 37, 39, 41, 42, 45, 48, 50, 59 to 65 and 112 to 114 of the amino acid sequence SEQ ID NO: 3.

4. The method of claim 1, wherein the peptide construct comprises the following six CDRs:
a CDR1 having at least 95%, at least 96%, at least 97%, at least 98% or at least 99% identity with the sequence SEQ ID NO: 4, or consisting of the amino acid sequence SEQ ID NO: 4,
a CDR2 having at least 95%, at least 96%, at least 97%, at least 98% or at least 99% % identity with the sequence SEQ ID NO: 5, or consisting of the amino acid sequence SEQ ID NO: 5,
a CDR3 having at least 95%, at least 96%, at least 97%, at least 98% % or at least 99% identity with the sequence SEQ ID NO: 6, or consisting of the amino acid sequence SEQ ID NO: 6,
a CDR4 having at least 95%, at least 96%, at least 97%, at least 98% or at least 99% identity with the sequence SEQ ID NO: 7, or consisting of the amino acid sequence SEQ ID NO: 7,
a CDR5 having at least 95%, at least 96%, at least 97%, at least 98% or at least 99% identity with the sequence SEQ ID NO: 8, or consisting of the amino acid sequence SEQ ID NO: 8, and
a CDR6 having at least 95%, at least 96%, at least 97%, at least 98% or at least 99% identity with the sequence SEQ ID NO: 9, or consisting of the amino acid sequence SEQ ID NO: 9,
provided that said peptide construct retains its ability to neutralize the invasion of cells by Toxoplasma gondii.

5. The method of claim 1, devoid of the CH2 and CH3 regions of the aforementioned second antibody, or comprising a CH3 region and devoid of CH2 region of the aforementioned second antibody, or comprising a CH2 region and a CH3 region of the aforementioned second antibody.

6. The method of claim 1, wherein the peptide construct is chosen from:
scFv, in particular a scFv consisting of the amino acid sequence SEQ ID NO: 10, a scFv consisting of the amino acid sequence SEQ ID NO: 12, a scFv consisting of the amino acid sequence SEQ ID NO: 17, a scFv consisting of the amino acid sequence SEQ ID NO: 20, a scFv consisting of the amino acid sequence SEQ ID NO: 21, a scFv consisting of the amino acid sequence SEQ ID NO: 22, a scFv consisting of the amino acid sequence SEQ ID NO: 23, a scFv consisting of the amino acid sequence SEQ ID NO: 24, a scFv consisting of the amino acid sequence SEQ ID NO: 25, a scFv consisting of the amino acid sequence SEQ ID NO: 27, a scFv consisting of the amino acid sequence SEQ ID NO: 32, a scFv consisting of the amino acid sequence SEQ ID NO: 35, a scFv consisting of the amino acid sequence SEQ ID NO: 36, a scFv constit SEQ ID NO: 37, a scFv consisting of the amino acid sequence SEQ ID NO: 38 or a scFv consisting of the amino acid sequence SEQ ID NO: 39,
a diabody, in particular a diabody consisting of two amino acid sequences of sequence SEQ ID NO: 11, a diabody consisting of two amino acid sequences of sequence SEQ ID NO: 13, a diabody consisting of two amino acid sequences of sequence SEQ ID NO: 18, a diabody consisting of two amino acid sequences of sequence SEQ ID NO: 19, a diabody consisting of two amino acid sequences of sequence SEQ ID NO: 26, a diabody consisting of two acid sequences amines of sequence SEQ ID NO: 28, a diabody consisting of two amino acid sequences of sequence SEQ ID NO: 33 or a diabody consisting of two amino acid sequences of sequence SEQ ID NO: 34,
a single chain diabody,
a minobody such as scFv-CH3, especially a scFv-CH3 consisting of the amino acid sequence SEQ ID NO: 14 or a scFv-CH3 consisting of the amino acid sequence SEQ ID NO: 29, and the diabody-CH3, in particular a diabody-CH3 consisting of two sequences of amino acids of sequence SEQ ID NO: 15 or a diabody-CH3 consisting of two amino acid sequences of sequence SEQ ID NO: 30, the scFv-Fc, in particular a scFv-Fc consisting of the acid sequence amino SEQ ID NO: 16 or a scFv-Fc consisting of the amino acid sequence SEQ ID NO: 31, or
a diabody-Fc.

7. A pharmaceutical composition comprising as active substance a peptide construct containing no CH1 region, recognizing the SAG1 antigen of Toxoplasma gondii and capable of neutralizing the invasion of the cells by Toxoplasma gondii, optionally in combination with a pharmaceutically acceptable vehicle.

8. The pharmaceutical composition according to claim 7, wherein the peptide construct comprises the variable regions of the heavy chain and of the light chain of a first antibody recognizing the SAG1 antigen of Toxoplasma gondii, in particular the monoclonal antibody 4F11E12, and which may contain all or part of the constant region devoid of CH1 region, of the heavy chain of a second antibody, in particular a murine IgG2a immunoglobulin, said peptide construct recognizing the SAG1 antigen of Toxoplasma gondii and being capable of neutralizing the invasion cells by Toxoplasma gondii.

9. The pharmaceutical composition according to claim 7 wherein said peptide construct recognizes the conformational epitope formed by the amino acids at positions 35 to 37, 39, 41, 42, 45, 48, 50, 59 to 65 and 112 to 114 of the amino acid sequence SEQ ID NO: 3.

10. The pharmaceutical composition according to claim 7, wherein said peptide construct comprises the following six CDRs:
a CDR1 having at least 95%, at least 96%, at least 97%, at least 98% or at least 99% % identity with the sequence SEQ ID NO: 4, or consisting of the amino acid sequence SEQ ID NO: 4,
a CDR2 having at least 95%, at least 96%, at least 97%, at least 98% or at least 99% identity with the sequence SEQ ID NO: 5, or consisting of the amino acid sequence SEQ ID NO: 5,
a CDR3 having at least 95%, at least 96%, at least 97% at least 98% or at least 99% identity with the sequence SEQ ID NO: 6, or consisting of the amino acid sequence SEQ ID NO: 6,
a CDR4 having at least 95%, at least 96%, at least 97%, at least 98% or at least 99% identity with the sequence SEQ ID NO: 7, or consisting of the amino acid sequence SEQ ID NO: 7,
a CDR5 having at least 95%, me ns 96%, at least 97%, at least 98% or at least 99% identity with the sequence SEQ ID NO: 8, or consisting of the amino acid sequence SEQ ID NO: 8, and
a CDR6 having at least at least 95%, at least 96%, at least 97%, at least 98% or at least 99% identity with the sequence SEQ ID NO: 9, or consisting of the amino acid sequence SEQ ID NO: 9,
provided that said peptide construct retains its ability to neutralize cell invasion by Toxoplasma gondii.

11. The pharmaceutical composition according to claim 7, wherein said peptide construct is devoid of the CH2 and CH3 regions of said second antibody, or comprises a CH3 region and is devoid of CH2 region of said second antibody, or comprises a CH2 region and a CH3 region of the aforesaid second antibody.

12. The pharmaceutical composition according to claim 7, in which the peptide construct is chosen from:
scFv, in particular a scFv consisting of the amino acid sequence SEQ ID NO: 10, a scFv consisting of the sequence of amino acids SEQ ID NO: 12, a scFv consisting of the amino acid sequence SEQ ID NO: 17, a scFv consisting of the amino acid sequence SEQ ID NO: 20, a scFv consisting of the amino acid sequence SEQ ID NO: 21, a scFv consisting of the amino acid sequence SEQ ID NO: 22, a scFv consisting of the amino acid sequence SEQ ID NO: 23, a scFv consisting of the amino acid sequence SEQ ID NO: 24, a scFv consisting of the amino acid sequence SEQ ID NO: 25, a scFv consisting of the amino acid sequence SEQ ID NO: 27, a scFv consisting of the amino acid sequence SEQ ID NO: 32, a scFv consisting of the amino acid sequence SEQ ID NO: 35, a scFv consisting of the sequence number of amino acids SEQ ID NO: 36, a scFv consisting of the amino acid sequence SEQ ID NO: 37, a scFv consisting of the amino acid sequence SEQ ID NO: 38 or a scFv consisting of the amino acid sequence amino acids SEQ ID NO: 39,
a diabody, in particular a diabody consisting of two amino acid sequences of sequence SEQ ID NO: 11, a diabody consisting of two amino acid sequences of sequence SEQ ID NO: 13, a diabody consisting of two amino acid sequences of sequence SEQ ID NO: 18, a diabody consisting of two amino acid sequences of sequence SEQ ID NO: 19, a diabody consisting of two amino acid sequences of sequence SEQ ID NO: 26, a diabody consisting of two amino acid sequences of sequence SEQ ID NO: 28, a diabody consisting of two amino acid sequences of sequence SEQ ID NO: 33 or a diabody consisting of two amino acid sequences of sequence SEQ ID NO: 34,
a single chain diabody,
a minibody such as scFv-CH3, in particular a scFv-CH3 consisting of the amino acid sequence SEQ ID NO: 14 or a scFv-CH3 consisting of the amino acid sequence SEQ ID NO: 29, and the diabodies —CH3, in particular a diabody-CH3 consisting of two amino acid sequences of sequence SEQ ID NO: 15 or a diabody-CH3 consisting of two amino acid sequences of sequence SEQ ID NO: 30, the scFv-Fc, especially a scFv-Fc consisting of the amino acid sequence SEQ ID NO: 16 or a scFv-Fc consisting of the amino acid sequence SEQ ID NO: 31, or
a diabody-Fc.

13. A peptide construct not containing a CH1 region, recognizing the SAG1 antigen of Toxoplasma gondii and capable of neutralizing the invasion of the cells by Toxoplasma gondii, in particular comprising the variable regions of the heavy chain and the light chain of a first antibody recognizing the SAG1 antigen of Toxoplasma gondii, in particular the monoclonal antibody 4F11E12, and which may contain all or part of the constant region devoid of CH1 region, of the heavy chain of a second antibody, in particular a murine IgG2a immunoglobulin, subject to that said peptide construct is different from the sequence SEQ ID NO: 10.

14. The peptide construct according to claim 13, comprising the following six CDRs:
a CDR1 having at least 95%, at least 96%, at least 97%, at least 98% or at least 99% identity with the sequence SEQ ID NO: 4, or consisting of the amino acid sequence SEQ ID NO: 4,
a CDR2 having at least 95%, at least 96%, at least 97%, at least 98% or at least 99% identity with the sequence SEQ ID NO: 5, or consisting of the amino acid sequence SEQ ID NO: 5,
a CDR3 having at least 95%, at least 96%, at least 97%, at least 98% or at least 99% identity with the sequence SEQ ID NO: 6, or consisting of the amino acid sequence SEQ ID NO: 6,
a CDR4 having at least 95%, at least 96%, at least 97%, at least 98% or at least 99% identity with the sequence SEQ ID NO: 7, or consisting of the amino acid sequence SEQ ID NO: 7,
a CDR5 having at least 95%, at least 96%, at least 97%, minus 98% or at least 99% of ident with the sequence SEQ ID NO: 8, or consisting of the amino acid sequence SEQ ID NO: 8, and
a CDR6 having at least 95%, at least 96%, at least 97%, at least 98% or at least at least 99% identity with the sequence SEQ ID NO: 9, or consisting of the amino acid sequence SEQ ID NO: 9,
provided that said peptide construct retains its ability to neutralize the invasion of the cells by Toxoplasma gondii.

15. The peptide construct according to claim 13, chosen from:
scFv, in particular a scFv consisting of the amino acid sequence SEQ ID NO: 12, a scFv consisting of the amino acid sequence SEQ ID NO: 17, a scFv consisting of the amino acid sequence SEQ ID NO: 20, a scFv consisting of the amino acid sequence SEQ ID NO: 21, a scFv consisting of the amino acid sequence SEQ ID NO: 22, a scFv constituted of the amino acid sequence SEQ ID NO: 23, a scFv consisting of the amino acid sequence SEQ ID NO: 24, a scFv consisting of the amino acid sequence SEQ ID NO: 25, a scFv consisting of amino acid sequence SEQ ID NO: 27, a scFv consisting of the amino acid sequence SEQ ID NO: 32, a scFv consisting of the amino acid sequence SEQ ID NO: 35, a scFv consisting of the amino acid sequence amino acid SEQ ID NO: 36, a scFv consisting of the amino acid sequence SEQ ID NO: 37, a scFv consisting of the amino acid sequence SEQ ID NO: 38 or a scFv consisting of the amino acid sequence SEQ ID NO: 39,
a diabody, in particular a diabody consisting of two amino acid sequences of sequence SEQ ID NO: 11 a diabody consisting of two amino acid sequences of sequence SEQ ID NO: 13, a diabody consisting of two amino acid sequences of sequence SEQ ID NO: 18, a diabody consisting of two amino acid sequences of sequence SEQ ID NO: 19, a diabody consisting of two amino acid sequences of sequence SEQ ID NO: 26, a diabody consisting of two amino acid sequences of sequence SEQ ID NO: 28, a diabody consisting of two acid sequences amines of sequence SEQ ID NO: 33 or a diabody consisting of two amino acid sequences of sequence SEQ ID NO: 34,
a single chain diabody,
a minibody such as scFv-CH3, in particular a scFv-CH3 consisting of the amino acid sequence SEQ ID NO: 14 or a scFv-CH3 consisting of the amino acid sequence SEQ ID NO: 29, and diabody-CH3, in particular a diabody-CH3 consisting of two amino acid sequences of sequence SEQ ID NO: 15 or a diabody-CH3 consisting of two amino acid sequences of sequence SEQ ID NO: 30, scFv-Fc, in particular a scFv-Fc consisting of the amino acid sequence SEQ ID NO: 16 or a scFv-Fc consisting of the acid sequence amino SEQ ID NO: 31, or
a diabody-Fc.
